SEQUENCE LISTING (109 sequences)

SEQ ID NO: 1 - protein, 122 aa, Homo sapiens
EVQLLESGGGLVQPGGSLRLSCAASGFAFSSYGMSWVRQAPGKGLEWVSAFSGSGTSTYYADSVKGRFTISRDKSKNTLYLQMNSLRAEDTAIYYCAKPPGPYIVVAGLFENWGRGTLVTVS

SEQ ID NO: 2 - DNA, 366 bp, Homo sapiens
gaggtgcagctgttggagtctgggggaggcttggtacagcctggggggtccctgagactctcctgtgcagcctctggattcgcctttagcagctatggcatgagctgggtccgccaggctccagggaaggggctggagtgggtctcagcatttagtggaagtggtactagcacatactacgcagactccgtgaagggccggttcaccatctccagagacaaatccaagaacactctgtatctgcaaatgaacagcctgagagccgaagacacggccatttattactgtgcgaagcccccggggccctatatagtagtggctggtctttttgaaaactggggccggggaaccctggtcaccgtctcc

SEQ ID NO: 3 - protein, 110 aa, Homo sapiens
EIVMTQSPATLSVSPGERVTLSCRASQSITDGLAWYQQKSGQAPRLLIYGASTRATGIPARFSGSGSGTEFTLTISSLQSEDFAVYYCQQYNNWPPVTFGQGTKVEIKRT

SEQ ID NO: 4 - DNA, 330 bp, Homo sapiens
gaaattgtgatgacgcagtctccagccaccctgtctgtgtctccaggggaaagagtcaccctctcctgcagggccagtcagagtattaccgacggcttagcctggtaccagcagaaatctggccaggctcccaggctcctcatctatggtgcatccaccagggccactggtatcccagccaggttcagtggcagtgggtctgggacagagttcactctcaccatcagcagcctgcagtctgaagattttgcagtttattactgtcagcagtataataactggcctccggtaacgttcggccaagggaccaaggtggagatcaaacgtacg

SEQ ID NO: 5 - protein, 118 aa, Homo sapiens
EVQLVESGAEVKKPGASVKVSCKASGYTFSRYYIHWVRQAPGQELEWMGWINPDSGDTNYAQKFRGRVTMARDTSISTVYMELNSLRYDDTAVYFCARIIWSSSGMDVWGQGTTVTVS

SEQ ID NO: 6 - DNA, 354 bp, Homo sapiens
gaggtgcagctggtggagtctggggctgaggtgaagaagcctggggcctcagtgaaggtctcctgcaaggcttctggatacaccttcagcagatactatatacactgggtgcgacaggcccctggacaagagcttgagtggatgggatggatcaaccctgacagtggtgacacaaactatgcacagaagtttcggggccgggtcaccatggccagggacacgtccatcagcacagtctacatggagttgaacagcctgagatatgacgacacggccgtgtatttttgtgcaagaatcatttggagttcttccggtatggacgtctggggccaagggaccacggtcaccgtctct

SEQ ID NO: 7 - protein, 216 aa, Homo sapiens
QSALTQPASVSGSPGQSITISCTGTSSDVGSYNLVSWYQQHPGKAPKLMIYEVSKRPSGVSNRFSGSKSGNTASLTISGLQAEDEADYYCHLYVGSSNWVFGGGTKLAVLGQPKAAPSVTLFPPSSKELQANKATLVCPISDFYPGAVTVAWKADSSPVKAGVETTTPSKQSNNKYAASSYLSLTPEQWKSHKSYSCQVTHEGSTVEKTVAPTECS

SEQ ID NO: 8 - DNA, 651 bp, Homo sapiens
cagtctgccctgactcagcctgcctctgtgtctgggtctcctggacagtcgatcaccatctcctgcactggaaccagcagtgatgttgggagttataatcttgtctcctggtatcaacaacacccaggcaaggcccccaaactgatgatttatgaggtcagtaagcggccctcaggggtttctaatcgcttctctggctccaagtctggcaacacggcctccctgacaatctctgggctccaggctgaggacgaggctgattattactgccacttatatgtaggtagtagtaactgggtgttcggcggagggaccaagctggccgtcctaggtcagcccaaggctgccccctcggtcactctgttcccaccctcctctaaggagcttcaagccaacaaggccacactggtgtgtcccataagtgacttctacccgggagccgtgacagtggcctggaaggcagatagcagccccgtcaaggcgggagtggagaccaccacaccctccaaacaaagcaacaacaagtacgcggccagcagctacctgagcctgacgcctgagcagtggaagtcccacaaaagctacagctgccaggtcacgcatgaagggagcaccgtggagaagacagtggcccctacagaatgttcatag

SEQ ID NO: 9 - protein, 122 aa, Homo sapiens
EVQLVESGGGVVQPGRSLRLSCAASGFSFSNYAMHWVRQAPGKGLEWVALISFDGSDKYYADSVKGRFTMSRDNSKNTLYLQMNSLRAEDTAVYYCARDEPYCGGDCFAFDVWGQGTMVTVS

SEQ ID NO: 10 - DNA, 366 bp, Homo sapiens
gaggtgcagctggtggagtctgggggaggcgtggtccagcctgggaggtccctgagactctcctgtgcagcctctggattctccttcagtaactatgctatgcactgggtccgccaggctccaggcaaggggctggagtgggtggcacttatatcatttgatggaagcgataaatactacgcagactccgtgaagggccgattcaccatgtccagagacaattccaagaacaccctgtatctgcaaatgaacagcctgagagctgaggacacggctgtgtattactgtgcgagagatgagccatattgtggtggtgactgcttcgcttttgatgtctggggccaagggacaatggtcaccgtctct

SEQ ID NO: 11 - protein, 111 aa, Homo sapiens
DIQLTQSPSSLSASAGDRVSMTCRASQIISSSLNWYQQKPGKAPKLLIYAASNLQSGVPSRFSGSGSGTDFTLTISSLQPEDFATYYCQQSYSTPPELTFGGGTKVDIKRT

SEQ ID NO: 12 - DNA, 333 bp, Homo sapiens
gacatccagttgacccagtctccatcctccctgtctgcatctgccggagacagagtcagcatgacttgccgggcaagtcagatcattagtagttccttaaattggtatcagcagaaaccagggaaagcccctaaactcctgatctatgctgcatccaatttacaaagtggggtcccatcaaggttcagtggcagtggatctgggacagatttcactctcaccatcagcagtctgcaacctgaagattttgcaacttactactgtcaacagagttacagtacccctccggagctcactttcggcggagggaccaaagtggatatcaaacgtacg

SEQ ID NO: 13 - protein, 120 aa, Homo sapiens
QVQLQESGPGLVKPSETLSLTCSVSGGSINYYYWSWIRQPPGKGLECIGYISDTGRTYYNPSLRSRVTISVDTSKNQFSLNLTSMTAADTAVYYCARDPTGSRQKNAFDMWGQGTMVTVS

SEQ ID NO: 14 - DNA, 360 bp, Homo sapiens
caggtgcagctgcaggagtcgggcccaggactggtgaagccttcggagaccttgtccctcacctgctctgtctctggtggctccatcaattattattactggagctggatccggcagccccccgggaagggactggagtgcattggctatatctctgacactgggagaacctactacaacccctccctcaggagtcgagtcaccatatcagtagacacctccaagaaccagttctcccttaacctgacctctatgaccgctgcggacacagccgtgtattactgtgcgagagaccctactgggagccgccaaaaaaatgcttttgatatgtggggccaagggacaatggtcaccgtgtct

SEQ ID NO: 15 - protein, 109 aa, Homo sapiens
AIQMTQSPSSLSASVGDRVTITCRASQNIRSYLNWYQQKPGKAPNLLIYGTTSLESGVPSRFSGSGSGTDFTLTISSLQPEDFATYYCQQSYHTPQTFGQGTKVEIKRT

SEQ ID NO: 16 - DNA, 327 bp, Homo sapiens
gccatccagatgacccagtctccatcctccctgtctgcatctgtaggagacagagtcaccatcacttgccgggcaagtcagaatattaggagttacttaaattggtatcagcagaagccagggaaagcccctaacctcctgatttatggtaccaccagtttggagagtggggtcccatcacggttcagtggcagtggatctgggacagatttcactctcaccatcagcagtctgcaacctgaagattttgcgacttactactgtcaacagagttaccatacccctcaaacgttcggccaagggaccaaggtggagatcaaacgtacg

SEQ ID NO: 17 - protein, 118 aa, Homo sapiens
EVQLLESGGGVVQPGRSLRLSCAASGFTFRTYGMHWVRQAPGKGLEWVAVISHDGNLKYYGGSVKGRFTISRDNSKNALYLQMNSLRVDDTALYYCARGIGITDAFDIRGQGTMVVVS

SEQ ID NO: 18 - DNA, 354 bp, Homo sapiens
gaggtgcagctgttggagtcggggggaggcgtggtccagcctgggaggtccctgagactctcctgtgcagcctctggcttcacctttaggacctatggcatgcactgggtccgccaggctcctggcaaggggctggagtgggtggcagtcatctcacatgatggaaatttaaaatattatggaggctctgtgaagggccgattcaccatctccagagacaattccaagaacgcgctgtatttgcaaatgaacagcctgagagttgacgacacggctctgtattactgtgcgagaggtatcggtataaccgatgcttttgatatccggggccaagggacaatggtcgtcgtctct

SEQ ID NO: 19 - protein, 235 aa, Homo sapiens
MAWMMLLLTLLIHCTGSWAQSVLTQPPSVSAAPGQKVTISCSGSSSNIGKNYVSWYQQLPGTAPKLLIYDNDKRPSGIPDRFSGSKSGTSATLGITGLQTGDEADYHCGAWDSSLNGGVFGGGTKVTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 20 - DNA, 708 bp, Homo sapiens
atggcctggatgatgcttctcctcacccttctcattcactgcacagggtcctgggcccagtctgtgttgacgcagccgccctcagtgtctgcggccccaggacagaaggtcaccatctcctgttctggaagcagctccaacattgggaaaaattatgtatcctggtaccagcaactcccaggaacagcccccaaactcctcatttatgacaatgataagcgaccctcagggattcctgaccgattctctggctccaagtctggcacgtcagccaccctgggcatcaccggactccagactggggacgaggccgattatcactgcggagcatgggatagcagtctgaatgggggggtgttcggcggagggaccaaggtgaccgtcctaggtcagcccaaggctgccccctcggtcactctgttcccgccctcctctgaggagcttcaagccaacaaggccacactggtgtgtctcataagtgacttctacccgggagccgtgacagtggcctggaaggcagatagcagccccgtcaaggcgggagtggagaccaccacaccctccaaacaaagcaacaacaagtacgcggccagcagctacctgagcctgacgcctgagcagtggaagtcccacagaagctacagctgccaggtcacgcatgaagggagcaccgtggagaagacagtggcccctacagaatgttcatag

SEQ ID NO: 21 - protein, 123 aa, Homo sapiens
EVQLVESGAEVKKPGSSVKVSCKASGGTFRSYAVSWVRQAPGQGLEWMGGIIAIFGTTNYAQKFQDRVTITADDLPSTAYMELTSLRSEDTAVYFCVRGRGYYLGGDYGSLDSWGQGTLVTVS

SEQ ID NO: 22 - DNA, 369 bp, Homo sapiens
gaggtgcagctggtggagtctggggctgaggtgaagaagcctgggtcctcggtgaaggtctcctgcaaggcctctggaggcaccttcagaagctatgctgtcagctgggtgcgacaggcccctggacaagggcttgagtggatgggagggatcattgctatctttggaacaacaaactacgcacagaagttccaggacagagtcacgattaccgcggacgatctcccgagcacagcctacatggagttgaccagcctgagatctgaggacacggccgtttatttctgtgtgagagggcgtggatattacctgggcggtgactacgggtcccttgactcctggggccagggaaccctggtcaccgtctcc

SEQ ID NO: 23 - protein, 109 aa, Homo sapiens
DIQMTQSPSSLSASVGDRITITCRASQGIDNYLAWFQQKPGKVPELLIYAASTLQSGVPSRFSGSGSGTGFTLTISSLQPEDVATYYCQKYDSAPLTFGGGTKVDIKRT

SEQ ID NO: 24 - DNA, 327 bp, Homo sapiens
gacatccagatgacccagtctccatcctccctgtctgcatctgtaggagacagaatcaccatcacttgccgggcgagtcagggcattgacaattatttagcctggtttcagcagaaaccagggaaagttcctgagctcctgatttatgctgcatccactctgcaatcaggggtcccatctcggttcagcggcagtggatctgggacgggattcactctcaccatcagcagcctgcagcctgaagatgttgcaacttattactgtcaaaagtatgacagtgccccgctcactttcggcggagggaccaaagtggatatcaaacgtacg

SEQ ID NO: 25 - protein, 119 aa, Homo sapiens
EVQLVESGAGVKKPGSSVKVSCKASGNTFSSNAISWVRQAPGQGLEWMGGILGMLRTTNYAQKFQGRVTITADEFMNTAYMELSSLTSEDTAVYYCARSSGYYPDYFHLWGQGTLVTVS

SEQ ID NO: 26 - DNA, 366 bp, Homo sapiens; identical to SEQ ID NO: 2

SEQ ID NO: 27 - protein, 110 aa, Homo sapiens; identical to SEQ ID NO: 3

SEQ ID NO: 28 - DNA, 330 bp, Homo sapiens; identical to SEQ ID NO: 4

SEQ ID NO: 29 - protein, 123 aa, Homo sapiens
EVQLVESGGGLVQPGRSLRLSCVGSGFNFDGYAMQWVRQVPGKGLEWVSGISWHSDSTGYADSVRGRFTISRDNAKNSLYLQMDSLRPEDTAFYYCGKSIGHVGGSYRHPIDYWGQGTLVTVS

SEQ ID NO: 30 - DNA, 369 bp, Homo sapiens
gaggtgcagctggtggagtctgggggaggcttagtccagcctggcaggtccctgagactctcctgtgtagggtctggattcaactttgatggatatgccatgcagtgggtccggcaagttccagggaagggcctggagtgggtctcaggtattagttggcacagtgatagtactggctatgcggactctgtgaggggccgattcaccatctccagagacaacgccaagaactccctgtatttgcaaatggacagtctgagacctgaggacacggccttctattactgtggaaaatccatcggccatgttggaggcagttatcgacatcccattgactactggggccagggaaccctggtcaccgtctcc

SEQ ID NO: 31 - protein, 109 aa, Homo sapiens
DIQLTQSPSSLSASVGDRVTITCRASQGITDYLAWYQQKPGTIPKLLIYAASTLQSGVPSRFSGGGYGTDFTLTISSLQPEDVATYYCQKYNSAPLTFGGGTKVEIKRT

SEQ ID NO: 32 - DNA, 327 bp, Homo sapiens
gacatccagttgacccagtctccatcctccctgtctgcatctgtaggagacagagtcaccatcacttgccgggcgagtcagggcattaccgattatttagcctggtatcagcagaaaccagggacaattcctaagctcctgatctatgctgcatccactttgcaatcaggggtcccatctcggttcagtggcggtggatatgggacagatttcactctcaccatcagcagcctgcagcctgaagatgttgcaacttattactgtcaaaagtataacagtgccccgctcactttcggcggagggaccaaggtggaaatcaaacgtacg

SEQ ID NO: 33 - protein, 126 aa, Homo sapiens
EVQLVESGAEVKKPGASVKVSCKASGYTFTSFDINWVRQAPGQGLEWMGWMTPTSGKTGYAQKFQGRITMTRNTSINTAYMELSSLRSEDTAVYFCARGPERRTTIFDVILDWFDPWGQGTLVTVS

SEQ ID NO: 34 - DNA, 378 bp, Homo sapiens
gaggtgcagctggtggagtctggggctgaagtgaagaagcctggggcctcagtgaaggtctcctgtaaggcttctggatacaccttcaccagttttgatattaactgggtgcgacaggcccctggacaaggacttgagtggatgggatggatgaccccaaccagtggaaagacaggctacgcacagaagttccagggcagaatcaccatgaccaggaacacctccataaatacagcctacatggagttgagcagcctgagatctgaggacacggccgtatatttctgtgcgagaggccccgagagacgtactacaatttttgacgtgattctggactggttcgatccctggggccagggaaccctggtcaccgtctcc

SEQ ID NO: 35 - protein, 109 aa, Homo sapiens
DIVMTQSPFSLSLSPGERATLSCRASQSISNYLAWYQQKPGQAPRLLIYDSSNRATGVPARFSGSGSGTDFTLTISSLEPEDFAVYYCQQRSNWMYTFGQGTKVEIKRT

SEQ ID NO: 36 - DNA, 327 bp, Homo sapiens
gacatcgtgatgacccagtctccattctccctgtctttgtctccaggggaaagagccaccctctcctgcagggccagtcagagtattagcaactacttagcctggtaccaacagaaacctggccaggctcccaggctcctcatctatgattcatccaacagggccactggcgtcccagccaggttcagtggcagtgggtctgggacagacttcactctcaccatcagcagcctagagcctgaagattttgcagtttattactgtcagcagcgtagcaactggatgtacacttttggccaggggaccaaggtggaaatcaaacgtacg

SEQ ID NO: 37 - protein, 118 aa, Homo sapiens
  misc_feature (31)..(31): Xaa can be any naturally occurring amino acid
QVQLQESGPGLVRPSETLSLTCNVSGDSINXYYWSWIRQPPGKGLEWIGYIYYSGSTNYNPSLNSRVTISVEMSKNQFSLILNSVTAADTAVYYCARVDYDSGGSFDPWGQGTLVTVS

SEQ ID NO: 38 - DNA, 354 bp, Homo sapiens
caggtgcagctgcaggagtcgggcccagggctggtgaggccttcggagaccctgtccctcacctgcaatgtctctggtgactccatcaatgattactactggagctggatccggcagcccccagggaagggactggagtggattgggtacatctattacagtgggagcaccaactacaacccctccctcaacagtcgagtcaccatttcggtagaaatgtccaagaaccagttctccctgattctgaactctgtgaccgctgcggacacggccgtgtattactgtgccagagtggactatgatagtggtgggtctttcgacccctggggccagggaaccctggtcaccgtctcc

SEQ ID NO: 39 - protein, 236 aa, Homo sapiens
MAWMMLLLTLLAHCTGSWAQSVLTQPPSVSGAPGQRVTISCTGSSSNIGAGYDVHWYQQLPGTAPKLLIYGNSNRPSGVPDRFSGSKSGTSASLAITGLQAEDEADFYCQSYDSRLSGYVFGTGTKVTVLGQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTECS

SEQ ID NO: 40 - DNA, 711 bp, Homo sapiens
atggcctggatgatgcttctcctcactctcctcgctcactgcacagggtcctgggcccagtctgtgctgacgcagccgccctcagtgtctggggccccagggcagagggtcaccatctcctgcactgggagcagctccaacatcggggcaggatatgatgttcactggtaccagcaacttccaggaacagcccccaaactcctcatctatggtaacagcaatcggccctcaggggtccctgaccgattctctggctccaagtctggcacctcagcctccctggccatcactgggctccaggctgaggatgaggctgatttttactgccagtcctatgacagcagactgagtggttatgtcttcggaactgggaccaaggtcaccgtcctaggtcagcccaaggccaaccccactgtcactctgttcccgccctcctctgaggagctccaagccaacaaggccacactagtgtgtctgatcagtgacttctacccgggagctgtgacagtggcctggaaggcagatggcagccccgtcaaggcgggagtggagaccaccaaaccctccaaacagagcaacaacaagtacgcggccagcagctacctgagcctgacgcccgagcagtggaagtcccacagaagctacagctgccaggtcacgcatgaagggagcaccgtggagaagacagtggcccctacagaatgttcatag

SEQ ID NO: 41 - protein, 122 aa, Homo sapiens; identical to SEQ ID NO: 1

SEQ ID NO: 42 - DNA, 366 bp, Homo sapiens
gaggtgcagctgttggagtctgggggaggcttggtacagcctggggggtccctgagactctcctgtgcagcctctggattcgcctttagcagctatggcatgagctgggtccgccaggctccagggaaggggctggagtgggtctcagcatttagtggaagtggtactagcacatactacgcagactccgtgaagggccggttcaccatctccagagataaatccaagaacactctgtatctgcaaatgaacagcctgagagccgaagacacggccatttattactgtgcgaagcccccggggccctatatagtagtggctggtctttttgaaaactggggccggggaaccctggtcaccgtctcc

SEQ ID NO: 43 - protein, 236 aa, Homo sapiens
MAWALLLLTLLAHCTGSWAQSVLTQPPSVSGAPGQRVTISCTGTSSNIGAGYEVHWYQQLPGTAPKLIINANTNRPSGVPDRFSGSQSGTSASLAITGLRAEDEADYYCQSFDSSLSGWLFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 44 - DNA, 711 bp, Homo sapiens
atggcctgggctcttctcctcctcactctcctcgctcactgcacagggtcctgggcccagtctgtgctgacgcagccgccctcagtgtctggggccccagggcagagggtcaccatctcctgcactgggaccagctccaacatcggggccggctatgaagtacactggtaccagcaacttccaggaacagcccccaaactcatcatcaatgctaataccaatcggccctcaggggtccctgaccgattctctggctcccagtctggcacctcagcctccctggccatcactggtctccgggctgaggatgaggctgattattactgccagtcctttgacagcagcctgagtggttggctgttcggcggagggaccaaactgaccgtcctaggtcagcccaaggctgccccctcggtcactctgttcccgccctcctctgaggagcttcaagccaacaaggccacactggtgtgtctcataagtgacttctacccgggagccgtgacagtggcctggaaggcagatagcagccccgtcaaggcgggagtggagaccaccacaccctccaaacaaagcaacaacaagtacgcggccagcagctatctgagcctgacgcctgagcagtggaagtcccacagaagctacagctgccaggtcacgcatgaagggagcaccgtggagaagacagtggcccctacagaatgttcatag

SEQ ID NO: 45 - protein, 128 aa, Homo sapiens
  misc_feature (9)..(9): Xaa can be any naturally occurring amino acid
VQLLESGGXVVQPGRSLRLSCAASGFMFSSYVMHWVRQPPGKGLEWVAVIWYDGSKTYFADSMRGRLTVSRDNSKNALYLQMNRLRAEDTAVYYCARQQDSGYSGPEVSYYSHYGMDVWGQGTTVIVS

SEQ ID NO: 46 - DNA, 387 bp, Homo sapiens
  misc_feature (30)..(30): n is a, c, g, or t
gaggtgcagctgttggagtctgggggaggngtggtccagcctgggaggtccctgagactctcctgtgcagcgtctggattcatgttcagcagttatgtcatgcactgggtccgccagcctccaggcaaggggctggagtgggtggctgttatatggtatgatggaagtaagacttatttcgcagactccatgaggggccgactcaccgtctccagagacaattccaagaacgcgctgtatctccaaatgaacagactgagagccgaggacacggctgtgtattactgtgcgagacagcaggatagtggatatagtggcccagaagtctcttattattctcattatggtatggacgtctggggccaagggaccacggtcatcgtctcc

SEQ ID NO: 47 - protein, 237 aa, Homo sapiens
MAWAPLLLTLLTHCAGSWAQSVLTQSPSASGTPGRAITISCSGSSSNIGSNPVNWYQQLPGAAPKLLIYADEHRPSGVPDRFSGPKSGTSASLAISGLQSEDEADYYCAAWDDSLSGPAVVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 48 - DNA, 731 bp, Homo sapiens
aagcttgccaccatggcctgggctccgctcctcctcaccctcctcactcactgtgcagggtcctgggcccagtctgtgctgactcagtcaccctcagcgtctgggacccccgggcgggcaatcaccatctcttgttctggaagcagctccaacatcggaagtaatcctgtaaattggtatcagcaactcccaggagcggcccccaaactcctcatctatgctgatgaacaccggccctcaggggtccctgaccgattctctggccccaagtctggcacctcagcctccctggccatcagtgggctccagtctgaggatgaggctgattattattgtgcagcatgggatgacagcctgagtggtccggctgtggttttcggcggggggaccaagctgaccgtcctaggtcagcccaaggctgccccctcggtcactctgttcccgccctcctctgaggagcttcaagccaacaaggccacactggtgtgtctcataagtgacttctacccgggagccgtgacagtggcctggaaggcagatagcagccccgtcaaggcgggagtggagaccaccacaccctccaaacaaagcaacaacaagtacgcggccagcagctatctgagcctgacgcctgagcagtggaagtcccacagaagctacagctgccaggtcacgcatgaagggagcaccgtggagaagacagtggcccctacagaatgttcatagaattc

SEQ ID NO: 49 - protein, 121 aa, Homo sapiens
QVQLKESGPGLVKPSETLSLTCTVSGGSISSSNYNWGWIRQPPGKGLEWIGSVYYSGSTYFNPSLKSRVTISVDTSKNQFSLKLTSVTAADTAVYYCARLIAFYYDTNGYTWGQGTLVTVS

SEQ ID NO: 50 - DNA, 363 bp, Homo sapiens
caggtgcagttgaaggagtcgggcccaggactggtgaagccttcggagaccctgtccctcacctgcactgtctctggtggctccatcagcagtagtaactacaactggggctggatccgccagcccccagggaaggggctggagtggattgggagtgtctattatagtgggagcacctacttcaacccgtccctcaagagtcgagtcaccatatccgtagacacgtccaagaaccagttctccctgaagctgacctctgtgaccgccgcggacacggctgtgtattactgtgccagactaatagcattttactatgataccaatggttatacttggggccagggaaccctggtcaccgtctcc

SEQ ID NO: 51 - protein, 109 aa, Homo sapiens
NSIMTQSPFTLSASVGDKVTITCRASQSISPWLAWYQQKPGKAPKLLIYKASSLETGVPSRFSGSESGTEFTLTISGLQPDDFATYYCQQYSSFARTFGQGTKVEIKRT

SEQ ID NO: 52 - DNA, 327 bp, Homo sapiens
aattcgattatgacccagtctccattcaccctgtctgcgtctgtaggagacaaagtcaccatcacttgccgggccagtcagagtattagtccctggttggcctggtatcagcagaaaccagggaaagcccctaaactcctgatctataaggcatctagtttagaaactggggtcccatcaagattcagcggcagtgaatctgggacagaattcactctcaccatcagcggcctgcagcctgatgattttgcaacttattactgccaacagtattctagttttgcaaggacgttcggccaagggaccaaggtggaaatcaaacgaact

SEQ ID NO: 53 - protein, 123 aa, Homo sapiens
QVQLQESAPGLVKPSGTLSLTCAVSGASLSSSDWWTWVRQPPGKGLEWIGEIHHGGSTNSNPSLQSRVTISIDKSKKQFSLKLTSVTVADTAVYYCARAPGADAGPYSENIHHWGQGTLVTVS

SEQ ID NO: 54 - DNA, 369 bp, Homo sapiens
caggtgcagctgcaggagtcggccccgggcctggtgaagccttcggggaccctgtccctcacctgcgctgtctctggtgcctccctcagcagttctgactggtggacttgggtccgccagcccccaggcaagggcctggagtggattggggaaatccatcatggaggcagtaccaactccaacccgtccctccagagtcgagtcaccatatccatagacaagtccaagaagcagttctccctgaagttgacctctgtgaccgtcgcagacacggccgtctattactgtgcgcgagcccccggcgccgatgctggtccttactcggaaaacatccaccactggggccagggcaccctggtcaccgtctcc

SEQ ID NO: 55 - protein, 236 aa, Homo sapiens
MAWALLLLTLLLHCTGSLSQLVLTQSPSASASLGASVKLTCTLSSRHSDYAIGWHQQQPGKGPRYLMKLNPDGSHTKGDGIPDRFSGSSSGAERYLTISNLLSEDEADYYCQTWGTGIQVFGGGTRLTVISQPKAAPSVTLFPPSSEELQANKATLVCLISDFYPGAVTVAWKVDSSPVKAGVETTTPSKQSNNKYAASSYLSLTPEQWKSHKSYSCQVTHEGSTVEKTVAPTECS

SEQ ID NO: 56 - DNA, 728 bp, Homo sapiens
aagcttgccaccatggcctgggctctgctgctcctcaccctcctcctccactgcacagggtctctctcccagcttgtgctgactcaatcgccctctgcctctgcctccctgggagcctcggtcaagctcacctgcactctgagcagtagacacagcgactacgccatcggatggcatcagcagcagccagggaagggccctcgttacttgatgaagcttaaccctgatggcagccacaccaagggggacgggatccctgatcgcttctcaggctccagttctggggctgagcgctacctcaccatctccaacctcctgtctgaggatgaggctgactattactgtcagacctggggcactggcattcaggtgttcggcggagggacccggctgaccgtcataagtcagcccaaggctgccccctcggtcactctgttcccgccctcctctgaggagcttcaagccaacaaggccacactggtgtgtctcataagtgacttctacccgggagccgtgacagtggcctggaaggtagatagcagccccgtcaaggcgggagtggagaccaccacaccctccaaacaaagcaacaacaagtacgcggccagcagctacctgagcctgacgcctgagcagtggaagtcccacaaaagctacagctgccaggtcacgcatgaagggagcaccgtggagaagacagtggcccctacagaatgttcatagaattc

SEQ ID NO: 57 - protein, 122 aa, Homo sapiens; identical to SEQ ID NO: 9

SEQ ID NO: 58 - DNA, 366 bp, Homo sapiens
gaagtgcagctggtggagtctgggggaggcgtggtccagcctgggaggtccctgagactctcctgtgcagcctctggattctccttcagtaactatgctatgcactgggtccgccaggctccaggcaaggggctggagtgggtggcacttatatcatttgatggaagcgataaatactacgcagactccgtgaagggccgattcaccatgtccagagacaattccaagaacaccctgtatctgcaaatgaacagcctgagagctgaggacacggctgtgtattactgtgcgagagatgagccatattgtggtggtgactgcttcgcttttgatgtctggggccaagggacaatggtcaccgtctct

SEQ ID NO: 59 - protein, 236 aa, Homo sapiens
MAWALLLLTLLAHCTGSWAQSVLTQPPSVSGAPGQRVTISCTGSSSNIGAGYDVHWYQQLPGTAPKLLIYANNNRPSGVPDRFSGSKSGTSASLAITGLQAEDEADYYCQSYDSSLSGGVFGTGTTVTVLGQPKANPTVTLFPPSSEELQANKATLVCLISDFYPGAVTVAWKADGSPVKAGVETTKPSKQSNNKYAASSYLSLTPEQWKSHGSYSCQVTHEGSTVEKTVAPTECS

SEQ ID NO: 60 - DNA, 728 bp, Homo sapiens
aagcttgccaccatggcctgggctctgctgcttctcactctcctcgctcactgcacagggtcctgggcccagtctgtgctgacgcagccgccctcagtgtctggggccccagggcagagggtcaccatctcctgcactgggagcagctccaacatcggggcaggttatgatgtacactggtaccagcagcttccaggaacagcccccaaactcctcatctatgctaacaacaatcggccctcaggggtccctgaccgattctctggctccaagtctggcacctcagcctccctggccatcactgggctccaggctgaggatgaggctgattattactgccagtcctatgacagcagcctgagtggaggggtcttcggaactgggaccacggtcaccgtcctaggtcagcccaaggccaaccccactgtcactctgttcccgccctcctctgaggagctccaagccaacaaggccacactagtgtgtctgatcagtgacttctacccgggagctgtgacagtggcctggaaggcagatggcagccccgtcaaggcgggagtggagaccaccaaaccctccaaacagagcaacaacaagtacgcggccagcagctacctgagcctgacgcccgagcagtggaagtcccacggaagctacagctgccaggtcacgcatgaagggagcaccgtggagaagacagtggcccctacagaatgttcatagaattc

SEQ ID NO: 61 - protein, 125 aa, Artificial Sequence
  Synthetic peptide
METDTLLLWVLLLWVPGSWARMENLNKKVDDGFLDIWTYNAELLVLLENERTLDFHDSNVKNLYEKVKSQLKNNASGRLVPRGSPGSGYIPEAPRDGQAYVRKDGEWVLLSTFLGSAWSHPQFEK

SEQ ID NO: 62 - protein, 125 aa, Artificial Sequence
  Synthetic peptide
METDTLLLWVLLLWVPGSWARIQDLEKYVEDTKIDLWSYNAELLVALENQHTIDLTDSEMNKLFEKTRRQLRENASGRLVPRGSPGSGYIPEAPRDGQAYVRKDGEWVLLSTFLGSAWSHPQFEK

SEQ ID NO: 63 - protein, 145 aa, Artificial Sequence
  Synthetic peptide
METDTLLLWVLLLWVPGSWARMENLNKKVDDGFLDIWTYNAELLVLLENERTLDFHDSNVKNLYEKVKSQLKNMKQIEDKIEEILSKIYHIENEIARIKKLIGEAGSAWSHPQFEKGGGSGGGSGGSAWSHPQFEKGGGSGGGSC

SEQ ID NO: 64 - protein, 145 aa, Artificial Sequence
  Synthetic peptide
METDTLLLWVLLLWVPGSWARIQDLEKYVEDTKIDLWSYNAELLVALENQHTIDLTDSEMNKLFEKTRRQLREMKQIEDKIEEILSKIYHIENEIARIKKLIGEAGSAWSHPQFEKGGGSGGGSGGSAWSHPQFEKGGGSGGGSC

SEQ ID NO: 65 - protein, 122 aa, Homo sapiens
EVQLVESGADMKPPGSSVKVPCKASGDTFSSYTITWVRQAPGQGLEWMGGITPIFGSPNYAQRFQDRVIITADESTSTAYMEVSNLRSEDTAVYFCARVGGEWGSGRYYLDHWGQGTLVTVS

SEQ ID NO: 66 - DNA, 366 bp, Homo sapiens
gaggtgcagctggtggagtctggggctgacatgaagccgcctgggtcctcggtgaaagtcccctgcaaggcttctggagacaccttctccagctacactatcacctgggtgcgacaggcccctggacaagggctcgagtggatgggagggatcacccccatctttggttcaccaaactacgcacagaggttccaggacagagtcatcattaccgcggacgaatccactagcacagcctacatggaagtgagcaacctcagatctgaggacacggccgtctatttctgtgcgagggttggaggtgaatggggcagtggccgttactaccttgaccactggggccagggaaccctggtcaccgtctcc

SEQ ID NO: 67 - protein, 110 aa, Homo sapiens
DIQLTQSPASLSVSPGERATLSCRASQSVAGNLAWYQQKPGQAPRLLIYGASTRATGIPARFSGSGSGTEFTLTITSLQSEDFAVYYCQQYNNWPPWTFGQGTKVDIKRT

SEQ ID NO: 68 - DNA, 330 bp, Homo sapiens
gacatccagttgacccagtctccagcctccctgtctgtgtctccaggggaaagagccaccctctcctgcagggccagtcagagtgttgccggcaacttagcctggtaccagcagaaacctggccaggctcccaggctcctcatctatggtgcatccaccagggccactggtatcccagccaggttcagtggcagtgggtctgggacagagttcactctcaccatcaccagcctgcagtctgaagattttgcagtttattactgtcagcagtataataattggcctccgtggacgttcggccaagggaccaaagtggatatcaaacgtacg

SEQ ID NO: 69 - protein, 119 aa, Homo sapiens
EVQLVESGAEVKKPGSSVKVSCKASGGTFITHVFTWVRQAPGQGLEWVGGFIAIFGTSNYAQKFQGRVTITADESTSTAYMELTSLTSEDTAVYYCARGISGSYGWFDPWGQGTLVTVS

SEQ ID NO: 70 - DNA, 357 bp, Homo sapiens
gaggtgcagctggtggagtctggggctgaggtgaagaagcctgggtcctcggtgaaggtctcctgtaaggcttctggaggcaccttcattacccatgtttttacttgggtgcgacaggcccctggacaagggcttgagtgggttggagggttcatcgctatctttggcacatcaaactacgcacagaagttccagggcagagtcacgattaccgcagacgagtctacgagcacagcctacatggagctgaccagcctgacatctgaggacacggccgtgtattactgtgcgagaggaattagtgggagctatgggtggtttgacccctggggccagggaaccctggtcaccgtctcc

SEQ ID NO: 71 - protein, 109 aa, Homo sapiens
DIQLTQSPSSLSASVGDRVTITCQASQDIRKYLNWYQQKPGKAPNLLIYDASNVKTGVPSRFRGSGSGTDFTFTISSLQPEDIATYYCQQYDNLPITFGQGTRLEIKRT

SEQ ID NO: 72 - DNA, 327 bp, Homo sapiens
gacatccagttgacccagtctccatcctccctgtctgcatctgtaggagacagagtcaccatcacttgccaggcgagtcaggacattagaaagtatttgaattggtatcagcagaaaccagggaaagcccctaacctcctgatctacgatgcatccaatgtgaagacaggggtcccttcaaggttccgtggaagtggatctgggacagattttactttcaccatcagcagcctgcagcctgaagatattgcaacatattactgtcaacagtatgataatctcccgatcaccttcggccaagggacacgactggagattaaacgtacg

SEQ ID NO: 73 - protein, 117 aa, Homo sapiens
EVQLQESGPGLVKPSETLSLSCTVSGGSITRSSYYWGWIRQPPGKGLEWIGNIYYSGTTYYNPSLKSRVTISVDTSKNQFSLKLSSVTAADTAVYYCARLYGSGLDYWGQGTLVTVS

SEQ ID NO: 74 - DNA, 351 bp, Homo sapiens
gaggtgcagctgcaggagtcgggcccaggactggtgaagccttcggagaccctgtccctcagttgcactgtctctggtggctccatcactagaagtagttactattggggctggatccgccagcccccagggaaggggctggaatggattgggaatatctattatagtgggaccacgtactacaatccgtccctcaagagtcgagtcaccatatccgtggacacgtctaagaaccagttctccctgaagttgagttctgtgaccgccgcagacacggctgtatattactgtgcgagactctatgggtcggggttagactattggggccagggaaccctggtcaccgtctcc

SEQ ID NO: 75 - protein, 109 aa, Homo sapiens
DIQLTQSPSFLSASVGDRVTITCRASQGSSRYVAWYQQKPGKAPKLLILAASTLHTGVPSRFSGSGSGTEFTLTISSLQPEDFATYSCQQLNGFLLTFGGGTKVEIKRT

SEQ ID NO: 76 - DNA, 327 bp, Homo sapiens
gacatccagttgacccagtctccatccttcctgtctgcatctgtaggagacagagtcaccatcacttgccgggccagtcagggcagtagtcgttatgtagcctggtatcagcaaaagccagggaaagcccctaaactcctgatcttggctgcttccactttgcatactggggtcccatcaaggttcagcggcagtggatctgggacagaattcactctcacaatcagcagcctgcagcctgaagattttgcaacttattcctgtcaacagcttaatggtttcctcctcactttcggcggagggaccaaggtggaaatcaaacgtacg

SEQ ID NO: 77 - protein, 123 aa, Homo sapiens
EVQLVESGPGLVKPSDILSLTCAVSGYSISSNYYWGWIRQPPGKGLEWIGSIYHSGSTYYKPSLESRLGISVDTSKNQFSLKLSFVSAADTAVYYCARHVRSGYPDTAYYFDKWGQGTLVTVS

SEQ ID NO: 78 - DNA, 369 bp, Homo sapiens
gaggtgcagctggtggagtctggcccaggactggtgaagccttcggacatcctgtccctcacctgcgctgtctctggttactccatcagcagtaattactactggggctggattcggcagcccccagggaaggggctggagtggattgggagtatctatcatagtgggagcacctactacaagccgtctctcgagagtcgactcggcatatcagtagacacgtccaagaaccaattctccctgaagttgagttttgtgtccgccgcagacacggccgtgtactattgtgcgcgacatgttaggagtggttaccccgatactgcatactactttgacaagtggggccagggaaccctggtcaccgtctcc

SEQ ID NO: 79 - protein, 233 aa, Homo sapiens [sequence omitted]

SEQ ID NO: 80 - DNA, 702 bp, Homo sapiens
atggcctgggctctgctgcttctcggcctcctctctcactgcacaggctctgtgacctcctatgtgctgactcagccaccctcggtgtcagtggccccaggagagacggccaggatttcctgtgggggaaacaacattggaactaaagttctacactggtaccagcagacgccaggccaggcccctgtgctcgtcgtctatgatgacagcgaccggccctcagggatccctgagcgattctctggctccaactctgggaacacggccaccctgaccatcagcagggtcgaagtcggggatgaggccgactattactgtcaggtgtgggatatcagtactgatcaagcggtattcggcggagggaccaaactgaccgtcctaggtcagcccaaggctgccccctcggtcactctgttcccgccctcctctgaggagcttcaagccaacaaggccacactggtgtgtctcataagtgacttctacccgggagccgtgacagtggcctggaaggcagatagcagccccgtcaaggcgggagtggagaccaccacaccctccaaacaaagcaacaacaagtacgcggccagcagctatctgagcctgacgcctgagcagtggaagtcccacagaagctacagctgccaggtcacgcatgaagggagcaccgtggagaagacagtggcccctacagaatgttcatag

SEQ ID NO: 81 - protein, 122 aa, Homo sapiens
EVQLVQSGAEWKKPGSSVRVSCKASGPIFSSRSISWVRQAPGQGLEWMGGVIPIFGGPTVPQKFQGRLTMTADDSTSTAYMELNRLTSDDTAVYYCARTGYYSGSGTYAFDYWGQGTLVTVS

SEQ ID NO: 82 - DNA, 366 bp, Homo sapiens
gaggtgcagctggtgcagtctggggctgagtggaagaagcctggatcctcggtgagggtctcctgtaaggcctctggacccatcttcagcagcagatcaataagttgggtgcggcaggccccaggacaagggcttgagtggatggggggcgtcatccctatttttggtgggccgaccgtcccccagaagtttcagggcagactcacaatgaccgcggacgactccaccagcacagcctacatggagttgaatcgcctgacatctgacgacacggccgtctattactgtgcgaggacgggatattattctgggtcggggacttatgcctttgactattggggccaaggaaccctggtcaccgtctcc

SEQ ID NO: 83 - protein, 110 aa, Homo sapiens
EIVMTQSPATVSVSPGERATLSCRASQSVSNNLAWYQQKPGQAPRLLIYGASTRATGIPARFSGSGSGTEFTLTISSLQSEDFAIYYCQQYHNWPPLTFGGGTKVEIKRT

SEQ ID NO: 84 - DNA, 330 bp, Homo sapiens
gaaatagtgatgacacagtctccagccaccgtgtctgtgtctccaggggaaagagccaccctctcctgcagggccagtcagagtgttagcaacaacttagcctggtaccagcagaagcctggccaggctcccaggctcctcatctatggtgcatccaccagggccactggtatcccagccaggttcagtggcagtgggtctgggacagagttcactctcaccatcagcagcctgcagtctgaagattttgcaatttattactgtcagcagtatcataactggcctccgctcactttcggcggagggaccaaggtggaaatcaaacgtacg

SEQ ID NO: 85 - protein, 124 aa, Homo sapiens
EVQLVESGGGLVQPGGSLRLSCVASTFSLINYWMTWVRQAPGKGLEWVANIKQDGFEKYYVDSVKGRFTISRDNAKNSLYLQMNSLRVEDTAVYYCARQGSYADTYFYHYGMDVWGQGTTVTVS

SEQ ID NO: 86 - DNA, 372 bp, Homo sapiens
gaggtgcagctggtggagtctgggggaggcttggtccagcctggggggtccctgagactctcctgtgtagcctccacgttcagccttattaactattggatgacctgggtccgccaggctcctgggaaggggctggagtgggtggccaacataaagcaggatggatttgagaaatactatgtggactctgtgaagggccgattcaccatctccagagacaacgccaagaattcactgtatctgcaaatgaacagcctgagagtcgaggacacggctgtgtattactgtgcgagacagggatcctacgctgacacgtacttctaccactacggtatggacgtctggggccaagggaccacggtcaccgtctcc

SEQ ID NO: 87 - protein, 234 aa, Homo sapiens [sequence omitted]

SEQ ID NO: 88 - DNA, 705 bp, Homo sapiens
atggcctggatgatgcttctcctcactctcctcgctcactgcacagggtcctgggcccagtctgcgctgacgcagccgccctcagtgtctggggccccagggcagagggtcaccatctcctgcactgggagcagctccaacatcggggcaggttatgatgcatactggtaccagcaacttccaggaacagcccccaaactcctcatctacggtaacagcaatcgactctcaggggtccctgaccgattctctgcctccaagtctggcacctcagcctccctggccatcactgggctccaggctgaggatgaggctgattattactgccagtcctatgacagcagcctgtcgattttcggcggagggaccaagctgaccgtcctacgtcagcccaaggctgccccctcggtcactctgttcccgccctcctctgaggagcttcaagccaacaaggccacactggtgtgtctcataagtgacttctacccgggagccgtgacagtggcctggaaggcagatagcagccccgtcaaggcgggagtggagaccaccacaccctccaaacaaagcaacaacaagtacgcggccagcagctacctgagcctgacgcctgagcagtggaagtcccacagaagctacagctgccaggtcacgcatgaagggagcaccgtggagaagacagtggcccctacagaatgttcatag

SEQ ID NO: 89 - protein, 124 aa, Homo sapiens
EVQLVESGGGLVQPGGSLRLSCAASGFTFNSYWMTWVRQAPGKGLEWVANINQDGSEKFFVDSVKGRFTISRDNAKTSLYLQMNSLRVEDTAVYYCAREGAPYDTYYYYYAMDVWGQGTTVTVS

SEQ ID NO: 90 - DNA, 372 bp, Homo sapiens
gaggtgcagctggtggagtctgggggaggcttggtccagcctggggggtccctgagactctcctgtgcagcctccggattcacctttaatagctattggatgacctgggtccgccaggctccagggaaggggctggagtgggtggccaacataaaccaagatggaagtgagaaattctttgtagactctgtgaagggccgattcaccatctccagagacaacgccaagacctcactgtatctgcaaatgaacagcctgagagtcgaggacacggctgtgtattactgtgcgagagagggggccccctatgatacatactactactactacgctatggacgtctggggccaagggaccacggtcaccgtctcc

SEQ ID NO: 91 - protein, 237 aa, Homo sapiens [sequence omitted]

SEQ ID NO: 92 - DNA, 714 bp, Homo sapiens
atggcctgggctctgctactcctcactctcctcgctcactgcacagggtcctgggcccagtctgtgctgacgcagccgccctcagtgtctggggccccagggcagagggtcaccatctcctgcactgggaccagctccaacatcggggcaggttatggtgtacactggtaccagcagcttccaggagcagcccccaaactcctcgtctatggtaacagcaatcggccctcaggggtccctgaccgattctctggctccaagtctggcacctcagcctccctggccatcactgggctccaggctgaggatgaggctgattattactgccagtcgtatgacatcagcctgagtggttcgaatgtcttcggaactgggaccaaggtcaccgtcctaggtcagcccaaggccaaccccactgtcactctgttcccgccctcctctgaggagctccaagccaacaaggccacactagtgtgtctgatcagtgacttctacccgggagctgtgacagtggcctggaaggcagatggcagccccgtcaaggcgggagtggagaccaccaaaccctccaaacagagcaacaacaagtacgcggccagcagctacctgagcctgacgcccgagcagtggaagtcccacagaagctacagctgccaggtcacgcatgaagggagcaccgtggagaagacagtggcccctacagaatgttcatag

SEQ ID NO: 93 - protein, 124 aa, Homo sapiens
EVQLVESGGGLVQPGGSLRLSCAASGFSFSSYWMTWVRQAPGKGLEWVANIKQYGSEKYYVDSVKGRFTISRDNAKNSLYLQMNSLRDDDTAVYYCARMGSYLDTYYYHYGMDVWGQGTTVTVS

SEQ ID NO: 94 - DNA, 372 bp, Homo sapiens
gaggtgcagctggtggagtctgggggaggcttggtccagcctggggggtccctgagactctcctgtgcagcgtctggattcagctttagtagctattggatgacctgggtccgccaggctccagggaaggggctggagtgggttgccaacataaagcaatatggaagtgagaaatactatgtggactctgtgaagggccgattcaccatctccagagacaacgccaagaactcactttatctacaaatgaacagcctaagagacgacgacacggccgtttattactgtgcgagaatggggagttatctagatacttactactaccactacggtatggacgtctggggccaagggaccacggtcaccgtctcc

SEQ ID NO: 95 - protein, 237 aa, Homo sapiens [sequence omitted]

SEQ ID NO: 96 - DNA, 714 bp, Homo sapiens
atggcctggatgatgcttctcctcactctcctcgctcactgcacagggtcctgggcccagtctgtgctgacgcagccgccctcactgtctggggccccagggcagagggtcaccatctcctgcacggggagcagctccaacatcggggcagattatgatgtatattggtatcagcaccttccaggaacagcccccaaactcctcatgtatggtgacggctatcggccctcaggggtccctgaccgattctctggctccaagtctggcacctcagcctccctggccatcactgggctccaggctgaggatgaggctgattattactgccagtcctatgacagcagtctgagtcgtcgtgtggtattcggcggagggaccaagctgaccgtccttggtcagcccaaggctgccccctcggtcactctgttcccgccctcctctgaggagcttcaagccaacaaggccacactgatgtgtctcataagtgacttctacccgggagccgtgacagtggcctggaaggcagatagcagccccgtcaaggcgggagtggagaccaccacaccctccaaacaaagcaacaacaagtacgcggccagcagctatctgagcctgacgcctgagcagtggaagtcccacagaagctacagctgccaggtcacgcatgaagggagcaccgtggagaagacagtggcccctacagaatgttcatag

SEQ ID NO: 97 - protein, 124 aa, Homo sapiens
EVQLVESGGGLVQPGGSLRLSCAASEFNFKSYWMTWVRQAPGKGLEWVANINQDGSEKNYVDSVKGRFTISRDNAKNSLHLQMSSLRVDDTAVYYCARTGSSWDTYYYYYAMDVWGQGTTVTVS

SEQ ID NO: 98 - DNA, 372 bp, Homo sapiens
gaggtgcagctggtggagtctgggggaggcttggtccagccgggggggtccctacgactctcctgtgcagcctctgaattcaactttaagagttattggatgacctgggtccgccaggctccagggaaggggctggagtgggtggccaacataaaccaagatggaagtgagaaaaactatgtggactctgtgaagggccgattcaccatctccagagataatgccaagaactcgctgcatctgcaaatgagcagcctgagagtcgacgacacggctgtatattactgtgcgcgaacgggcagcagttgggacacatactactactactacgctatggacgtctggggccaagggaccacggtcaccgtctcc

SEQ ID NO: 99 - protein, 107 aa, Homo sapiens
DIQLTQSPVSLSLSPGERATLSCRASQSVSSSYLVWYQQKPGQAPRLLIYGASSRAPGIPDRFSGSGSGTDFTLTISRLEREDFAVYYCQQYGRSFGQGTKVEIKRT

SEQ ID NO: 100 - DNA, 321 bp, Homo sapiens
gacatccagttgacccagtctccagtctccctgtctttgtctccaggggaaagagccaccctctcctgcagggccagtcagagtgttagcagcagctacttggtctggtaccagcagaaacctggccaggctcccaggctcctcatctatggtgcatccagcagggcccctggcatcccagacaggttcagtggcagtgggtctgggacagacttcactctcaccatcagcagactggagcgcgaagattttgcagtgtattattgtcagcagtatggtaggtcttttggccaggggaccaaggtggagatcaaacgtacg

SEQ ID NO: 101 - protein, 124 aa, Homo sapiens
EVQLVESGGGLVQPGGSLRLSCAASGFIFESYWMTWVRQAPGKGLEWVANIKQDGSEKNYVDSVKGRFTISRDNAKTSLYLQMNSLRAEDTAMYYCARSGSNWDTYYYYYGMDVWGQGTTVTVS

SEQ ID NO: 102 - DNA, 372 bp, Homo sapiens
gaggtgcagctggtggagtctgggggaggcttggtccagccgggggggtccctgagactctcctgtgcagcctctggattcatttttgaaagttactggatgacctgggtccgccaggctccagggaaggggctggaatgggtggccaacataaagcaagatggaagtgagaaaaactatgtggactctgtgaagggccgattcaccatctccagagacaacgccaagacctcactgtatctgcaaatgaacagcctgagagccgaggacacggccatgtattactgtgcgagatcgggcagcaactgggacacatattactactactacggtatggacgtctggggccaagggaccacggtcaccgtctcc

SEQ ID NO: 103 - protein, 107 aa, Homo sapiens
EIVLTQSPGTLSLSPGERATLSCRASQSVDSNYLGWYQQRPGQAPRLLIYGASSRATGVPDRFSASGSGTDFTLSISRLEPEDFAVYYCQQYGSSFGQGTKVEIKRT

SEQ ID NO: 104 - DNA, 321 bp, Homo sapiens
gaaattgtgttgacgcagtctccaggcaccctgtctttgtctccaggggaaagagccaccctctcctgcagggccagtcagagtgttgacagcaactacttaggctggtaccagcagagacctggccaggctcccaggctcctcatctatggtgcatccagcagggccactggcgtcccagacaggttcagtgccagtgggtctgggacagacttcactctcagcattagcagactggagcctgaagattttgcagtgtactactgtcagcagtatggtagctcttttggccaggggaccaaggtggaaatcaaacgtacg

SEQ ID NO: 105 - DNA, 32 bp, Artificial Sequence
  Synthetic primer
ccatcagctggggggtccctgagactctcctg

SEQ ID NO: 106 - DNA, 29 bp, Artificial Sequence
  Synthetic primer
cgctcagcttacctgaggagacggtgacc

SEQ ID NO: 107 - DNA, 50 bp, Artificial Sequence
  Synthetic primer
cgtatcgcctccctcgcgccatcagctggggggtccctgagactctcctg

SEQ ID NO: 108 - DNA, 47 bp, Artificial Sequence
  Synthetic primer
ctatgcgccttgccagcccgctcagcttacctgaggagacggtgacc

SEQ ID NO: 109 - protein, 8 aa, Artificial Sequence
  Linker sequence
GGGSGGGS

(19) United States
(12) Patent Application Publication
(10) Pub. No.: US 2013/0289246 A1
(43) Pub. Date: Oct. 31, 2013

(54) INFLUENZA VIRUS ANTIBODIES AND IMMUNOGENS AND USES THEREFOR

(71) Applicants: James E. Crowe, Nashville, TN (US); Jens C. Krause, Freiburg (DE); David L. Blum, Athens, GA (US)

(72) Inventors: James E. Crowe, Nashville, TN (US); Jens C. Krause, Freiburg (DE); David L. Blum, Athens, GA (US)

(73) Assignee: VANDERBILT UNIVERSITY, Nashville, TN (US)

(21) Appl. No.: 13/877,163

(22) PCT Filed: Sep. 30, 2011
(86) PCT No.: PCT/US11/54340

§ 371 Date: Jul. 18, 2013

Related U.S. Application Data
(60) Provisional application No. 61/388,393, filed on Sep. 30, 2010.

(51) Int. Cl. C07K 16/10

(52) U.S. Cl. CPC C07K 16/1018
USPC 530/381; 530/388.25; 530/387.3

(57) ABSTRACT

The present invention is directed to particular monoclonal antibodies and fragments thereof that find use in the detection, prevention and treatment of influenza virus infections. In particular, these antibodies may neutralize or limit the replication of influenza virus. Also disclosed are improved methods for producing such monoclonal antibodies, including novel immunogens for use in vaccination and production of protective immune responses.

This application claims benefit of priority to U.S. Provisional Application Ser. No. 61/388,393, filed Sep. 30, 2010, the entire contents of which are hereby incorporated by reference.

This invention was made with government support under grant number P01 AI058113 and Contract HHSN272200900047C awarded by the National Institutes of Health. The government has certain rights in the invention.

### BACKGROUND OF THE INVENTION

1. Field of the Invention

The present invention relates generally to the fields of virology, immunology and pathology. More particularly, it concerns the development of monoclonal antibodies and immunogens for use in the diagnosis, prevention and therapy of influenza virus infections.

2. Background of the Invention

Influenza, commonly referred to as the flu, is an infectious disease caused by RNA viruses of the family Orthomyxoviridae (the “influenza viruses”), that affects birds and mammals. The most common symptoms of the disease are chills, fever, pharyngitis, muscle pains, severe headache, coughing, weakness and general discomfort. Fever and coughs are the most frequent symptoms. In more serious cases, influenza causes pneumonia, which can be fatal, particularly for the young and the elderly. Although it is often confused with the common cold, influenza is a much more severe disease and is caused by a different type of virus. Influenza may produce nausea and vomiting, particularly in children, but these symptoms are more common in the unrelated disease gastroenteritis, which is sometimes called “stomach flu” or “24-hour flu.”

Typically, influenza is transmitted from infected mammals through the air by coughs or sneezes, creating aerosols containing the virus, and from infected birds through their droppings. Influenza can also be transmitted by saliva, nasal secretions, feces and blood. Infections also occur through contact with these body fluids or with contaminated surfaces. Flu viruses can remain infectious for about one week at human body temperature, over 30 days at 0° C. (32° F.), and for much longer periods at very low temperatures. Influenza viruses can be inactivated by disinfectants and detergents. As the virus can be inactivated by soap, frequent hand washing reduces the risk of infection.

The symptoms of human influenza were clearly described by Hippocrates roughly 2,400 years ago. Since then, the virus has caused numerous pandemics. Historical data on influenza are difficult to interpret, because the symptoms can be similar to those of other diseases, such as diphtheria, pneumonic plague, typhoid fever, dengue, or typhus. The first convincing record of an influenza pandemic was of an outbreak in 1580, which began in Russia and spread to Europe via Africa. In Rome, over 8,000 people were killed, and several Spanish cities were almost wiped out. Pandemics continued sporadically throughout the 17th and 18th centuries, with the pandemic of 1830-1833 being particularly widespread; it infected approximately a quarter of the people exposed. The most famous and lethal outbreak was the so-called Spanish flu pandemic (type A influenza, H1N1 subtype), which lasted from 1918 to 1919. It is not known exactly how many it killed, but estimates range from 20 to 100 million people. Later flu pandemics were not so devastating. They included the 1957 Asian Flu (type A, H2N2 strain) and the 1968 Hong Kong Flu (type A, H3N2 strain), but even these smaller outbreaks killed millions of people. In later pandemics, antibiotics were available to control secondary infections and this may have helped reduce mortality compared to the Spanish Flu of 1918. An avian strain named H5N1 has recently posed the greatest risk for a new influenza pandemic since it first killed humans in Asia in the 1990's. Each of these pandemics was caused by the appearance of a new strain of the virus in humans. Often, these new strains result from the spread of an existing flu virus to humans from other animal species.

Vaccinations against influenza are usually given to people in developed countries and to farmed poultry. The most common human vaccine is the trivalent influenza vaccine (TIV) that contains purified and inactivated material from three viral strains. Typically, this vaccine includes material from two influenza A virus subtypes and one influenza B virus strain. The TIV carries no risk of transmitting the disease, and it has very low reactivity. A vaccine formulated for one year may be ineffective in the following year, since the influenza virus evolves rapidly, and different strains become dominant. Antiviral drugs can be used to treat influenza, with neuraminidase inhibitors being particularly effective.

In April 2009, a novel H1N1 flu strain that combined genes from human, pig, and bird flu, emerged in Mexico, the United States, and several other nations. By late April, the virus was suspected of having killed over 150 in Mexico, prompting concern of a new pandemic. Its structural similarity to the pandemic 1918 Spanish Flu highlights the ongoing threat from influenza virus generally, and the H1N1 subtype in particular. Therefore, compositions and methods for the diagnosis, prevention and treatment of this disease remain highly sought after.

### SUMMARY OF THE INVENTION

Thus, in accordance with the present invention, there is provided a human monoclonal antibody that (a) binds to globular head region of influenza virus hemagglutinin; (b) cross-reacts between Group 1 and Group 2 influenza viruses; and (c) neutralizes virus and/or inhibits influenza hemagglutination. The antibody may further cross-react with multiple Group 1 influenza viruses, such as H1, H2, H5 and/or H9. It may also further cross-react with multiple Group 2 influenza viruses, such as H3 and/or H7. It may neutralize influenza virus and/or inhibit influenza hemagglutination. The antibody may be recombinant. It may bind the same epitope as 8F24, or the same epitope as 3E22 or 5I17. The heavy/light chain variable region sequences may be selected from the group consisting of SEQ ID NO:1/3, SEQ ID NO:5/7 and SEQ ID NO:9/11, including individual CDRs included therein.

In another embodiment, there is provided a human monoclonal antibody that binds to a long alpha helix region of influenza virus hemagglutinin and cross-reacts between multiple H1 influenza viruses. The antibody may further cross-react with multiple Group 1 influenza viruses and may be Group 1-specific, including H1, H2 and/or H5. The antibody may exhibit virus neutralization activity. The antibody may be a recombinant antibody. The antibody may further cross-react with multiple Group 1 influenza viruses and be Group 2-cross-reactive. The heavy/light chain variable region sequences may be selected from the group consisting of SEQ ID NO: 13/15, SEQ ID NO:29/31 and SEQ ID NO:33/35, including individual CDRs included therein.

In yet another embodiment, there is provided a human monoclonal antibody that binds to a long alpha helix region of influenza virus hemagglutinin and cross-reacts between multiple H3 influenza viruses. The antibody may further cross-react with multiple Group 2 influenza viruses and be Group 2-specific. The multiple Group 2 influenza viruses may comprise H3 and H7, H3 and H9, or H3, H7 and H9. The antibody may exhibit virus neutralization activity. The antibody may be a recombinant antibody. The heavy/light chain variable region sequences may be selected from the group consisting of 5J4 and 1C23, including CDRs included therein.

In still yet another embodiment, there is provided a human monoclonal antibody that binds to the stalk domain of the influenza virus hemagglutinin and cross-reacts between multiple H1 and multiple Group 1 influenza viruses, such as 8D4 and 19A14, or encoded by the heavy/light chain variable region sequences are selected from the group consisting of SEQ ID NO: 17/19, SEQ ID NO: 21/23 and SEQ ID NO: 25/27, including individual CDRs included therein. The multiple Group 1 influenza viruses may comprise H1 and H2, H1 and H5, or H1, H2 and H5. The antibody may Group 1-specific, or Group 2-cross-reactive.

In a further embodiment, there is provided an immunogen consisting essentially of (a) a long alpha helix (LAH) region of influenza virus hemagglutinin; and (b) a trimerization domain. The immunogen may further comprise a peptide purification tag, and/or a C-terminal cysteine residue, and/or a linker, such as GSA or SGR. The LAH region may be from a Group 1 influenza virus, such as H1, or from a Group 2 influenza virus, such as H3. The immunogen may further be linked to a carrier protein.

In still a further embodiment, there is provided a method of generating an immune response in a subject against a long alpha helix region of influenza virus hemagglutinin comprising administering to the subject an immunogen as described above. The immune response may be a protective immune response. The immune response may be a humoral response, such as a virus-neutralizing antibody response. The method may further comprise administering the immunogen to the subject a second time. The method may further comprise assessing an immune response to the immunogen by the subject following administration. The method may further comprise administering to the subject a second and distinct influenza virus antigen, such as an intact hemagglutinin stalk region, an intact hemagglutinin comprising a stalk and globular head region, or a seasonal flu vaccine.

Even further embodiments comprise:
a human monoclonal antibody that (a) binds to globular head region of influenza virus hemagglutinin; and (b) cross-reacts between early 20^{th} century H1 influenza viruses, and that may further bind to 2009 H1 influenza virus, and/or excludes antibody designated 2D1, such as the antibody designated 2K11, 2O10, 4A10, 4K8, 6D9, 5J8, 1F1, 1I20, 12D7, 4D20 or 2B12;
a human monoclonal antibody that (a) binds to globular head region of influenza virus hemagglutinin; and (b) binds to 1957 H2 influenza virus, and that may further bind to all H2 influenza viruses, such as the antibody designated 17J8, 18E6, 8G6, 25F7, 8F8, 2H22, 8M2, 2G1, 8K20, 4I4 or 4E2;
a human monoclonal antibody that (a) binds to globular head region of influenza virus hemagglutinin; and (b) binds to 1968 H3 influenza virus, and that may further bind to all H3 influenza viruses, such as the antibody designated 7A13, 2L15, 11J19 and 15C13;
a human monoclonal antibody that (a) binds to globular head region of influenza virus hemagglutinin; and (b) binds to Vietnam H5 influenza virus, and that further bind to all H5 influenza viruses, such as the antibody designated 13H19 or 4K4, or that may further be Vietnam H5-specific, such as the antibody designated 18D16, 2D16, 2M13 or 6N1; and
a human monoclonal antibody that (a) binds to globular head region of influenza virus hemagglutinin; and (b) cross-reacts between late 20^{th} century H1 influenza viruses, such as the antibody designated 3J10, 11K12, 12D7, 5I5, 12I1 or 2C7.

Even additional embodiments includes:
a method of protecting a subject from an influenza virus infection comprising administering to the subject an immunogen as described above or an antibody as described above;
a method of treating a subject having an influenza virus infection comprising administering to the subject an immunogen as described above or an antibody as described above;
a method of limiting an influenza virus infection in a subject comprising administering to the subject an immunogen as described above or an antibody as described above; or
a method of reducing the spread of influenza virus in a population comprising administering to one or more members of the population an immunogen as described above or an antibody as described above.

Also provided is a method of identifying a candidate protective antibody or antibody-producing cell comprising (a) providing an antibody- or B-cell containing sample; and (b) assessing for binding of an antibody in or produced by the sample with an immunogen as described above, wherein a positive reaction between the antibody and the immunogen identifies the antibody as candidate protective antibody.

Yet an additional embodiment comprises a method of identifying phylogenetically-related antibodies in a subject comprising (a) obtaining B-cells from said subject; (b) preparing hybridomas from said obtained B-cells; (c) assessing hybridomas for the production of neutralizing antibodies against a pathogen; (d) comparing antibody-coding sequences from hybridomas that produce neutralizing antibodies; (e) determining a common pattern of H chain usage, L chain usage and/or junctional sequences in neutralizing antibodies; and (f) determining the H chain usage, L chain usage and/or junctional sequences in B-cells from said subject, thereby identifying phylogenetically-related antibodies in said subject. Specifically contemplated are high throughput sequencing methods such as 454 sequencing, Ion Torrent semiconductor based sequencing and Illumina Solexa sequencing.

It is contemplated that any method or composition described herein can be implemented with respect to any other method or composition described herein.

The use of the word “a” or “an” when used in conjunction with the term “comprising” in the claims and/or the specification may mean “one,” but it is also consistent with the meaning of “one or more,” “at least one,” and “one or more than one.” The word “about” means plus or minus 5% of the stated number.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1. Phylogram based only on LAH sequences, for 16 flu HA subtypes. Group 1 and 2 LAH sequences diverge.

FIG. 2. Amino acid 76-130 HA2 LAH constructs with a trimerization domain.

FIG. 3. Influenza Ab repertoire of a 2009 H1N1 vaccinee. 93 flu-specific EBV lines from one donor were tested against a panel of flu antigens including HAs and LAHs. From a single donor, the inventors identified lines secreting antibodies to Group 1, or Group 2 or both Group 1 & 2 LAH, or non-LAH mAbs to all H1s, or to all Group 1 antigens (H1+H5).

FIG. 4. Antigenic regions on the head of the HA. In this case, antigenic regions of the 1918 HA can be identified by sequence homology with HA molecules for which epitope mapping has been performed with mouse or human mAbs (sites Sa, Sb, Ca1, Ca2, Cb). Position of escape mutations the inventors induced with human mAbs are indicated.

FIG. 5. Space-filling model of 1957 influenza HA (PDB: 3KU3) (Xu et al., 2010a); view onto the RBD of the membrane-distal globular head of a single monomer. Residues that mediate escape from mAbs when mutated are colored: red for 8F8 escape mutations, green for 8M2 escape mutations; blue for the 2G1 escape mutation. Other residues that are part of the RBD, but have not been implicated as escape mutations of 8F8 or 8M2 are colored in dark grey.

FIGS. 6A-B. Therapeutic efficacy of mAb 8F8, 8M2, 2G1, or a human IgG control against disease caused by the A/Albany/6/1958 H2N2 virus in mice. Mice were inoculated on day 0 and treated on day 1 with the indicated antibody and dose. In each group, six mice were monitored for survival (FIG. 6A) and weight (FIG. 6B). At the 8F8 200 μg dose (p<0.01), the 8F8 20 μg dose (p<0.05), the 8M2 200 μg dose (p<0.01), the 2G1 200 μg dose (p<0.01), and the 2G1 20 μg dose (p<0.01), treatment conferred a survival advantage by log-rank test.

FIG. 7. Phylogram of all 84 naturally-occurring, non-redundant human H2N2 HA sequences in the Influenza Research Database based on the protein sequences of residues 59-252 (“globular head” domain) of the HA1 subunit (MacVector 12.0.1). The phylogram branches are colored green for early H2 strains and blue for late H2 strains.

FIG. 8A-D. Multiple sequence alignment of HA amino acid residues 59-252 (“globular head”) of all 84 naturally-occurring, non-redundant human H2N2 strains in the Influenza Research Database in the order of the phylogram (FIG. 7). Residues that belong to the RBD are highlighted in red in the A/Japan/305/1957 (CY014976) sequence at the top. Residues identical to those of this Japan/305 strain are denoted by solid color, green for early H2 strains and blue for late H2 strains. Mutated residues are highlighted in white (or grey for conservative mutations). Residues that have been identified as contact residues of the human H2 mAbs 8F8, 8M2, or 2G1 are annotated with the mAb name in grey at the top.

FIGS. 9A-B. Therapeutic efficacy of mAb 5J8 against disease caused by the 1918 A(H1N1) virus in mice. Mice were inoculated on day 0 and treated on day 1 with the indicated antibody and dose. In each group, six mice were monitored every other day for survival (FIG. 9A) and weight (FIG. 9B). At the high-dose level and the intermediate-dose level, the differences in survival distribution between the mAb 5J8 and the human IgG control groups were significant by log-rank test (p=0.001 for the high-dose level, p<0.001 for the intermediate-dose level, p=0.238 for the low-dose level).

FIG. 10. Space-filling model of 1918 influenza HA (PDB: 1RD8) (Stevens et al., 2004); view onto the membrane-distal globular head. The three HA monomer subunits are colored in white, gray, or black. The conventionally-defined antigenic sites on HA are colored blue (site Sa), yellow (site Sb), or green (site Ca₂). MAb 5J8 selected for mutations in residues 133A, 137, 199, or 222 in certain H1N1 viruses (magenta). These residues are situated between the receptor-binding pocket and the Ca₂ antigenic site, but are themselves not part of a conventionally-defined antigenic site.

FIGS. 11A-B. Therapeutic efficacy of Ab 4K8 against disease caused by the 1918 A(H1N1) virus in mice. Mice were inoculated on day 0 and treated on day 1 with the indicated antibody and dose. In each group, six mice were monitored every other day for survival (FIG. 11A) and weight (FIG. 11B). At all dose levels, the differences in survival distribution between the 4K8 and the human IgG control groups were significant by log-rank test (p=0.001 for the high-dose level, p<0.001 for the medium-dose level, p<0.01 for the low-dose level).

FIG. 12. Comparison of antibody gene junctional sequences reveals four independent clones. The IgH gene segment junctions of the five V_{H}3-7/J_{H}6 antibodies 4A10, 2O10, 4K8, 6D9, and 2K11 are shown in amino acid and DNA sequence. Mutated amino acids and nucleotides are underlined. The amino acid residues are color-coded per standard IMGT color scheme based on chemical properties (Pommie et al., 2004). Briefly, aliphatic (A, I, L, V) residues are dark blue, phenylalanine light blue, sulfur (C, M) residues cyan, glycine dark green, residues with hydroxyl groups (S, T) medium green, tryptophan pink, tyrosine light green, proline yellow, acidic (D, E) residues light orange, amide (N, Q) residues dark orange, and basic (H, K, R) residues red. Kabat numbering for amino acids is used instead of IMGT numbering, and is shown at the top level; the CDR H3 margins are denoted in red. The contributions of the V_{H}, D, and J_{H} genes are shown in light grey (V_{H}), medium grey (D), and dark grey (J_{H}).

FIG. 13. Phylogram and sequence alignment to the V_{H}3-7*01 germline sequence of the heavy variable chain genes of Abs 4A10 (cyan), 2010 (orange), 4K8/6D9 (medium blue), and 2K11 (green) from hybridoma technology and pyrosequencing. Five-letter alphanumeric labels denote sequences derived from pyrosequencing; hybridoma names are italicized. The location of the CDRs (based on IMGT analysis) is shown on top; Kabat numbering is shown at the bottom. Amino acids similar to the V_{H}3-7*01 germline sequence (for the V_{H} region) or to the consensus sequence (for the D/J regions) are in light gray, dissimilar amino acids in white. Variable gene segment (V-GENE) encoded sequences are separated from the diversity and joining gene segment (D/J-GENE) encoded sequences by a dashed line. Residues within the V_{H} region with evidence of convergence are identified with an asterisk at the bottom. The phylogram was generated based on the protein sequences with MacVector software version 12 using neighbor joining, best tree, symmetric tie breaking, uncorrected (“p”) distance settings, and rooted to the deduced V_{H}3-7*01 germ line protein sequence.

### DESCRIPTION OF ILLUSTRATIVE EMBODIMENTS

As discussed above, influenza virus is the leading viral cause of severe respiratory tract illness in persons of all ages, and can also cause severe illness and death in the very young and elderly. Some particularly lethal strains can be fatal to even healthy young adults. All of these patient groups would benefit from more effective antiviral therapeutic options for influenza virus, and in particular, the subtypes responsible for previous and future pandemic outbreaks.

The present invention provides new monoclonal antibodies that can be delivered in the same manner as currently approved anti-viral therapies. The antibodies bind to the virus and prevent the virus from infecting a cell. The antibodies also can be used prophylactically as vaccines, and diagnostically. In addition, new immunogenic compositions derived from the hemagglutinin molecule are provided and proposed for use in generating monoclonal antibodies as well as in traditional vaccines. These and other aspects of the invention are described in detail below.

### II. INFLUENZA VIRUS

A. General

The etiological cause of influenza, the Orthomyxoviridae family of viruses, was first discovered in pigs by Richard Shope in 1931. This discovery was shortly followed by the isolation of the virus from humans by a group headed by Patrick Laidlaw at the Medical Research Council of the United Kingdom in 1933. However, it was not until Wendell Stanley first crystallized tobacco mosaic virus in 1935 that the non-cellular nature of viruses was appreciated.

The first significant step towards preventing influenza was the development in 1944 of a killed-virus vaccine for influenza by Thomas Francis, Jr. This built on work by Australian Frank Macfarlane Burnet, who showed that the virus lost virulence when it was cultured in fertilized hen's eggs. Application of this observation by Francis allowed his group of researchers at the University of Michigan to develop the first influenza vaccine, with support from the U.S. Army. The Army was deeply involved in this research due to its experience of influenza in World War I when thousands of troops were killed by the virus in a matter of months.

Although there were scares in the State of New Jersey in 1976 (with the Swine Flu), worldwide in 1977 (with the Russian Flu), and in Hong Kong and other Asian countries in 1997 (with H5N1 avian influenza), there have been no major pandemics since the 1968 Hong Kong Flu. Immunity to previous pandemic influenza strains and vaccination may have limited the spread of the virus and may have helped prevent further pandemics.

The influenza virus is an RNA virus of the family Orthomyxoviridae which comprises five genera: Influenzavirus A, Influenzavirus B, Influenzavirus C, Isavirus and Thogotovirus. The Influenzavirus A genus has one species, influenza A virus. Wild aquatic birds are the natural hosts for a large variety of influenza A. Occasionally, viruses are transmitted to other species and may then cause devastating outbreaks in domestic poultry or give rise to human influenza pandemics. The type A viruses are the most virulent human pathogens among the three influenza types and cause the most severe disease. The influenza A virus can be subdivided into different subtypes based on the antibody response to these viruses. The subtypes that have been confirmed in humans, ordered by the number of known human pandemic deaths, are:
H1N1, which caused Spanish flu in 1918 and has been identified as the subtype of the 2009 outbreak of swine flu originating from Mexico
H2N2, which caused Asian Flu in 1957
H3N2, which caused Hong Kong Flu in 1968
H5N1, a pandemic threat in the 2007-08 flu season
H7N7, which has unusual zoonotic potential
H1N2, endemic in humans and pigs
H9N2
H7N2
H7N3
H10N7

Influenzaviruses A, B and C are very similar in structure. The virus particle is 80-120 nanometres in diameter and usually roughly spherical, although filamentous forms can occur. This particle is made of a viral envelope containing two main types of glycoproteins, wrapped around a central core. The central core contains the viral RNA genome and other viral proteins that package and protect this RNA. Unusually for a virus, its genome is not a single piece of nucleic acid; instead, it contains seven or eight pieces of segmented negative-sense RNA. The Influenza A genome encodes 11 proteins: hemagglutinin (HA), neuraminidase (NA), nucleoprotein (NP), M1, M2, NS1, NS2(NEP), PA, PB1, PB1-F2 and PB2.

Hemagglutinin (HA) and neuraminidase (NA) are the two large glycoproteins on the outside of the viral particles. HA is a lectin that mediates binding of the virus to target cells and entry of the viral genome into the target cell, while NA is involved in the release of progeny virus from infected cells, by cleaving sugars that bind the mature viral particles. Thus, these proteins are targets for antiviral drugs. Furthermore, they are antigens to which antibodies can be raised. Influenza A viruses are classified into subtypes based on antibody responses to HA and NA. These different types of HA and NA form the basis of the H and N distinctions in, for example, H5N1.

Influenza viruses bind through hemagglutinin onto sialic acid sugars on the surfaces of epithelial cells typically in the nose, throat and lungs of mammals and intestines of birds. The cell imports the virus by endocytosis. In the acidic endosome, part of the hemagglutinin protein fuses the viral envelope with the vacuole's membrane, releasing the viral RNA (vRNA) molecules, accessory proteins and RNA-dependent RNA polymerase into the cytoplasm. These proteins and vRNA form a complex that is transported into the cell nucleus, where the RNA-dependent RNA polymerase begins transcribing complementary positive-sense vRNA. The vRNA is either exported into the cytoplasm and translated, or remains in the nucleus. Newly-synthesised viral proteins are either secreted through the Golgi apparatus onto the cell surface or transported back into the nucleus to bind vRNA and form new viral genome particles. Other viral proteins have multiple actions in the host cell, including degrading cellular mRNA and using the released nucleotides for vRNA synthesis and also inhibiting translation of host-cell mRNAs.

Negative-sense vRNAs that form the genomes of future viruses, RNA-dependent RNA polymerase, and other viral proteins are assembled into a virion. Hemagglutinin and neuraminidase molecules cluster into a bulge in the cell membrane. The vRNA and viral core proteins leave the nucleus and enter this membrane protrusion. The mature virus buds off from the cell in a sphere of host phospholipid membrane, acquiring hemagglutinin and neuraminidase with this membrane coat. As before, the viruses adhere to the cell through hemagglutinin; the mature viruses detach once their neuraminidase has cleaved sialic acid residues from the host cell. After the release of new influenza viruses, the host cell dies.

Because of the absence of RNA proofreading enzymes, the RNA-dependent RNA polymerase makes a single nucleotide insertion error roughly every 10 thousand nucleotides, which is the approximate length of the influenza vRNA. Hence, the majority of newly-manufactured influenza viruses are mutants, causing “antigenic drift.” The separation of the genome into eight separate segments of vRNA allows mixing or reassortment of vRNAs if more than one viral line has infected a single cell. The resulting rapid change in viral genetics produces antigenic shifts and allows the virus to infect new host species and quickly overcome protective immunity.

B. The 1918 “Spanish” Flu

The 1918 flu pandemic, commonly referred to as the Spanish Flu, was an influenza pandemic that spread to nearly every part of the world. It was caused by an unusually virulent and deadly Influenza A virus strain of subtype H1N1. Historical and epidemiological data are inadequate to identify the geographic origin of the virus. Most of its victims were healthy young adults, in contrast to most influenza outbreaks which predominantly affect juvenile, elderly, or otherwise weakened patients. The pandemic lasted from March 1918 to June 1920, spreading even to the Arctic and remote Pacific islands. It is estimated that anywhere from 20 to 100 million people were killed worldwide, or the approximate equivalent of one third of the population of Europe, more than double the number killed in World War I. This extraordinary toll resulted from the extremely high illness rate of up to 50% and the extreme severity of the symptoms, suspected to be caused by cytokine “storms.” The pandemic is estimated to have affected up to one billion people—half the world's population at the time.

Scientists have used tissue samples from frozen victims to reproduce the virus for study. Among the conclusions of this research is that the virus kills via a cytokine storm, an overreaction of the body's immune system, which explains its unusually severe nature and the concentrated age profile of its victims. The strong immune systems of young adults ravaged the body, whereas the weaker immune systems of children and middle-aged adults caused fewer deaths.

The global mortality rate from the 1918/1919 pandemic is not known, but is estimated at 2.5 to 5% of those who were infected died. Note this does not mean that 2.5-5% of the human population died; with 20% or more of the world population suffering from the disease to some extent, a case-fatality ratio this high would mean that about 0.5-1% (≈50 million) of the whole population died. Influenza may have killed as many as 25 million in its first 25 weeks. Older estimates say it killed 40-50 million people while current estimates say 50 million to 100 million people worldwide were killed. This pandemic has been described as “the greatest medical holocaust in history” and may have killed more people than the Black Death.

An effort to recreate the 1918 flu strain (a subtype of avian strain H1N1) was a collaboration among the Armed Forces Institute of Pathology, Southeast Poultry Research Laboratory and Mount Sinai School of Medicine in New York; the effort resulted in the announcement (on Oct. 5, 2005) that the group had successfully determined the virus' genetic sequence, using historic tissue samples recovered by pathologist Johan Hultin from a female flu victim buried in the Alaskan permafrost and samples preserved from American soldiers.

Kobasa et al. (2007) reported that monkeys (Macaca fascicularis) infected with the recreated strain exhibited classic symptoms of the 1918 pandemic and died from a cytokine storm—an overreaction of the immune system. This may explain why the 1918 flu had its surprising effect on younger, healthier people, as a person with a stronger immune system would potentially have a stronger overreaction. In December, 2008 research by Yoshihiro Kawaoka of University of Wisconsin linked the presence of three specific genes (termed PA, PB1, and PB2) and a nucleoprotein derived from 1918 flu samples to the ability of the flu virus to invade the lungs and cause pneumonia. The combination triggered similar symptoms in animal testing.

C. The 2009 “Swine” Flu

The 2009 flu pandemic was a global outbreak of a new strain of H1N1 influenza virus, often referred to as “swine flu.” The virus was first detected in April 2009 and contains a combination of genes from swine, avian (bird), and human influenza viruses. The outbreak began in the state of Veracruz, Mexico, with evidence that there had been an ongoing epidemic for months before it was officially recognized as such. The Mexican government closed most of Mexico City's public and private facilities in an attempt to contain the spread of the virus. However the virus continued to spread globally, clinics in some areas were overwhelmed by people infected, and the World Health Organization (WHO) and US Centers for Disease Control (CDC) stopped counting cases and in June declared the outbreak to be a pandemic.

While only mild symptoms are experienced by the majority of people, some have more severe symptoms. Mild symptoms may include fever, sore throat, cough, headache, muscle or joint pains, and nausea, vomiting, or diarrhea. Those at risk of a more severe infection include: asthmatics, diabetics, those with obesity, heart disease, the immunocompromised, children with neurodevelopmental conditions, and pregnant women. In addition, even for persons previously very healthy, a small percentage of patients will develop viral pneumonia or acute respiratory distress syndrome. This manifests itself as increased breathing difficulty and typically occurs 3-6 days after initial onset of flu symptoms.

Similar to other influenza viruses, pandemic H1N1 is typically contracted by person to person transmission through respiratory droplets. Symptoms usually last 4-6 days. Those with more severe symptoms or those in an at risk group may benefit from antivirals (oseltamivir or zanamivir). The CDC estimates that, in the United States alone, as of Nov. 14, 2009, there had been 9,820 deaths (range 7,070-13,930) caused by swine flu. Currently, there are almost 15,000 confirmed deaths worldwide.

D. Diagnosis and Treatments

Symptoms of influenza can start quite suddenly one to two days after infection. Usually the first symptoms are chills or a chilly sensation, but fever is also common early in the infection, with body temperatures ranging from 38-39° C. (approximately 100-103° F.). Many people are so ill that they are confined to bed for several days, with aches and pains throughout their bodies, which are worse in their backs and legs. Symptoms of influenza may include:
Body aches, especially joints and throat
Extreme coldness and fever
Fatigue
Headache
Irritated watering eyes
Reddened eyes, skin (especially face), mouth, throat and nose
Abdominal pain (in children with influenza B)
   It can be difficult to distinguish between the common cold and influenza in the early stages of these infections, but a flu can be identified by a high fever with a sudden onset and extreme fatigue. Diarrhea is not normally a symptom of influenza in adults, although it has been seen in some human cases of the H5N1 “bird flu” and can be a symptom in children.

Since antiviral drugs are effective in treating influenza if given early, it can be important to identify cases early. Of the symptoms listed above, the combinations of fever with cough, sore throat and/or nasal conjection can improve diagnostic accuracy. Two decision analysis studies suggest that during local outbreaks of influenza, the prevalence will be over 70%, and thus patients with any of these combinations of symptoms may be treated with neuramidase inhibitors without testing. Even in the absence of a local outbreak, treatment may be justified in the elderly during the influenza season as long as the prevalence is over 15%.

The available laboratory tests for influenza continue to improve. The United States Centers for Disease Control and Prevention (CDC) maintains an up-to-date summary of available laboratory tests. According to the CDC, rapid diagnostic tests have a sensitivity of 70-75% and specificity of 90-95% when compared with viral culture. These tests may be especially useful during the influenza season (prevalence=25%) but in the absence of a local outbreak, or peri-influenza season (prevalence=10%).

Influenza's effects are generally much more severe and last longer than those of the common cold. Most people will recover in about one to two weeks, but others will develop life-threatening complications (such as pneumonia). Influenza, however, can be deadly, especially for the weak, old or chronically ill. The flu can worsen chronic health problems. People with emphysema, chronic bronchitis or asthma may experience shortness of breath while they have the flu, and influenza may cause worsening of coronary heart disease or congestive heart failure. Smoking is another risk factor associated with more serious disease and increased mortality from influenza.

According to the World Health Organization, “Every winter, tens of millions of people get the flu. Most are only ill and out of work for a week, yet the elderly are at a higher risk of death from the illness. It is known that the worldwide death toll exceeds a few hundred thousand people a year, but even in developed countries the numbers are uncertain, because medical authorities don't usually verify who actually died of influenza and who died of a flu-like illness.” Even healthy people can be affected, and serious problems from influenza can happen at any age. People over 50 years old, very young children and people of any age with chronic medical conditions are more likely to get complications from influenza, such as pneumonia, bronchitis, sinus, and ear infections.

Common symptoms of the flu such as fever, headaches, and fatigue come from the huge amounts of proinflammatory cytokines and chemokines (such as interferon or tumor necrosis factor) produced from influenza-infected cells. In contrast to the rhinovirus that causes the common cold, influenza does cause tissue damage, so symptoms are not entirely due to the inflammatory response. This massive immune response can produce a life-threatening cytokine storm. This effect has been proposed to be the cause of the unusual lethality of both the H5N1 avian influenza, and the 1918 pandemic strain (see above).

In some cases, an autoimmune response to an influenza infection may contribute to the development of Guillain-Barré syndrome. However, as many other infections can increase the risk of this disease, influenza may only be an important cause during epidemics. This syndrome can also be a rare side-effect of influenza vaccines, with an incidence of about one case per million vaccinations.

People with the flu are advised to get plenty of rest, drink plenty of liquids, avoid using alcohol and tobacco and, if necessary, take medications such as paracetamol (acetaminophen) to relieve the fever and muscle aches associated with the flu. Children and teenagers with flu symptoms (particularly fever) should avoid taking aspirin during an influenza infection (especially influenza type B), because doing so can lead to Reye's syndrome, a rare but potentially fatal disease of the liver. Since influenza is caused by a virus, antibiotics have no effect on the infection; unless prescribed for secondary infections such as bacterial pneumonia, they may lead to resistant bacteria. Antiviral medication can be effective (see below), but some strains of influenza can show resistance to the standard antiviral drugs.

E. Influenza Virus Immunogens

Influenza hemagglutinin (HA) is an antigenic glycoprotein responsible for binding the virus to the cell that is being infected. There are 16 defined HA antigens. These subtypes are named H1 through H16. The last, H16, was discovered only recently on influenza A viruses isolated from black-headed gulls from Sweden and Norway. The first three hemagglutinins, H1, H2, and H3, are found in human influenza viruses.

HA has two functions. Firstly, it allows the recognition of target vertebrate cells, accomplished through the binding of these cells' sialic acid-containing receptors. Secondly, once bound it facilitates the entry of the viral genome into the target cells by causing the fusion of host endosomal membrane with the viral membrane. HA binds to the monosaccharide sialic acid which is present on the surface of its target cells, which causes the viral particles to stick to the cell's surface. The cell membrane then engulfs the virus and the portion of the membrane that encloses it pinches off to form a new membrane-bound compartment within the cell called an endosome, which contains the engulfed virus. The cell then attempts to begin digesting the contents of the endosome by acidifying its interior and transforming it into a lysosome. However, as soon as the pH within the endosome drops to about 6.0, the original folded structure of the HA molecule becomes unstable, causing it to partially unfold, and releasing a very hydrophobic portion of its peptide chain that was previously hidden within the protein. This so-called “fusion peptide” inserts itself into the endosomal membrane. Then, when the rest of the HA molecule refolds into a new structure (which is more stable at the lower pH), it pulls the endosomal membrane next to the virus particle's own membrane, causing the two to fuse together. Once this has happened, the contents of the virus, including its RNA genome, are free to pour out into the cell's cytoplasm.

HA is a homotrimeric integral membrane glycoprotein. It is shaped like a cylinder, and is approximately 13.5 nanometres long. The three identical monomers that constitute HA are constructed into a central α helix coil; three spherical heads contain the sialic acid binding sites. HA monomers are synthesized as precursors that are then glycosylated and cleaved into two smaller polypeptides: the HA1 and HA2 subunits. Each HA monomer consists of a long, helical chain anchored in the membrane by HA2 and topped by a large HA1 globule.

In one aspect, the present invention provides a new HA immunogen for use in generating useful antibodies and also as a vaccine. The immunogen is derived from the stalk region of influenza virus HA and consists essentially of the long alpha helix (LAH) region of this molecule. It also includes a trimerization domain. The “consists essentially of” in this context means that there are not any other influenza virus hemagglutinin sequences included in the immunogen that are sufficient to generate antibodies.

The immunogen may contain other elements, such as a peptide purification tag, a C-terminal cysteine residue, such as for linking to a carrier protein, and one or more linkers for assembling various components of the immunogen (e.g., GSA or SGR). The LAH region may be from a Group 1 or 2 virus. The following are exemplary sequences for such immunogens:

(SEQ ID NO: 61)
METDTLLLWVLLLWVPGSWARMENLNKKVDDGFLDIWTYNAELLVLLENE
RTLDFHDSNVKNLYEKVKSQLKNNASGRLVPRGSPGSGYIPEAPRDGQAY
VRKDGEWVLLSTFLGSAWSHPQFEK
(SEQ ID NO: 62)
METDTLLLWVLLLWVPGSWARIQDLEKYVEDTKIDLWSYNAELLVALENQ
HTIDLTDSEMNKLFEKTRRQLRENASGRLVPRGSPGSGYIPEAPRDGQAY
VRKDGEWVLLSTFLGSAWSHPQFEK
(SEQ ID NO: 63)
METDTLLLWVLLLWVPGSWARMENLNKKVDDGFLDIWTYNAELLVLLENE
RTLDFHDSNVKNLYEKVKSQLKNMKQIEDKIEEILSKIYHIENEIARIKK
LIGEAGSAWSHPQFEKGGGSGGGSGGSAWSHPQFEKGGGSGGGSC
(SEQ ID NO: 64)
METDTLLLWVLLLWVPGSWARIQDLEKYVEDTKIDLWSYNAELLVALENQ
HTIDLTDSEMNKLFEKTRRQLREMKQIEDKIEEILSKIYHIENEIARIKK
LIGEAGSAWSHPQFEKGGGSGGGSGGSAWSHPQFEKGGGSGGGSC

### III. PRODUCING MONOCLONAL ANTIBODIES

A. General Methods

It will be understood that monoclonal antibodies binding to influenza virus and related proteins will have utilities in several applications. These include the production of diagnostic kits for use in detecting and diagnosing disease. In these contexts, one may link such antibodies to diagnostic or therapeutic agents, or use them as capture agents or competitors in competitive assays. Means for preparing and characterizing antibodies are well known in the art (see, e.g., Antibodies: A Laboratory Manual, Cold Spring Harbor Laboratory, 1988; U.S. Pat. No. 4,196,265).

The methods for generating monoclonal antibodies (MAbs) generally begin along the same lines as those for preparing polyclonal antibodies. The first step for both these methods is immunization of an appropriate host or identification of subjects who are immune due to prior natural infection. As is well known in the art, a given composition for immunization may vary in its immunogenicity. It is often necessary therefore to boost the host immune system, as may be achieved by coupling a peptide or polypeptide immunogen to a carrier. Exemplary and preferred carriers are keyhole limpet hemocyanin (KLH) and bovine serum albumin (BSA). Other albumins such as ovalbumin, mouse serum albumin or rabbit serum albumin can also be used as carriers. Means for conjugating a polypeptide to a carrier protein are well known in the art and include glutaraldehyde, m-maleimidobencoyl-N-hydroxysuccinimide ester, carbodiimyde and bis-biazotized benzidine. As also is well known in the art, the immunogenicity of a particular immunogen composition can be enhanced by the use of non-specific stimulators of the immune response, known as adjuvants. Exemplary and preferred adjuvants include complete Freund's adjuvant (a non-specific stimulator of the immune response containing killed Mycobacterium tuberculosis), incomplete Freund's adjuvants and aluminum hydroxide adjuvant.

The amount of immunogen composition used in the production of polyclonal antibodies varies upon the nature of the immunogen as well as the animal used for immunization. A variety of routes can be used to administer the immunogen (subcutaneous, intramuscular, intradermal, intravenous and intraperitoneal). The production of polyclonal antibodies may be monitored by sampling blood of the immunized animal at various points following immunization. A second, booster injection, also may be given. The process of boosting and titering is repeated until a suitable titer is achieved. When a desired level of immunogenicity is obtained, the immunized animal can be bled and the serum isolated and stored, and/or the animal can be used to generate MAbs.

In the case of human monoclonal antibodies, one may instead simply look for an individual already known to have generated an immune response, in this case, to have been infected with influenza virus. Virtually all adults have significant serum titers of neutralizing antibodies to seasonal influenza viruses, therefore most adults are candidates to donate blood containing B cells from which the inventors' method can generate human monoclonal antibodies. In order to identify subjects with immunity to various influenza strain, one could generally obtain blood from subjects who were born prior to a given epidemic/pandemic, and who were likely to have been exposed. Many antibodies described in this invention were generated in this way using peripheral blood from otherwise healthy individuals previously infected with influenza viruses.

Following immunization or obtaining of cells from previously infected subjects as described above, somatic cells with the potential for producing antibodies, specifically B lymphocytes (B cells), are selected for use in the MAb generating protocol. These cells may be obtained from biopsied spleens or lymph nodes, or from circulating blood. The antibody-producing B lymphocytes from the immunized animal are then fused with cells of an immortal myeloma cell, generally one of the same species as the animal that was immunized or human or human/mouse chimeric cells. Myeloma cell lines suited for use in hybridoma-producing fusion procedures preferably are non-antibody-producing, have high fusion efficiency, and enzyme deficiencies that render then incapable of growing in certain selective media which support the growth of only the desired fused cells (hybridomas).

Any one of a number of myeloma cells may be used, as are known to those of skill in the art (Goding, pp. 65-66, 1986; Campbell, pp. 75-83, 1984). For example, where the immunized animal is a mouse, one may use P3-X63/Ag8, X63-Ag8.653, NS1/1.Ag 4 1, Sp210-Ag14, FO, NSO/U, MPC-11, MPC11-X45-GTG 1.7 and S194/5XX0 Bul; for rats, one may use R210.RCY3, Y3-Ag 1.2.3, IR983F and 4B210; and U-266, GM1500-GRG2, LICR-LON-HMy2 and UC729-6 are all useful in connection with human cell fusions. One particular murine myeloma cell is the NS-1 myeloma cell line (also termed P3-NS-1-Ag4-1), which is readily available from the NIGMS Human Genetic Mutant Cell Repository by requesting cell line repository number GM3573. Another mouse myeloma cell line that may be used is the 8-azaguanine-resistant mouse murine myeloma SP2/0 non-producer cell line. More recently, additional fusion partner lines for use with human B cells have been described, including KR12 (ATCC CRL-8658; K6H6/B5 (ATCC CRL-1823 SHM-D33 (ATCC CRL-1668) and HMMA2.5 (Posner et al., 1987). The antibodies in this invention were generated using the HMMA2.5 line.

Methods for generating hybrids of antibody-producing spleen or lymph node cells and myeloma cells usually comprise mixing somatic cells with myeloma cells in a 2:1 proportion, though the proportion may vary from about 20:1 to about 1:1, respectively, in the presence of an agent or agents (chemical or electrical) that promote the fusion of cell membranes. Fusion methods using Sendai virus have been described by Kohler and Milstein (1975; 1976), and those using polyethylene glycol (PEG), such as 37% (v/v) PEG, by Gefter et al. (1977). The use of electrically induced fusion methods also is appropriate (Goding, pp. 71-74, 1986). The hybridomas secreting the influenza antibodies in this invention were obtained by electrofusion.

Fusion procedures usually produce viable hybrids at low frequencies, about 1×10⁻⁶ to 1×10⁻⁸. However, this does not pose a problem, as the viable, fused hybrids are differentiated from the parental, infused cells (particularly the infused myeloma cells that would normally continue to divide indefinitely) by culturing in a selective medium. The selective medium is generally one that contains an agent that blocks the de novo synthesis of nucleotides in the tissue culture media. Exemplary and preferred agents are aminopterin, methotrexate, and azaserine. Aminopterin and methotrexate block de novo synthesis of both purines and pyrimidines, whereas azaserine blocks only purine synthesis. Where aminopterin or methotrexate is used, the media is supplemented with hypoxanthine and thymidine as a source of nucleotides (HAT medium). Where azaserine is used, the media is supplemented with hypoxanthine. Ouabain is added if the B cell source is an Epstein Barr virus (EBV) transformed human B cell line, in order to eliminate EBV transformed lines that have not fused to the myeloma.

The preferred selection medium is HAT or HAT with ouabain. Only cells capable of operating nucleotide salvage pathways are able to survive in HAT medium. The myeloma cells are defective in key enzymes of the salvage pathway, e.g., hypoxanthine phosphoribosyl transferase (HPRT), and they cannot survive. The B cells can operate this pathway, but they have a limited life span in culture and generally die within about two weeks. Therefore, the only cells that can survive in the selective media are those hybrids formed from myeloma and B cells. When the source of B cells used for fusion is a line of EBV-transformed B cells, as here, ouabain is also used for drug selection of hybrids as EBV-transformed B cells are susceptible to drug killing, whereas the myeloma partner used is chosen to be ouabain resistant.

Culturing provides a population of hybridomas from which specific hybridomas are selected. Typically, selection of hybridomas is performed by culturing the cells by single-clone dilution in microtiter plates, followed by testing the individual clonal supernatants (after about two to three weeks) for the desired reactivity. The assay should be sensitive, simple and rapid, such as radioimmunoassays, enzyme immunoassays, cytotoxicity assays, plaque assays dot immunobinding assays, and the like.

The selected hybridomas are then serially diluted or single-cell sorted by flow cytometric sorting and cloned into individual antibody-producing cell lines, which clones can then be propagated indefinitely to provide mAbs. The cell lines may be exploited for MAb production in two basic ways. A sample of the hybridoma can be injected (often into the peritoneal cavity) into an animal (e.g., a mouse). Optionally, the animals are primed with a hydrocarbon, especially oils such as pristane (tetramethylpentadecane) prior to injection. When human hybridomas are used in this way, it is optimal to inject immunocompromised mice, such as SCID mice, to prevent tumor rejection. The injected animal develops tumors secreting the specific monoclonal antibody produced by the fused cell hybrid. The body fluids of the animal, such as serum or ascites fluid, can then be tapped to provide MAbs in high concentration. The individual cell lines could also be cultured in vitro, where the MAbs are naturally secreted into the culture medium from which they can be readily obtained in high concentrations. Alternatively, human hybridoma cells lines can be used in vitro to produce immunoglobulins in cell supernatant. The cell lines can be adapted for growth in serum-free medium to optimize the ability to recover human monoclonal immunoglobulins of high purity.

MAbs produced by either means may be further purified, if desired, using filtration, centrifugation and various chromatographic methods such as FPLC or affinity chromatography. Fragments of the monoclonal antibodies of the invention can be obtained from the purified monoclonal antibodies by methods which include digestion with enzymes, such as pepsin or papain, and/or by cleavage of disulfide bonds by chemical reduction. Alternatively, monoclonal antibody fragments encompassed by the present invention can be synthesized using an automated peptide synthesizer.

It also is contemplated that a molecular cloning approach may be used to generate monoclonals. For this, RNA can be isolated from the hybridoma line and the antibody genes obtained by RT-PCR and cloned into an immunoglobulin expression vector. Alternatively, combinatorial immunoglobulin phagemid libraries are prepared from RNA isolated from the cell lines and phagemids expressing appropriate antibodies are selected by panning using viral antigens. The advantages of this approach over conventional hybridoma techniques are that approximately 10⁴ times as many antibodies can be produced and screened in a single round, and that new specificities are generated by H and L chain combination which further increases the chance of finding appropriate antibodies.

Other U.S. patents, each incorporated herein by reference, that teach the production of antibodies useful in the present invention include U.S. Pat. No. 5,565,332, which describes the production of chimeric antibodies using a combinatorial approach; U.S. Pat. No. 4,816,567 which describes recombinant immunoglobulin preparations; and U.S. Pat. No. 4,867,973 which describes antibody-therapeutic agent conjugates.

B. Improved Methods of Antibody Production

Antibodies of the present invention can be prepared with an optimized electrofusion method using a PA-4000/PA-101 apparatus with electrode FE-20/1000 fusion chambers (Cyto Pulse Sciences, Inc.). Fusion volume may be 500 μl. Myeloma cells and EBV-transformed human B cells can be washed with RPMI-1640 and Cytofusion medium (Cyto Pulse Sciences, Inc.). Instrument settings are as follows. Pre-fusion dielectrophoresis performed for 15 seconds with an alternating current voltage of 70V at 0.8 Mhz. Cells electroporated with a single square-wave high-voltage direct current pulse lasting 0.04 milliseconds. The pulse frequencies and voltages include a single pulse of 300V or multiple pulses of different decreasing voltages from 280V to 260V. Post-fusion dielectrophoresis accomplished for 30 seconds using an alternating current voltage of 20V at 0.08 Mhz. After fusion, cells are allowed to recover in the fusion electrode for 30 minutes at room temperature, harvested, and then washed once with RPMI-1640 prior to plating in multi-well plates for culture.

After fusion, cells are seeded into 96-well microplates at approximately 6,000 B cells per well (for example 18,000 total cells when a 2:1 myeloma to B cell ratio was used in fusion) in complete RPMI-1640 medium containing 20% heat-inactivated FBS, 2.5 μg/ml amphotericin B, 2 mM L-glutamine, 1 mM sodium pyruvate, 50 μg/ml gentamicin, 60 μg/ml tylosin solution, 100 μM hypoxanthine, 0.4 μM aminopterin, 16 μM thymidine (HAT; Sigma) and 0.5 μM ouabain. After seven days of culture, cells are fed by removing 100 μl culture medium followed by addition of an equal volume of fresh medium containing 100 μM hypoxanthine/16 μM thymidine (HT; Sigma). The number of independent colonies in each well is counted 20 days after fusion. Fusion efficiency on a percentage basis is calculated as the mean number of hybridoma colonies per number of input B cells×100. After initial screening for immunoglobulin (Ig) production by an enzyme-linked immunosorbent assay (ELISA), the hybridoma cells from positive wells are expanded into 24-well plates and cultured in RPMI 1640 containing 20% heat-inactivated FBS, 2 mM glutamine, 1 mM sodium pyruvate and 50 μg/mL gentamicin. Supernatants of the expanded lines are then tested for specificity using an antigen-specific ELISA. The positive hybridoma cells are sub-cloned by serial limiting dilution in 96-well plates at 100, 10, and 0.3 cell-per-well density. The 0.3 cell-per-well limiting dilutions are performed twice to ensure that clones are generated.

To establish optimal drug treatment conditions for selection of human hybridomas, the inventors determined the sensitivity and resistance to drug selection of both transformed B cells and myeloma fusion partner cell lines. Conventional primary B cells die in prolonged culture, but transformed B cells can survive prolonged culture and HAT selection. Human cells are sensitive to ouabain selection, however, therefore the selection of human hybridomas is carried out in the presence of ouabain to eliminate non-fused EBV-transformed B cells. The inventors test the sensitivity of EBV-transformed human B cells to differing concentrations of ouabain and find the minimum concentration for killing EBV-transformed human B cells to be 0.5 μM. More than 99% of EBV-transformed B cells are killed during seven days of culture in medium containing 0.5 μM ouabain. The inventors then test the resistance of seven myeloma fusion partner cell lines to 0.5 μM ouabain.

Synthetic oligodeoxynucleotides (ODNs) that contain immunostimulatory CpG motifs trigger an immunomodulatory cascade that involves B and T cells, natural killer cells and professional antigen-presenting cells. The inventors propose adding CpG ODNs to the EBV transformation medium for human B cells. In order to enrich for the percentage of antigen-specific B cell numbers in pre-fusion B cell samples, one may transfer smaller numbers of human B cells in multiple wells of 384-well plates using CpG and EBV.

C. Antibodies of the Present Invention

Antibodies according to the present invention may be defined, in the first instance, by their binding specificity. Those of skill in the art, by assessing the binding affinity of a given antibody using techniques well known to those of skill in the art, can determine whether such antibodies fall within the scope of the instant claims.

In one aspect, there is provided a monoclonal antibody that binds to globular head region of influenza virus hemagglutinin. Another type of antibody that bindings to the stalk region of the virus, including epitopes that are found exclusively in the long alpha helical repeat region, and those that are found in other locations within the stalk.

Antibodies may also be defined based on Group or strain specificity, or on the ability to cross-react with Group 1 viruses or Group 2 viruses, or even to cross-react between Group 1 and 2 viruses. Yet another way of categorizing the antibodies of the present invention is by their activity. This could included the ability to neutralize virus in the absence of complement, to inhibit hemagglutination, or to do both. Finally, the antibody may be defined in particular by reference to heavy/light chain variable region sequences.

As provided in the Table A, various combinations of these properties are set forth antibody clone designations and sequences are set forth. Table B provides clone designation-sequence correlations.

TABLE A
INFLUENZA HUMAN MABS

| Domain | Class of antibody | HAI | Clone examples |
|---|---|---|---|
| Head | Binds all HAs, in the head domain | 3E22 = ~490 ng/ml | 3E22, 5I17, 5G11 |
| | Group 1 specific head | | |
| | Pan H1 subtype head | | 8F24, 12F14, 13H6, 5J8, |
| | | | 1A17, 2K1E 2O10, 4A10, |
| | | | 4K8, 6D9 |
| | Early 20^{th} century H1 head (for ex, | yes | 1F1, 1I20, 2D1, 4D20, |
| | 1918, 1930) | | 2B12 |
| | Late 20^{th} century H1 head (for ex | | 3J10, 11I12, 12D7, 5I5, |
| | 1999 seasonal HA) | | 12I1, 2C7 |
| | 2009 pandemic H1 specific | | |
| | 1957 H2 specific | | 17L8, 18E6, 8G6, 25F7, |
| | | | 8F8, 8M2, 2G1, 2H22, |
| | | | 8K20, 4I4, 4E2 |
| | 1968 H3 specific | | 7A13, 2L15, 11J19, 15C13 |
| | Pan H5 head domain | yes | 13H19, 4K4, |
| | H5 Vietnam-specific head domain | yes | 18D16, 2D16, 2M13, 6N1 |
| LAH | LAH Group 1 + 2 | | 6K14, 6J24, 5K24, 5F20 |
| | LAH Group 1 specific | | 3N6, 5F18, 13H19, 1O19, |
| | | | 5O20, 1B14, 3D19, 3D3, |
| | | | 6I19, 1K12, 2F18, 17F16 |
| | LAH Group 2 specific | | 5J4, 1C23 |
| Non-LAH | Conventional stem epitope, Ab gene | | 8D4, 10A14 |
| | V_{H}1-69 | | |
| | Conventional stem epitope, non-gene | | 10E15 |
| | V_{H}1-69 | | |

TABLE B
CLONE DESIGNATION/SEQUENCE CORRELATION

| | | | Light | | |
|---|---|---|---|---|---|
| Clone | Heavy Chain | Heavy Chain | Chain | Light Chain | Neutralizing |
| Design. | AA | NA | AA | NA | Activity |
| 8F24 | 1 | 2 | 3 | 4 | + |
| 3E22 | 5 | 6 | 7 | 8 | − |
| 5I17 | 9 | 10 | 11 | 12 | − |
| 3N6 | 13 | 14 | 15 | 16 | − |
| 10E15 | 17 | 18 | 19 | 20 | − |
| 10A14 | 21 | 22 | 23 | 24 | + |
| 8D4 | 25 | 26 | 27 | 28 | + |
| 6J24 | 29 | 30 | 31 | 32 | − |
| 6K14 | 33 | 34 | 35 | 36 | − |
| 25J3 | 37 | 38 | 39 | 40 | − |
| 25F7 | 41 | 42 | 43 | 44 | + |
| 8F8 | 45 | 46 | 47 | 48 | + |
| 18G6 | 49 | 50 | 51 | 52 | − |
| 7A13 | 53 | 54 | 55 | 56 | + |
| 3J10 | 57 | 58 | 59 | 60 | − |
| 8M2 | 65 | 66 | 67 | 68 | + |
| 2G1 | 69 | 70 | 71 | 72 | + |
| 11J19 | 73 | 74 | 75 | 76 | + |
| 5J8 | 77 | 78 | 79 | 80 | + |
| 5B6 | 81 | 82 | 83 | 84 | + |
| 2K11 | 85 | 86 | 87 | 88 | + |
| 2O10 | 89 | 90 | 91 | 92 | + |
| 4A10 | 93 | 94 | 95 | 96 | + |
| 4K8 | 97 | 98 | 99 | 100 | + |
| 6D9 | 101 | 102 | 103 | 104 | + |

D. Engineering of Antibody Sequences

In various embodiments, one may choose to engineer sequences of the identified antibodies for a variety of reasons, such as improved expression, improved cross-reactivity or diminished off-target binding. The following is a general discussion of relevant techniques for antibody engineering.

Hybridomas may cultured, then cells lysed, and total RNA extracted. Random hexamers may be used with RT to generate cDNA copies of RNA, and then PCR performed using a multiplex mixture of PCR primers expected to amplify all human variable gene sequences. PCR product can be cloned into pGEM-T Easy vector, then sequenced by automated DNA sequencing using standard vector primers. Assay of binding and neutralization may be performed using antibodies collected from hybridoma supernatants and purified by FPLC, using Protein G columns.

Recombinant full length IgG antibodies were generated by subcloning heavy and light chain Fv DNAs from the cloning vector into a Lonza pConIgG1 or pConK2 plasmid vector, transfected into 293 Freestyle cells or Lonza CHO cells, and antibodies were collected an purified from the CHO cell supernatant.

The rapid availability of antibody produced in the same host cell and cell culture process as the final cGMP manufacturing process has the potential to reduce the duration of process development programs. Lonza has developed a generic method using pooled transfectants grown in CDACF medium, for the rapid production of small quantities (up to 50 g) of antibodies in CHO cells. Although slightly slower than a true transient system, the advantages include a higher product concentration and use of the same host and process as the production cell line. Example of growth and productivity of GS-CHO pools, expressing a model antibody, in a disposable bioreactor: in a disposable bag bioreactor culture (5 L working volume) operated in fed-batch mode, a harvest antibody concentration of 2 g/L was achieved within 9 weeks of transfection.

pCon Vectors™ are an easy way to re-express whole antibodies. The constant region vectors are a set of vectors offering a range of immunoglobulin constant region vectors cloned into the pEE vectors. These vectors offer easy construction of full length antibodies with human constant regions and the convenience of the GS System™.

Antibody molecules will comprise fragments (such as F(ab′), F(ab′)₂) that are produced, for example, by the proteolytic cleavage of the mAbs, or single-chain immunoglobulins producible, for example, via recombinant means. Such antibody derivatives are monovalent. In one embodiment, such fragments can be combined with one another, or with other antibody fragments or receptor ligands to form “chimeric” binding molecules. Significantly, such chimeric molecules may contain substituents capable of binding to different epitopes of the same molecule.

It may be desirable to “humanize” antibodies produced in non-human hosts in order to attenuate any immune reaction when used in human therapy. Such humanized antibodies may be studied in an in vitro or an in vivo context. Humanized antibodies may be produced, for example by replacing an immunogenic portion of an antibody with a corresponding, but non-immunogenic portion (i.e., chimeric antibodies). PCT Application PCT/US86/02269; EP Application 184,187; EP Application 171,496; EP Application 173,494; PCT Application WO 86/01533; EP Application 125,023; Sun et al. (1987); Wood et al. (1985); and Shaw et al. (1988); all of which references are incorporated herein by reference. General reviews of “humanized” chimeric antibodies are provided by Morrison (1985); also incorporated herein by reference. “Humanized” antibodies can alternatively be produced by CDR or CEA substitution. Jones et al. (1986); Verhoeyen et al. (1988); Beidler et al. (1988); all of which are incorporated herein by reference.

In related embodiments, the antibody is a derivative of the disclosed antibodies, e.g., an antibody comprising the CDR sequences identical to those in the disclosed antibodies (e.g., a chimeric, humanized or CDR-grafted antibody). In yet a further embodiment, the antibody is a fully human recombinant antibody.

Alternatively, one may wish to make modifications, such as introducing conservative changes into an antibody molecule. In making such changes, the hydropathic index of amino acids may be considered. The importance of the hydropathic amino acid index in conferring interactive biologic function on a protein is generally understood in the art (Kyte and Doolittle, 1982). It is accepted that the relative hydropathic character of the amino acid contributes to the secondary structure of the resultant protein, which in turn defines the interaction of the protein with other molecules, for example, enzymes, substrates, receptors, DNA, antibodies, antigens, and the like.

It also is understood in the art that the substitution of like amino acids can be made effectively on the basis of hydrophilicity. U.S. Pat. No. 4,554,101, incorporated herein by reference, states that the greatest local average hydrophilicity of a protein, as governed by the hydrophilicity of its adjacent amino acids, correlates with a biological property of the protein. As detailed in U.S. Pat. No. 4,554,101, the following hydrophilicity values have been assigned to amino acid residues: basic amino acids: arginine (+3.0), lysine (+3.0), and histidine (−0.5); acidic amino acids: aspartate (+3.0±1), glutamate (+3.0±1), asparagine (+0.2), and glutamine (+0.2); hydrophilic, nonionic amino acids: serine (+0.3), asparagine (+0.2), glutamine (+0.2), and threonine (−0.4), sulfur containing amino acids: cysteine (−1.0) and methionine (−1.3); hydrophobic, nonaromatic amino acids: valine (−1.5), leucine (−1.8), isoleucine (−1.8), proline (−0.5±1), alanine (−0.5), and glycine (0); hydrophobic, aromatic amino acids: tryptophan (−3.4), phenylalanine (−2.5), and tyrosine (−2.3).

It is understood that an amino acid can be substituted for another having a similar hydrophilicity and produce a biologically or immunologically modified protein. In such changes, the substitution of amino acids whose hydrophilicity values are within ±2 is preferred, those that are within ±1 are particularly preferred, and those within ±0.5 are even more particularly preferred.

As outlined above, amino acid substitutions generally are based on the relative similarity of the amino acid side-chain substituents, for example, their hydrophobicity, hydrophilicity, charge, size, and the like. Exemplary substitutions that take into consideration the various foregoing characteristics are well known to those of skill in the art and include: arginine and lysine; glutamate and aspartate; serine and threonine; glutamine and asparagine; and valine, leucine and isoleucine.

The present invention also contemplates isotype modification. By modifying the Fe region to have a different isotype, different functionalities can be achieved. For example, changing to IgG₁ can increase antibody dependent cell cytotoxicity, switching to class A can improve tissue distribution, and switching to class M can improve valency.

Modified antibodies may be made by any technique known to those of skill in the art, including expression through standard molecular biological techniques, or the chemical synthesis of polypeptides. Methods for recombinant expression are addressed elsewhere in this document.

E. Single Chain Antibodies

A Single Chain Variable Fragment (scFv) is a fusion of the variable regions of the heavy and light chains of immunoglobulins, linked together with a short (usually serine, glycine) linker. This chimeric molecule retains the specificity of the original immunoglobulin, despite removal of the constant regions and the introduction of a linker peptide. This modification usually leaves the specificity unaltered. These molecules were created historically to facilitate phage display where it is highly convenient to express the antigen binding domain as a single peptide. Alternatively, scFv can be created directly from subcloned heavy and light chains derived from a hybridoma. Single chain variable fragments lack the constant Fc region found in complete antibody molecules, and thus, the common binding sites (e.g., protein A/G) used to purify antibodies. These fragments can often be purified/immobilized using Protein L since Protein L interacts with the variable region of kappa light chains.

Flexible linkers generally are comprised of helix- and turn-promoting amino acid residues such as alaine, serine and glycine. However, other residues can function as well. Tang et al. (1996) used phage display as a means of rapidly selecting tailored linkers for single-chain antibodies (scFvs) from protein linker libraries. A random linker library was constructed in which the genes for the heavy and light chain variable domains were linked by a segment encoding an 18-amino acid polypeptide of variable composition. The scFv repertoire (approx. 5×10⁶ different members) was displayed on filamentous phage and subjected to affinity selection with hapten. The population of selected variants exhibited significant increases in binding activity but retained considerable sequence diversity. Screening 1054 individual variants subsequently yielded a catalytically active scFv that was produced efficiently in soluble form. Sequence analysis revealed a conserved proline in the linker two residues after the V_{H} C terminus and an abundance of arginines and prolines at other positions as the only common features of the selected tethers.

The recombinant antibodies of the present invention may also involve sequences or moieties that permit dimerization or multimerization of the receptors. Such sequences include those derived from IgA, which permit formation of multimers in conjunction with the J-chain. Another multimerization domain is the Gal4 dimerization domain. In other embodiments, the chains may be modified with agents such as biotin/avidin, which permit the combination of two antibodies.

In a separate embodiment, a single-chain antibody can be created by joining receptor light and heavy chains using a non-peptide linker or chemical unit. Generally, the light and heavy chains will be produced in distinct cells, purified, and subsequently linked together in an appropriate fashion (i.e., the N-terminus of the heavy chain being attached to the C-terminus of the light chain via an appropriate chemical bridge).

Cross-linking reagents are used to form molecular bridges that tie functional groups of two different molecules, e.g., a stabilizing and coagulating agent. However, it is contemplated that dimers or multimers of the same analog or heteromeric complexes comprised of different analogs can be created. To link two different compounds in a step-wise manner, hetero-bifunctional cross-linkers can be used that eliminate unwanted homopolymer formation.

An exemplary hetero-bifunctional cross-linker contains two reactive groups: one reacting with primary amine group (e.g., N-hydroxy succinimide) and the other reacting with a thiol group (e.g., pyridyl disulfide, maleimides, halogens, etc.). Through the primary amine reactive group, the cross-linker may react with the lysine residue(s) of one protein (e.g., the selected antibody or fragment) and through the thiol reactive group, the cross-linker, already tied up to the first protein, reacts with the cysteine residue (free sulfhydryl group) of the other protein (e.g., the selective agent).

It is preferred that a cross-linker having reasonable stability in blood will be employed. Numerous types of disulfide-bond containing linkers are known that can be successfully employed to conjugate targeting and therapeutic/preventative agents. Linkers that contain a disulfide bond that is sterically hindered may prove to give greater stability in vivo, preventing release of the targeting peptide prior to reaching the site of action. These linkers are thus one group of linking agents.

Another cross-linking reagent is SMPT, which is a bifunctional cross-linker containing a disulfide bond that is “sterically hindered” by an adjacent benzene ring and methyl groups. It is believed that steric hindrance of the disulfide bond serves a function of protecting the bond from attack by thiolate anions such as glutathione which can be present in tissues and blood, and thereby help in preventing decoupling of the conjugate prior to the delivery of the attached agent to the target site.

The SMPT cross-linking reagent, as with many other known cross-linking reagents, lends the ability to cross-link functional groups such as the SH of cysteine or primary amines (e.g., the epsilon amino group of lysine). Another possible type of cross-linker includes the hetero-bifunctional photoreactive phenylazides containing a cleavable disulfide bond such as sulfosuccinimidyl-2-(p-azido salicylamido) ethyl-1,3′-dithiopropionate. The N-hydroxy-succinimidyl group reacts with primary amino groups and the phenylazide (upon photolysis) reacts non-selectively with any amino acid residue.

In addition to hindered cross-linkers, non-hindered linkers also can be employed in accordance herewith. Other useful cross-linkers, not considered to contain or generate a protected disulfide, include SATA, SPDP and 2-iminothiolane (Wawrzynczak & Thorpe, 1987). The use of such cross-linkers is well understood in the art. Another embodiment involves the use of flexible linkers.

U.S. Pat. No. 4,680,338, describes bifunctional linkers useful for producing conjugates of ligands with amine-containing polymers and/or proteins, especially for forming antibody conjugates with chelators, drugs, enzymes, detectable labels and the like. U.S. Pat. Nos. 5,141,648 and 5,563,250 disclose cleavable conjugates containing a labile bond that is cleavable under a variety of mild conditions. This linker is particularly useful in that the agent of interest may be bonded directly to the linker, with cleavage resulting in release of the active agent. Particular uses include adding a free amino or free sulfhydryl group to a protein, such as an antibody, or a drug.

U.S. Pat. No. 5,856,456 provides peptide linkers for use in connecting polypeptide constituents to make fusion proteins, e.g., single chain antibodies. The linker is up to about 50 amino acids in length, contains at least one occurrence of a charged amino acid (preferably arginine or lysine) followed by a proline, and is characterized by greater stability and reduced aggregation. U.S. Pat. No. 5,880,270 discloses aminooxy-containing linkers useful in a variety of immunodiagnostic and separative techniques.

F. Purification

In certain embodiments, the antibodies of the present invention may be purified. The term “purified,” as used herein, is intended to refer to a composition, isolatable from other components, wherein the protein is purified to any degree relative to its naturally-obtainable state. A purified protein therefore also refers to a protein, free from the environment in which it may naturally occur. Where the term “substantially purified” is used, this designation will refer to a composition in which the protein or peptide forms the major component of the composition, such as constituting about 50%, about 60%, about 70%, about 80%, about 90%, about 95% or more of the proteins in the composition.

Protein purification techniques are well known to those of skill in the art. These techniques involve, at one level, the crude fractionation of the cellular milieu to polypeptide and non-polypeptide fractions. Having separated the polypeptide from other proteins, the polypeptide of interest may be further purified using chromatographic and electrophoretic techniques to achieve partial or complete purification (or purification to homogeneity). Analytical methods particularly suited to the preparation of a pure peptide are ion-exchange chromatography, exclusion chromatography; polyacrylamide gel electrophoresis; isoelectric focusing. Other methods for protein purification include, precipitation with ammonium sulfate, PEG, antibodies and the like or by heat denaturation, followed by centrifugation; gel filtration, reverse phase, hydroxylapatite and affinity chromatography; and combinations of such and other techniques.

In purifying an antibody of the present invention, it may be desirable to express the polypeptide in a prokaryotic or eukaryotic expression system and extract the protein using denaturing conditions. The polypeptide may be purified from other cellular components using an affinity column, which binds to a tagged portion of the polypeptide. As is generally known in the art, it is believed that the order of conducting the various purification steps may be changed, or that certain steps may be omitted, and still result in a suitable method for the preparation of a substantially purified protein or peptide.

Commonly, complete antibodies are fractionated utilizing agents (i.e., protein A) that bind the Fc portion of the antibody. Alternatively, antigens my be used to simultaneously purify and select appropriate antibodies. Such methods often utilize the selection agent bound to a support, such as a column, filter or bead. The antibodies is bound to a support, contaminants removed (e.g., washed away), and the antibodies released by applying conditions (salt, heat, etc.).

Various methods for quantifying the degree of purification of the protein or peptide will be known to those of skill in the art in light of the present disclosure. These include, for example, determining the specific activity of an active fraction, or assessing the amount of polypeptides within a fraction by SDS/PAGE analysis. Another method for assessing the purity of a fraction is to calculate the specific activity of the fraction, to compare it to the specific activity of the initial extract, and to thus calculate the degree of purity. The actual units used to represent the amount of activity will, of course, be dependent upon the particular assay technique chosen to follow the purification and whether or not the expressed protein or peptide exhibits a detectable activity.

It is known that the migration of a polypeptide can vary, sometimes significantly, with different conditions of SDS/PAGE (Capaldi et al., 1977). It will therefore be appreciated that under differing electrophoresis conditions, the apparent molecular weights of purified or partially purified expression products may vary.

### IV. ACTIVE/PASSIVE IMMUNIZATION AND TREATMENT OF INFLUENZA INFECTION

A. Formulation and Administration

The present invention provides pharmaceutical compositions comprising anti-influenza virus antibodies and antigens for generating the same. Such compositions comprise a prophylactically or therapeutically effective amount of an antibody or a fragment thereof, or a peptide immunogen, and a pharmaceutically acceptable carrier. In a specific embodiment, the term “pharmaceutically acceptable” means approved by a regulatory agency of the Federal or a state government or listed in the U.S. Pharmacopeia or other generally recognized pharmacopeia for use in animals, and more particularly in humans. The term “carrier” refers to a diluent, excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Water is a particular carrier when the pharmaceutical composition is administered intravenously. Saline solutions and aqueous dextrose and glycerol solutions can also be employed as liquid carriers, particularly for injectable solutions. Other suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like.

The composition, if desired, can also contain minor amounts of wetting or emulsifying agents, or pH buffering agents. These compositions can take the form of solutions, suspensions, emulsion, tablets, pills, capsules, powders, sustained-release formulations and the like. Oral formulations can include standard carriers such as pharmaceutical grades of mannitol, lactose, starch, magnesium stearate, sodium saccharine, cellulose, magnesium carbonate, etc. Examples of suitable pharmaceutical agents are described in “Remington's Pharmaceutical Sciences.” Such compositions will contain a prophylactically or therapeutically effective amount of the antibody or fragment thereof, preferably in purified form, together with a suitable amount of carrier so as to provide the form for proper administration to the patient. The formulation should suit the mode of administration, which can be oral, intravenous, intraarterial, intrabuccal, intranasal, nebulized, bronchial inhalation, or delivered by mechanical ventilation.

Active vaccines of the present invention, as described herein, can be formulated for parenteral administration, e.g., formulated for injection via the intradermal, intravenous, intramuscular, subcutaneous, or even intraperitoneal routes. Administration by the intradermal and intramuscular routes are specifically contemplated. The vaccine could alternatively be administered by a topical route directly to the mucosa, for example by nasal drops, inhalation, or by nebulizer. Pharmaceutically acceptable salts, include the acid salts and those which are formed with inorganic acids such as, for example, hydrochloric or phosphoric acids, or such organic acids as acetic, oxalic, tartaric, mandelic, and the like. Salts formed with the free carboxyl groups may also be derived from inorganic bases such as, for example, sodium, potassium, ammonium, calcium, or ferric hydroxides, and such organic bases as isopropylamine, trimethylamine, 2-ethylamino ethanol, histidine, procaine, and the like.

Passive transfer of antibodies, known as artificially acquired passive immunity, generally will involve the use of intravenous or intramuscular injections. The forms of antibody can be human or animal blood plasma or serum, as pooled human immunoglobulin for intravenous (IVIG) or intramuscular (IG) use, as high-titer human IVIG or IG from immunized or from donors recovering from disease, and as monoclonal antibodies (MAb). Such immunity generally lasts for only a short period of time, and there is also a potential risk for hypersensitivity reactions, and serum sickness, especially from gamma globulin of non-human origin. However, passive immunity provides immediate protection. The antibodies will be formulated in a carrier suitable for injection, i.e., sterile and syringeable.

Generally, the ingredients of compositions of the invention are supplied either separately or mixed together in unit dosage form, for example, as a dry lyophilized powder or water-free concentrate in a hermetically sealed container such as an ampoule or sachette indicating the quantity of active agent. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

The compositions of the invention can be formulated as neutral or salt forms. Pharmaceutically acceptable salts include those formed with anions such as those derived from hydrochloric, phosphoric, acetic, oxalic, tartaric acids, etc., and those formed with cations such as those derived from sodium, potassium, ammonium, calcium, ferric hydroxides, isopropylamine, triethylamine, 2-ethylamino ethanol, histidine, procaine, etc.

B. Combination Therapy

In order to increase the effectiveness of the antibody therapy of the present invention, it may be desirable to combine this treatment with other agents effective at treating or preventing influenza virus infections, e.g., oseltamivir (Tamiflu™) and zanamivir (Relenza™). This process may involve administering to the patient the antibody of the present invention the other agent(s) at the same time. This may be achieved by use of a single pharmaceutical composition that includes both agents, or by administering two distinct compositions at the same time, wherein one composition includes the antibody of the present invention and the other includes the second agent(s).

The two therapies may be given in either order and may precede or follow the other treatment by intervals ranging from minutes to weeks. In embodiments where the other agents are applied separately, one would generally ensure that a significant period of time did not expire between the time of each delivery, such that the agents would still be able to exert an advantageously combined effect on the patient. In such instances, it is contemplated that one may administer both modalities within about 12-24 h of each other and, more preferably, within about 6-12 h of each other. In some situations, it may be desirable to extend the time period for treatment significantly, however, where several d (2, 3, 4, 5, 6 or 7) to several wk (1, 2, 3, 4, 5, 6, 7 or 8) lapse between the respective administrations.

Various combinations may be employed, the immunogen or antibody treatment of the present invention is “A” and the secondary treatment is “B”:
A/B/A B/A/B B/B/A A/A/B A/B/B B/A/A A/B/B/B B/A/B/B
B/B/B/A B/B/A/B A/A/B/B A/B/A/B A/B/B/A B/B/A/A
B/A/B/A B/A/A/B A/A/A/B B/A/A/A A/B/A/A A/A/B/A
   Administration of the secondary agent will follow general protocols for that drug, taking into account the toxicity, if any. It is expected that the treatment cycles would be repeated as necessary.

i. Oseltamivir

Oseltamivir is an antiviral drug that is used in the treatment and prophylaxis of both Influenzavirus A and Influenzavirus B infection. Like zanamivir, oseltamivir is a neuraminidase inhibitor. It acts as a transition-state analogue inhibitor of influenza neuraminidase, preventing progeny virions from emerging from infected cells.

Oseltamivir was the first orally active neuraminidase inhibitor commercially developed. It is a prodrug, which is hydrolysed hepatically to the active metabolite, the free carboxylate of oseltamivir (GS4071). It was developed by U.S.-based Gilead Sciences and is currently marketed by Hoffmann-La Roche (Roche) under the trade name Tamiflu®. In Japan, it is marketed by Chugai Pharmaceutical Co., which is more than 50% owned by Roche. Oseltamivir is generally available by prescription only.

Roche estimates that 50 million people have been treated with oseltamivir. The majority of these have been in Japan, where an estimated 35 million have been treated.

In 2009, with increasing fears about the potential for a new influenza pandemic, oseltamivir has received substantial media attention. Governments, corporations, and even some private individuals are stockpiling the drug (which is discouraged by government bodies). Production is currently sufficient to meet the demand for seasonal influenza and for government stockpiling. It is possible that shortages could recur in the event of an actual influenza pandemic.

Oseltamivir is indicated for the treatment and prevention of infections due to influenza A and B virus in people at least one year of age. The usual adult dosage for treatment of influenza is 75 mg twice daily for 5 days, beginning within 2 days of the appearance of symptoms and with decreased doses for children and patients with renal impairment. Oseltamivir may be given as a preventive measure either during a community outbreak or following close contact with an infected individual. Standard prophylactic dosage is 75 mg once daily for patients aged 13 and older, which has been shown to be safe and effective for up to six weeks. The importance of early treatment is that the NA protein inhibition is more effective within the first 48 hours. If the virus has replicated and infected many cells the effectiveness of this medication will be severely diminished, especially over time.

It has been suggested that co-administration of oseltamivir with probenecid could extend a limited supply of oseltamivir. Probenecid reduces renal excretion of the active metabolite of oseltamivir. One study showed that 500 mg of probenecid given every six hours doubled both the peak plasma concentration (Cₘₐₓ) and the half-life of oseltamivir, increasing overall systemic exposure (AUC) by 150 percent. Although the evidence for this interaction comes from a study by Roche, it was publicised only in October 2005 by a doctor who had reviewed the data. Probenecid was used in similar fashion during World War II to extend limited supplies of penicillin. It is still used to increase penicillin concentrations in serious infections.

Oseltamivir is prescribed as capsules (containing oseltamivir phosphate 98.5 mg equivalent to oseltamivir 75 mg) and as a powder for oral suspension (oseltamivir phosphate equivalent to oseltamivir 12 mg/mL).

As with other antivirals, resistance to the agent was expected with widespread use of oseltamivir, though the emergence of resistant viruses was expected to be less frequent than with amantadine or rimantadine. The resistance rate reported during clinical trials up to July 2004 was 0.33% in adults, 4.0% in children, and 1.26% overall. Mutations conferring resistance are single amino acid residue substitutions in the neuraminidase enzyme.

Mutant H3N2 influenza A virus isolates resistant to oseltamivir were found in 18% of a group of 50 Japanese children treated with oseltamivir. This rate was similar to another study where resistant isolates of H1N1 influenza virus were found in 16.3% of another cohort of Japanese children. Several explanations were proposed by the authors of the studies for the higher-than-expected resistance rate detected. First, children typically have a longer infection period, giving a longer time for resistance to develop. Second, the more recent study is purported to have used more rigorous detection techniques than previous studies.

High-level resistance has been detected in one girl suffering from H5N1 avian influenza in Vietnam. She was being treated with oseltamivir at time of detection. Others have described resistance development in two more Vietnamese patients suffering from H5N1, and compare their cases with six others. They suggest that the emergence of a resistant strain may be associated with a patient's clinical deterioration. They also note that the recommended dosage of oseltamivir does not always completely suppress viral replication, a situation that could favor the emergence of resistant strains.

The genetic sequence for the neuraminidase enzyme is highly conserved across virus strains. This means that there are relatively few variations, and there is also evidence that variations that do occur tend to be less “fit.” Thus, mutations that convey resistance to oseltamivir may also tend to cripple the virus by giving it an otherwise less-functional enzyme. The lack of variation in neuraminidase gives two advantages to oseltamivir and zanamivir, the drugs that target that enzyme. First, these drugs work on a broader spectrum of influenza strains. Second, the development of a robust, resistant virus strain appears to be less likely.

ii. Zanamivir

Zanamivir is a neuraminidase inhibitor used in the treatment of and prophylaxis of both Influenzavirus A and Influenzavirus B. Zanamivir was the first neuraminidase inhibitor commercially developed. It is currently marketed by GlaxoSmithKline under the trade name Relenza®, and was developed by a team of scientists at the Victorian College of Pharmacy at Monash University in Melbourne, Australia.

The approval of Relenza® in the United States was controversial. In 1999 a Food and Drug Administration (FDA) advisory committee voted 13 to 4 not to approve the drug because of limited data on efficacy and safety concerns. The drug was approved later in 1999.

Relenza® is a part of a range of neuraminidase inhibitor medications. This medication was designed to attack the infected host cells, preventing the virus from spreading throughout other cells in the body and thus reducing the amount of time the virus can survive.

In 1990 licensing of zanamivir was sold to Glaxo, which is now known as GlaxoSmithKline (GSK). In 1999, the product was approved for marketing in the US and subsequently has been registered by GSK in a total of 70 countries. (GlaxoSmithKline News release, 2006) Tamiflu®, Relenza®'s main competitor, was proven in 2006 to not be as effective at treating influenza viruses as Relenza®. As a result in August 2006 Germany announced that it would buy 1.7 million doses of Relenza® as part of its preparation strategy against bird flu.

Zanamivir proved to be a potent and effective inhibitor of influenza neuraminidase. It works by binding to the active site of the neuraminidase protein, rendering the influenza virus unable to escape its host cell and infect others. It is also an inhibitor of influenza virus replication in vitro and in vivo; however this did not necessarily translate into a successful clinical treatment for influenza. In clinical trials it was found that zanamivir was able to reduce the time to symptom resolution by 1.5 days if therapy was started within 48 hours of the onset of symptoms.

Relenza® is a safe and effective treatment for influenza, but must be administered soon after the first symptoms appear. Six to 12 hours is ideal. In most countries the drugs can only be obtained with a doctor's prescription, and usually the time taken to get a prescription renders them ineffective.

A further limitation is the poor oral bioavailability of zanamivir. This meant that oral dosing was impossible, limiting dosing to the parenteral (that is, intravenous) routes. This restricted its usage when treating the elderly because it may induce bronchospasm. Zanamivir, therefore, is administered by inhalation—a route that was chosen for patient compliance with therapy. But this route of administration is not acceptable to many in the community.

Zanamivir is specific to the influenza virus, has not been known to cause toxic effects, and does not spread around through the body's systemic circulation. It also shows no signs of viral resistance. However, due to a lack of reports or evidence about its toxicity, the FDA does not license it for use in children under 7 years of age.

Relenza® is at least as effective as Tamiflu® and has fewer side effects, including nausea and headaches, according to one report. The report, based on data compiled from the companies' clinical trials and from subsequent studies, also says there is no evidence of resistance to Relenza®, compared with resistance levels of up to 18% in those taking Tamiflu®.

iii. Amantadine and Rimantadine

The antiviral drugs amantadine and rimantadine are designed to block a viral ion channel (M2 protein) and prevent the virus from infecting cells. These drugs are sometimes effective against influenza A if given early in the infection but are always ineffective against influenza B. Measured resistance to amantadine and rimantadine in American isolates of H3N2 has increased to 91% in 2005. In contrast to neuraminidase inhibitors, amantadine and rimantadine have not proven effective against the 2009 “swine” flu.

### V. ANTIBODY CONJUGATES

Antibodies of the present invention may be linked to at least one agent to form an antibody conjugate. In order to increase the efficacy of antibody molecules as diagnostic or therapeutic agents, it is conventional to link or covalently bind or complex at least one desired molecule or moiety. Such a molecule or moiety may be, but is not limited to, at least one effector or reporter molecule. Effector molecules comprise molecules having a desired activity, e.g., cytotoxic activity. Non-limiting examples of effector molecules which have been attached to antibodies include toxins, anti-tumor agents, therapeutic enzymes, radionuclides, antiviral agents, chelating agents, cytokines, growth factors, and oligo- or polynucleotides. By contrast, a reporter molecule is defined as any moiety which may be detected using an assay. Non-limiting examples of reporter molecules which have been conjugated to antibodies include enzymes, radiolabels, haptens, fluorescent labels, phosphorescent molecules, chemiluminescent molecules, chromophores, photoaffinity molecules, colored particles or ligands, such as biotin.

Antibody conjugates are generally preferred for use as diagnostic agents. Antibody diagnostics generally fall within two classes, those for use in in vitro diagnostics, such as in a variety of immunoassays, and those for use in vivo diagnostic protocols, generally known as “antibody-directed imaging.” Many appropriate imaging agents are known in the art, as are methods for their attachment to antibodies (see, for e.g., U.S. Pat. Nos. 5,021,236, 4,938,948, and 4,472,509). The imaging moieties used can be paramagnetic ions, radioactive isotopes, fluorochromes, NMR-detectable substances, and X-ray imaging agents.

In the case of paramagnetic ions, one might mention by way of example ions such as chromium (III), manganese (II), iron (III), iron (II), cobalt (II), nickel (II), copper (II), neodymium (III), samarium (III), ytterbium (III), gadolinium (III), vanadium (II), terbium (III), dysprosium (III), holmium (III) and/or erbium (III), with gadolinium being particularly preferred. Ions useful in other contexts, such as X-ray imaging, include but are not limited to lanthanum (III), gold (III), lead (II), and especially bismuth (III).

In the case of radioactive isotopes for therapeutic and/or diagnostic application, one might mention astatine²¹¹, ¹⁴carbon, ⁵¹chromium, ³⁶chlorine, ⁵⁷cobalt, ⁵⁸cobalt, copper⁶⁷, ¹⁵²Eu, gallium⁶⁷, ³hydrogen, iodine¹²³, iodine¹²⁵, iodine¹³¹, indium¹¹¹, ⁵⁹iron, ³²phosphorus, rhenium¹⁸⁶, rhenium¹⁸⁸, ⁷⁵selenium, ³⁵sulphur, technicium^{99m} and/or yttrium⁹⁰. ¹²⁵I is often being preferred for use in certain embodiments, and technicium^{99m} and/or indium¹¹¹ are also often preferred due to their low energy and suitability for long range detection. Radioactively labeled monoclonal antibodies of the present invention may be produced according to well-known methods in the art. For instance, monoclonal antibodies can be iodinated by contact with sodium and/or potassium iodide and a chemical oxidizing agent such as sodium hypochlorite, or an enzymatic oxidizing agent, such as lactoperoxidase. Monoclonal antibodies according to the invention may be labeled with technetium^{99m} by ligand exchange process, for example, by reducing pertechnate with stannous solution, chelating the reduced technetium onto a Sephadex column and applying the antibody to this column. Alternatively, direct labeling techniques may be used, e.g., by incubating pertechnate, a reducing agent such as SNCl₂, a buffer solution such as sodium-potassium phthalate solution, and the antibody. Intermediary functional groups which are often used to bind radioisotopes which exist as metallic ions to antibody are diethylenetriaminepentaacetic acid (DTPA) or ethylene diaminetetracetic acid (EDTA).

Among the fluorescent labels contemplated for use as conjugates include Alexa 350, Alexa 430, AMCA, BODIPY 630/650, BODIPY 650/665, BODIPY-FL, BODIPY-R6G, BODIPY-TMR, BODIPY-TRX, Cascade Blue, Cy3, Cy5,6-FAM, Fluorescein Isothiocyanate, HEX, 6-JOE, Oregon Green 488, Oregon Green 500, Oregon Green 514, Pacific Blue, REG, Rhodamine Green, Rhodamine Red, Renographin, ROX, TAMRA, TET, Tetramethylrhodamine, and/or Texas Red.

Another type of antibody conjugates contemplated in the present invention are those intended primarily for use in vitro, where the antibody is linked to a secondary binding ligand and/or to an enzyme (an enzyme tag) that will generate a colored product upon contact with a chromogenic substrate. Examples of suitable enzymes include urease, alkaline phosphatase, (horseradish) hydrogen peroxidase or glucose oxidase. Preferred secondary binding ligands are biotin and avidin and streptavidin compounds. The use of such labels is well known to those of skill in the art and are described, for example, in U.S. Pat. Nos. 3,817,837, 3,850,752, 3,939,350, 3,996,345, 4,277,437, 4,275,149 and 4,366,241.

Yet another known method of site-specific attachment of molecules to antibodies comprises the reaction of antibodies with hapten-based affinity labels. Essentially, hapten-based affinity labels react with amino acids in the antigen binding site, thereby destroying this site and blocking specific antigen reaction. However, this may not be advantageous since it results in loss of antigen binding by the antibody conjugate.

Molecules containing azido groups may also be used to form covalent bonds to proteins through reactive nitrene intermediates that are generated by low intensity ultraviolet light (Potter and Haley, 1983). In particular, 2- and 8-azido analogues of purine nucleotides have been used as site-directed photoprobes to identify nucleotide binding proteins in crude cell extracts (Owens & Haley, 1987; Atherton et al., 1985). The 2- and 8-azido nucleotides have also been used to map nucleotide binding domains of purified proteins (Khatoon et al., 1989; King et al., 1989; Dholakia et al., 1989) and may be used as antibody binding agents.

Several methods are known in the art for the attachment or conjugation of an antibody to its conjugate moiety. Some attachment methods involve the use of a metal chelate complex employing, for example, an organic chelating agent such a diethylenetriaminepentaacetic acid anhydride (DTPA); ethylenetriaminetetraacetic acid; N-chloro-p-toluenesulfonamide; and/or tetrachloro-3α-6α-diphenylglycouril-3 attached to the antibody (U.S. Pat. Nos. 4,472,509 and 4,938,948). Monoclonal antibodies may also be reacted with an enzyme in the presence of a coupling agent such as glutaraldehyde or periodate. Conjugates with fluorescein markers are prepared in the presence of these coupling agents or by reaction with an isothiocyanate. In U.S. Pat. No. 4,938,948, imaging of breast tumors is achieved using monoclonal antibodies and the detectable imaging moieties are bound to the antibody using linkers such as methyl-p-hydroxybenzimidate or N-succinimidyl-3-(4-hydroxyphenyl)propionate.

In other embodiments, derivatization of immunoglobulins by selectively introducing sulfhydryl groups in the Fc region of an immunoglobulin, using reaction conditions that do not alter the antibody combining site are contemplated. Antibody conjugates produced according to this methodology are disclosed to exhibit improved longevity, specificity and sensitivity (U.S. Pat. No. 5,196,066, incorporated herein by reference). Site-specific attachment of effector or reporter molecules, wherein the reporter or effector molecule is conjugated to a carbohydrate residue in the Fc region have also been disclosed in the literature (O'Shannessy et al., 1987). This approach has been reported to produce diagnostically and therapeutically promising antibodies which are currently in clinical evaluation.

### VI. IMMUNODETECTION METHODS

In still further embodiments, the present invention concerns immunodetection methods for binding, purifying, removing, quantifying and otherwise generally detecting influenza virus and its associated antigens. While such methods can be applied in a traditional sense, another use will be in quality control and monitoring of vaccine and other virus stocks, where antibodies according to the present invention can be used to assess the amount or integrity (i.e., long term stability) of H1 antigens in viruses. Alternatively, the methods may be used to screen various antibodies for appropriate/desired reactivity profiles.

Some immunodetection methods include enzyme linked immunosorbent assay (ELISA), radioimmunoassay (RIA), immunoradiometric assay, fluoroimmunoassay, chemiluminescent assay, bioluminescent assay, and Western blot to mention a few. In particular, a competitive assay for the detection and quantitation of influenza virus antibodies directed to specific parasite epitopes in samples also is provided. The steps of various useful immunodetection methods have been described in the scientific literature, such as, e.g., Doolittle and Ben-Zeev (1999), Gulbis and Galand (1993), De Jager et al. (1993), and Nakamura et al. (1987). In general, the immunobinding methods include obtaining a sample suspected of containing influenza virus, and contacting the sample with a first antibody in accordance with the present invention, as the case may be, under conditions effective to allow the formation of immunocomplexes.

These methods include methods for purifying influenza virus or related antigens from a sample. The antibody will preferably be linked to a solid support, such as in the form of a column matrix, and the sample suspected of containing the influenza virus or antigenic component will be applied to the immobilized antibody. The unwanted components will be washed from the column, leaving the influenza virus antigen immunocomplexed to the immobilized antibody, which is then collected by removing the organism or antigen from the column.

The immunobinding methods also include methods for detecting and quantifying the amount of influenza virus or related components in a sample and the detection and quantification of any immune complexes formed during the binding process. Here, one would obtain a sample suspected of containing influenza virus or its antigens, and contact the sample with an antibody that binds influenza virus or components thereof, followed by detecting and quantifying the amount of immune complexes formed under the specific conditions. In terms of antigen detection, the biological sample analyzed may be any sample that is suspected of containing influenza virus or influenza virus antigen, such as a tissue section or specimen, a homogenized tissue extract, a biological fluid, including blood and serum, or a secretion, such as feces or urine.

Contacting the chosen biological sample with the antibody under effective conditions and for a period of time sufficient to allow the formation of immune complexes (primary immune complexes) is generally a matter of simply adding the antibody composition to the sample and incubating the mixture for a period of time long enough for the antibodies to faun immune complexes with, i.e., to bind to influenza virus or antigens present. After this time, the sample-antibody composition, such as a tissue section, ELISA plate, dot blot or Western blot, will generally be washed to remove any non-specifically bound antibody species, allowing only those antibodies specifically bound within the primary immune complexes to be detected.

In general, the detection of immunocomplex formation is well known in the art and may be achieved through the application of numerous approaches. These methods are generally based upon the detection of a label or marker, such as any of those radioactive, fluorescent, biological and enzymatic tags. Patents concerning the use of such labels include U.S. Pat. Nos. 3,817,837, 3,850,752, 3,939,350, 3,996,345, 4,277,437, 4,275,149 and 4,366,241. Of course, one may find additional advantages through the use of a secondary binding ligand such as a second antibody and/or a biotin/avidin ligand binding arrangement, as is known in the art.

The antibody employed in the detection may itself be linked to a detectable label, wherein one would then simply detect this label, thereby allowing the amount of the primary immune complexes in the composition to be determined. Alternatively, the first antibody that becomes bound within the primary immune complexes may be detected by means of a second binding ligand that has binding affinity for the antibody. In these cases, the second binding ligand may be linked to a detectable label. The second binding ligand is itself often an antibody, which may thus be termed a “secondary” antibody. The primary immune complexes are contacted with the labeled, secondary binding ligand, or antibody, under effective conditions and for a period of time sufficient to allow the formation of secondary immune complexes. The secondary immune complexes are then generally washed to remove any non-specifically bound labeled secondary antibodies or ligands, and the remaining label in the secondary immune complexes is then detected.

Further methods include the detection of primary immune complexes by a two step approach. A second binding ligand, such as an antibody that has binding affinity for the antibody, is used to foam secondary immune complexes, as described above. After washing, the secondary immune complexes are contacted with a third binding ligand or antibody that has binding affinity for the second antibody, again under effective conditions and for a period of time sufficient to allow the formation of immune complexes (tertiary immune complexes). The third ligand or antibody is linked to a detectable label, allowing detection of the tertiary immune complexes thus formed. This system may provide for signal amplification if this is desired.

One method of immunodetection uses two different antibodies. A first biotinylated antibody is used to detect the target antigen, and a second antibody is then used to detect the biotin attached to the complexed biotin. In that method, the sample to be tested is first incubated in a solution containing the first step antibody. If the target antigen is present, some of the antibody binds to the antigen to form a biotinylated antibody/antigen complex. The antibody/antigen complex is then amplified by incubation in successive solutions of streptavidin (or avidin), biotinylated DNA, and/or complementary biotinylated DNA, with each step adding additional biotin sites to the antibody/antigen complex. The amplification steps are repeated until a suitable level of amplification is achieved, at which point the sample is incubated in a solution containing the second step antibody against biotin. This second step antibody is labeled, as for example with an enzyme that can be used to detect the presence of the antibody/antigen complex by histoenzymology using a chromogen substrate. With suitable amplification, a conjugate can be produced which is macroscopically visible.

Another known method of immunodetection takes advantage of the immuno-PCR (Polymerase Chain Reaction) methodology. The PCR method is similar to the Cantor method up to the incubation with biotinylated DNA, however, instead of using multiple rounds of streptavidin and biotinylated DNA incubation, the DNA/biotin/streptavidin/antibody complex is washed out with a low pH or high salt buffer that releases the antibody. The resulting wash solution is then used to carry out a PCR reaction with suitable primers with appropriate controls. At least in theory, the enormous amplification capability and specificity of PCR can be utilized to detect a single antigen molecule.

1. ELISAs

Immunoassays, in their most simple and direct sense, are binding assays. Certain preferred immunoassays are the various types of enzyme linked immunosorbent assays (ELISAs) and radioimmunoassays (RIA) known in the art. Immunohistochemical detection using tissue sections is also particularly useful. However, it will be readily appreciated that detection is not limited to such techniques, and western blotting, dot blotting, FACS analyses, and the like may also be used.

In one exemplary ELISA, the antibodies of the invention are immobilized onto a selected surface exhibiting protein affinity, such as a well in a polystyrene microtiter plate. Then, a test composition suspected of containing the influenza virus or influenza virus antigen is added to the wells. After binding and washing to remove non-specifically bound immune complexes, the bound antigen may be detected. Detection may be achieved by the addition of another anti-influenza virus antibody that is linked to a detectable label. This type of ELISA is a simple “sandwich ELISA.” Detection may also be achieved by the addition of a second anti-influenza virus antibody, followed by the addition of a third antibody that has binding affinity for the second antibody, with the third antibody being linked to a detectable label.

In another exemplary ELISA, the samples suspected of containing the influenza virus or influenza virus antigen are immobilized onto the well surface and then contacted with the anti-influenza virus antibodies of the invention. After binding and washing to remove non-specifically bound immune complexes, the bound anti-influenza virus antibodies are detected. Where the initial anti-influenza virus antibodies are linked to a detectable label, the immune complexes may be detected directly. Again, the immune complexes may be detected using a second antibody that has binding affinity for the first anti-influenza virus antibody, with the second antibody being linked to a detectable label.

Irrespective of the format employed, ELISAs have certain features in common, such as coating, incubating and binding, washing to remove non-specifically bound species, and detecting the bound immune complexes. These are described below.

In coating a plate with either antigen or antibody, one will generally incubate the wells of the plate with a solution of the antigen or antibody, either overnight or for a specified period of hours. The wells of the plate will then be washed to remove incompletely adsorbed material. Any remaining available surfaces of the wells are then “coated” with a nonspecific protein that is antigenically neutral with regard to the test antisera. These include bovine serum albumin (BSA), casein or solutions of milk powder. The coating allows for blocking of nonspecific adsorption sites on the immobilizing surface and thus reduces the background caused by nonspecific binding of antisera onto the surface.

In ELISAs, it is probably more customary to use a secondary or tertiary detection means rather than a direct procedure. Thus, after binding of a protein or antibody to the well, coating with a non-reactive material to reduce background, and washing to remove unbound material, the immobilizing surface is contacted with the biological sample to be tested under conditions effective to allow immune complex (antigen/antibody) formation. Detection of the immune complex then requires a labeled secondary binding ligand or antibody, and a secondary binding ligand or antibody in conjunction with a labeled tertiary antibody or a third binding ligand.

“Under conditions effective to allow immune complex (antigen/antibody) formation” means that the conditions preferably include diluting the antigens and/or antibodies with solutions such as BSA, bovine gamma globulin (BGG) or phosphate buffered saline (PBS)/Tween. These added agents also tend to assist in the reduction of nonspecific background.

The “suitable” conditions also mean that the incubation is at a temperature or for a period of time sufficient to allow effective binding. Incubation steps are typically from about 1 to 2 to 4 hours or so, at temperatures preferably on the order of 25° C. to 27° C., or may be overnight at about 4° C. or so.

Following all incubation steps in an ELISA, the contacted surface is washed so as to remove non-complexed material. A preferred washing procedure includes washing with a solution such as PBS/Tween, or borate buffer. Following the formation of specific immune complexes between the test sample and the originally bound material, and subsequent washing, the occurrence of even minute amounts of immune complexes may be determined.

To provide a detecting means, the second or third antibody will have an associated label to allow detection. Preferably, this will be an enzyme that will generate color development upon incubating with an appropriate chromogenic substrate. Thus, for example, one will desire to contact or incubate the first and second immune complex with a urease, glucose oxidase, alkaline phosphatase or hydrogen peroxidase-conjugated antibody for a period of time and under conditions that favor the development of further immune complex formation (e.g., incubation for 2 hours at room temperature in a PBS-containing solution such as PBS-Tween).

After incubation with the labeled antibody, and subsequent to washing to remove unbound material, the amount of label is quantified, e.g., by incubation with a chromogenic substrate such as urea, or bromocresol purple, or 2,2′-azino-di-(3-ethyl-benzthiazoline-6-sulfonic acid (ABTS), or H₂O₂, in the case of peroxidase as the enzyme label. Quantification is then achieved by measuring the degree of color generated, e.g., using a visible spectra spectrophotometer.

In another embodiment, the present invention contemplates the use of competitive formats. This is particularly useful in the detection of influenza virus antibodies in sample. In competition based assays, an unknown amount of analyte or antibody is determined by its ability to displace a known amount of labeled antibody or analyte. Thus, the quantifiable loss of a signal is an indication of the amount of unknown antibody or analyte in a sample.

Here, the inventors proposes the use of labeled influenza virus monoclonal antibodies to determine the amount of influenza virus antibodies in a sample. The basic format would include contacting a known amount of influenza virus monoclonal antibody (linked to a detectable label) with influenza virus antigen or particle. The influenza virus antigen or organism is preferably attached to a support. After binding of the labeled monoclonal antibody to the support, the sample is added and incubated under conditions permitting any unlabeled antibody in the sample to compete with, and hence displace, the labeled monoclonal antibody. By measuring either the lost label or the label remaining (and subtracting that from the original amount of bound label), one can determine how much non-labeled antibody is bound to the support, and thus how much antibody was present in the sample.

2. Western Blot

The Western blot (alternatively, protein immunoblot) is an analytical technique used to detect specific proteins in a given sample of tissue homogenate or extract. It uses gel electrophoresis to separate native or denatured proteins by the length of the polypeptide (denaturing conditions) or by the 3-D structure of the protein (native/non-denaturing conditions). The proteins are then transferred to a membrane (typically nitrocellulose or PVDF), where they are probed (detected) using antibodies specific to the target protein.

Samples may be taken from whole tissue or from cell culture. In most cases, solid tissues are first broken down mechanically using a blender (for larger sample volumes), using a homogenizer (smaller volumes), or by sonication. Cells may also be broken open by one of the above mechanical methods. However, it should be noted that bacteria, virus or environmental samples can be the source of protein and thus Western blotting is not restricted to cellular studies only. Assorted detergents, salts, and buffers may be employed to encourage lysis of cells and to solubilize proteins. Protease and phosphatase inhibitors are often added to prevent the digestion of the sample by its own enzymes. Tissue preparation is often done at cold temperatures to avoid protein denaturing.

The proteins of the sample are separated using gel electrophoresis. Separation of proteins may be by isoelectric point (pI), molecular weight, electric charge, or a combination of these factors. The nature of the separation depends on the treatment of the sample and the nature of the gel. This is a very useful way to determine a protein. It is also possible to use a two-dimensional (2-D) gel which spreads the proteins from a single sample out in two dimensions. Proteins are separated according to isoelectric point (pH at which they have neutral net charge) in the first dimension, and according to their molecular weight in the second dimension.

In order to make the proteins accessible to antibody detection, they are moved from within the gel onto a membrane made of nitrocellulose or polyvinylidene difluoride (PVDF). The membrane is placed on top of the gel, and a stack of filter papers placed on top of that. The entire stack is placed in a buffer solution which moves up the paper by capillary action, bringing the proteins with it. Another method for transferring the proteins is called electroblotting and uses an electric current to pull proteins from the gel into the PVDF or nitrocellulose membrane. The proteins move from within the gel onto the membrane while maintaining the organization they had within the gel. As a result of this blotting process, the proteins are exposed on a thin surface layer for detection (see below). Both varieties of membrane are chosen for their non-specific protein binding properties (i.e., binds all proteins equally well). Protein binding is based upon hydrophobic interactions, as well as charged interactions between the membrane and protein. Nitrocellulose membranes are cheaper than PVDF, but are far more fragile and do not stand up well to repeated probings. The uniformity and overall effectiveness of transfer of protein from the gel to the membrane can be checked by staining the membrane with Coomassie Brilliant Blue or Ponceau S dyes. Once transferred, proteins are detected using labeled primary antibodies, or unlabeled primary antibodies followed by indirect detection using labeled protein A or secondary labeled antibodies binding to the Fc region of the primary antibodies.

3. Immunohistochemistry

The antibodies of the present invention may also be used in conjunction with both fresh-frozen and/or formalin-fixed, paraffin-embedded tissue blocks prepared for study by immunohistochemistry (IHC). The method of preparing tissue blocks from these particulate specimens has been successfully used in previous IHC studies of various prognostic factors, and is well known to those of skill in the art (Brown et al., 1990; Abbondanzo et al., 1990; Allred et al., 1990).

Briefly, frozen-sections may be prepared by rehydrating 50 ng of frozen “pulverized” tissue at room temperature in phosphate buffered saline (PBS) in small plastic capsules; pelleting the particles by centrifugation; resuspending them in a viscous embedding medium (OCT); inverting the capsule and/or pelleting again by centrifugation; snap-freezing in −70° C. isopentane; cutting the plastic capsule and/or removing the frozen cylinder of tissue; securing the tissue cylinder on a cryostat microtome chuck; and/or cutting 25-50 serial sections from the capsule. Alternatively, whole frozen tissue samples may be used for serial section cuttings.

Permanent-sections may be prepared by a similar method involving rehydration of the 50 mg sample in a plastic microfuge tube; pelleting; resuspending in 10% formalin for 4 hours fixation; washing/pelleting; resuspending in warm 2.5% agar; pelleting; cooling in ice water to harden the agar; removing the tissue/agar block from the tube; infiltrating and/or embedding the block in paraffin; and/or cutting up to 50 serial permanent sections. Again, whole tissue samples may be substituted.

4. Immunodetection Kits

In still further embodiments, the present invention concerns immunodetection kits for use with the immunodetection methods described above. As the influenza virus antibodies are generally used to detect influenza virus or influenza virus antigens, the antibodies will be included in the kit. The immunodetection kits will thus comprise, in suitable container means, a first antibody that binds to influenza virus or influenza virus antigen, and optionally an immunodetection reagent.

In certain embodiments, the influenza virus antibody may be pre-bound to a solid support, such as a column matrix and/or well of a microtitre plate. The immunodetection reagents of the kit may take any one of a variety of forms, including those detectable labels that are associated with or linked to the given antibody. Detectable labels that are associated with or attached to a secondary binding ligand are also contemplated. Exemplary secondary ligands are those secondary antibodies that have binding affinity for the first antibody.

Further suitable immunodetection reagents for use in the present kits include the two-component reagent that comprises a secondary antibody that has binding affinity for the first antibody, along with a third antibody that has binding affinity for the second antibody, the third antibody being linked to a detectable label. As noted above, a number of exemplary labels are known in the art and all such labels may be employed in connection with the present invention.

The kits may further comprise a suitably aliquoted composition of the influenza virus or influenza virus antigens, whether labeled or unlabeled, as may be used to prepare a standard curve for a detection assay. The kits may contain antibody-label conjugates either in fully conjugated form, in the form of intermediates, or as separate moieties to be conjugated by the user of the kit. The components of the kits may be packaged either in aqueous media or in lyophilized form.

The container means of the kits will generally include at least one vial, test tube, flask, bottle, syringe or other container means, into which the antibody may be placed, or preferably, suitably aliquoted. The kits of the present invention will also typically include a means for containing the antibody, antigen, and any other reagent containers in close confinement for commercial sale. Such containers may include injection or blow-molded plastic containers into which the desired vials are retained.

### VII. EXAMPLES

The following examples are included to demonstrate preferred embodiments of the invention. It should be appreciated by those of skill in the art that the techniques disclosed in the examples which follow represent techniques discovered by the inventors to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

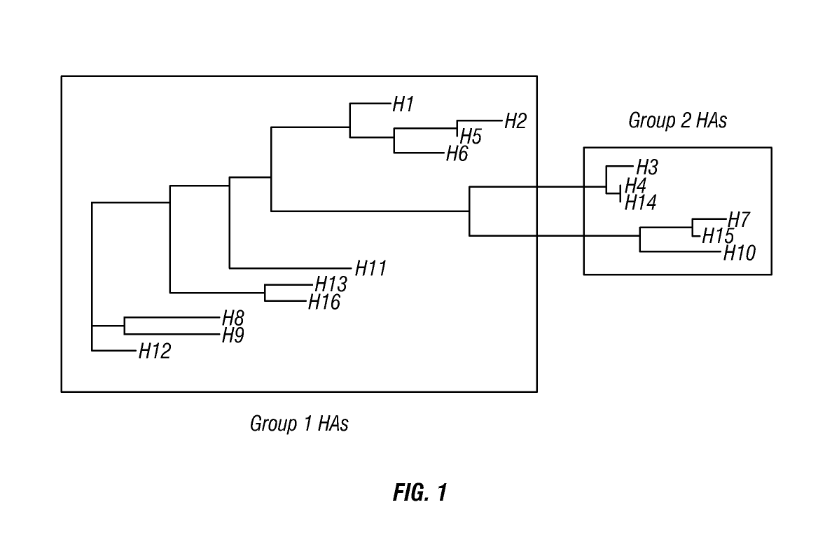

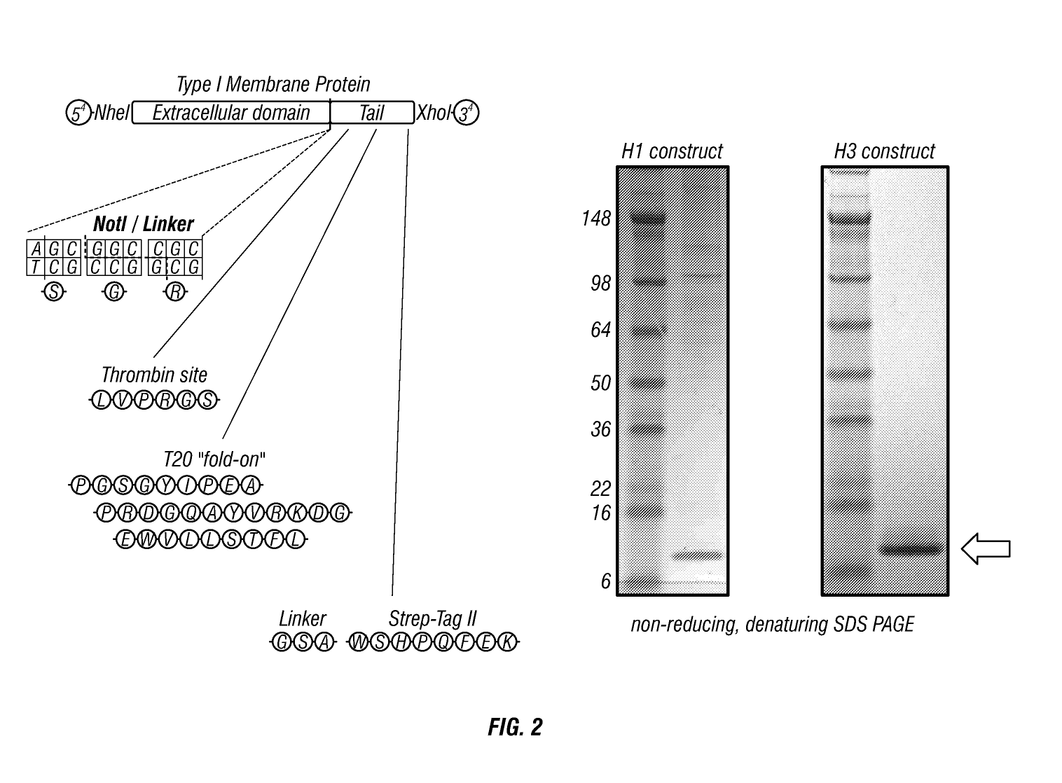

First-generation long alpha-helix (LAH) antigens. The 16 HA subtypes of influenza A fall into two phylogenetic groupings, Group 1 (H1, H2, H5, H6, H8, H9, H11, H12, H13, and H16) and Group 2 (H3, H4, H7, H10, H14, and H15). Within a group, there is a high sequence-identity within the influenza stalk region, suggesting that most stalk Abs broadly react within a phylogenetic group, but that reactivity across Group 1 and Group 2 HAs would be more difficult to achieve (FIG. 1). To screen for LAH Abs, the inventors synthesized two constructs based on the LAH of H1 HA, a representative of Group 1 HAs, and the LAH of H3 HA, a representative of Group 2 HAs. Those constructs were designated H1frThrST and H3frThrST with a shared architecture consisting of a modified mouse kappa leader sequence followed by the LAH domain (residues 76-130 of HA2), an SGR (serine, glycine, arginine) linker, a thrombin protease cleavage site, a T20 foldon trimerization domain, a GSA (glycine, serine, alanine) linker, and a StrepTag II site for purification. The constructs were cloned into pcDNA3.1 using the NheI and XhoI sites at the 5′ or 3′ ends, respectively (FIG. 2).

Second-generation LAH antigens. Using similar design principles, the inventors synthesized modified second-generation constructs with a GCN4 trimerization domain immediately following the LAH without any linkers or cleavage sites, but maintaining the periodicity of the heptad repeats. The trimerization domain is followed by a GSA (glycine, serine, alanine) linker and a One-STrEP-tag for purification followed by a GGGSGGGS (SEQ ID NO:109) linker and a C-terminal cysteine for coupling to adjuvants such as keyhole limpet hemocyanin (KLH) immunogen carrier protein.

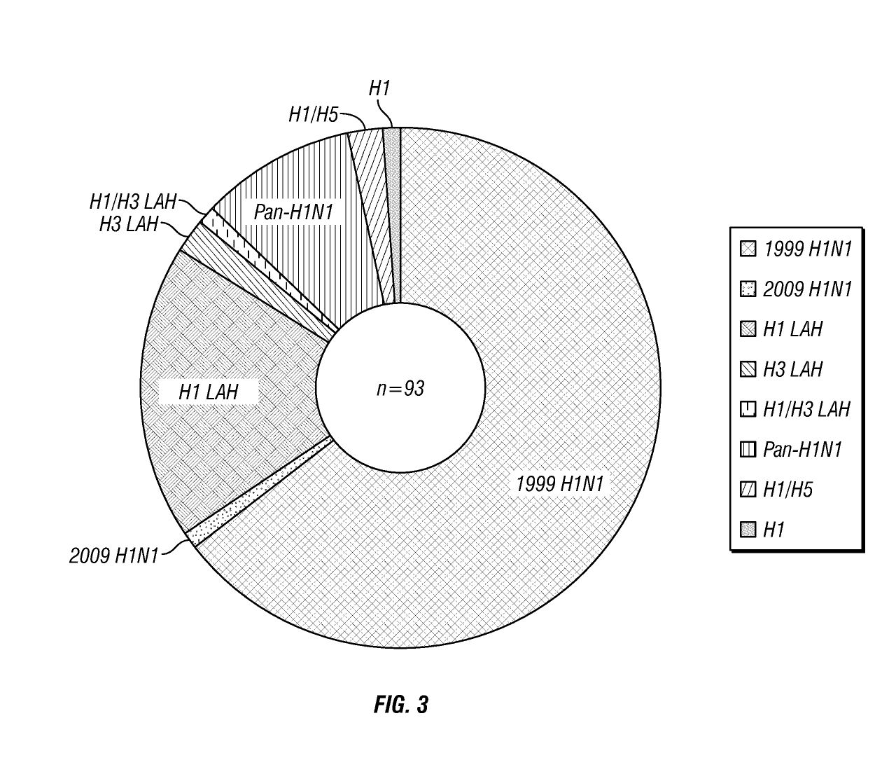

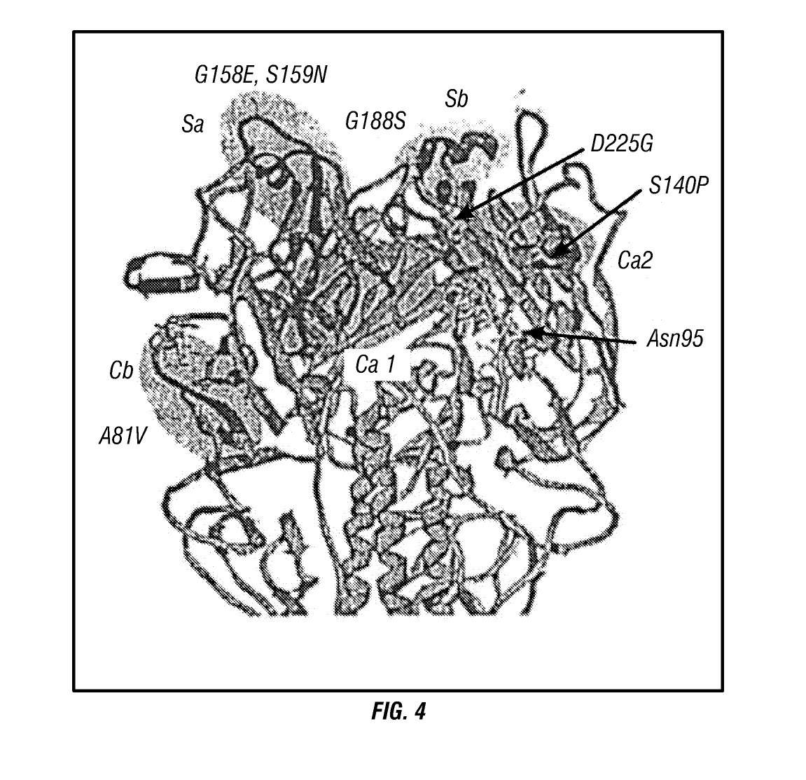

LAH-specific human Abs. The inventors used the first-generation LAH antigens to screen EBV-transformed B cells and hybridomas from influenza vaccinees and convalescents for reactivity. The patterns from screening donors were remarkable. One donor repertoire is shown in FIG. 3. They cloned LAH Abs from such donors. MAb 3N6, a human mAb against Group 1 HA LAH antigen, was the first such Ab to be molecularly cloned.

New LAH-specific Ab secreting hybridomas. Tables 1.1A-D are based on recombinant protein (A) or hybridoma supernatant (B-D) binding by ELISA to a panel of soluble HA proteins or first-generation LAH proteins. Recombinant mAb 3N6 binds Group 1 HAs and the LAH construct, as expected based on the corresponding hybridoma Ab (Table 1.1A). Table 1.1B lists new hybridomas that secrete Abs that, like mAb 3N6, recognize the Group 1 HAs and the corresponding H1 LAH construct. Table 1.1C lists the hybridomas 5J4 and 1C23 that recognize the H3 LAH and soluble pandemic 1968 HA, the representative of Group 2 influenza HAs here. The inventors have isolated hybridomas (Table 1.1D) that bind both Group 1 and Group 2 HAs and that may bind LAH of all 16 HAs; these are 6K14, 6J24, or 5K24. Table 1.2 shows additional LAH reactivity, including two additional antibodies and two antibodies from Table 1.1.

Non-canonical short alpha-helix (SAH) domain stalk Ab 10E15. The inventors identified hybridoma 10E15 as secreting an Ab that broadly reacted with Group 1 HAs, but not with the Group 1 LAH epitope (Table 2). This finding suggests a stalk-binding Ab that recognizes an epitope that is similar to that of the previously described V_{H}1-69 germline Abs, but sequence analysis of the heavy chain revealed a V_{H}3-30 germline instead. Likely this Ab encoded by an unusual, non-canonical Ab gene binds with differing structural features than the usual V_{H}1-69 mAbs previously described.

Canonical short alpha-helix domain stalk Abs using the V_{H}1-69 germline gene: mAbs 8D4 and 10A14. These mAbs were cloned from a 1918 influenza pandemic survivor or a 2009 H1N1 vaccinee, respectively. On cross-reactivity screening by ELISA, the Abs bound H1N1 HA and H5N1 HA, suggesting a Group 1 stalk-binding pattern. DNA sequencing revealed a V_{H}1-69 germline gene background of those Abs similar to the previously described Abs CR6261 and F10. Sequence alignment of those Abs, however, suggests differences in the new Abs as compared to CR6261 and F10, such as the 3 commonly mutated amino acid residues within the framework three region, as well as similarities such as a tyrosine in a similar position of CDR-H3. Recombinant Ab based on the cloned sequences confirmed the pattern of reactivity seen with the hybridoma supernatants. In recent work, the inventors isolated about a dozen more of the V_{H}1-69 Abs (not shown).

TABLE 1.1
Long alpha-helix (LAH) specific human Abs and their reactivity patterns

| | | | | | | | | | |
|---|---|---|---|---|---|---|---|---|---|
| | | 1918 | 1957 | 1968 | 1999 | 2004 | 2009 | H1 | |
| Hybridoma | Donor | H1 | H2 | H3 | H1 | H5 | H1 | LAH | H3 LAH |

A) Group 1 specific recombinant mAb 3N6

| | | | | | | | | | |
|---|---|---|---|---|---|---|---|---|---|
| 3N6 recomb | S11 SFD | 0.126 | 0.570 | 0.069 | 0.909 | 2.029 | 1.033 | 0.910 | 0.072 |

B) Group 1 specific mAbs

| | | | | | | | | | |
|---|---|---|---|---|---|---|---|---|---|
| 5F18 | | 0.224 | 0.691 | 0.067 | 0.896 | 1.096 | 1.778 | 1.188 | 0.062 |
| 13H19 | S18 H5 | 0.079 | 0.407 | 0.073 | 0.119 | 1.093 | 0.676 | 0.607 | 0.069 |
| 1O19 | S29 H5 | 0.128 | 0.533 | 0.069 | 0.554 | 1.483 | 1.322 | 1.878 | 0.070 |
| 5O20 | | 0.170 | 0.618 | 0.067 | 0 519 | 1.200 | 1.560 | 2.083 | 0.063 |
| 1B14 | S36 H5 | 0.066 | 1.261 | 0.069 | 0.710 | 1.791 | 2.249 | 2.251 | 0.082 |
| 3D19 | S42 H5 | 0.174 | 0.749 | 0.071 | 0.678 | 1.582 | 1.534 | 1.683 | 0.071 |
| 3B3 | | 0.090 | 1.579 | 0.071 | 1.545 | 2.059 | 2.376 | 2.200 | 0.068 |
| 6I19 | | 0.068 | 0.099 | 0.072 | 0.105 | 0.234 | 0.207 | 0.175 | 0.067 |
| 1K12 | | 0.089 | 0.537 | 0.073 | 0.959 | 2.087 | 1.397 | 2.000 | 0.071 |
| 2F18 | S54 H5 | 0.511 | 1.247 | 0.066 | 1.328 | 1.843 | 1.747 | 1.740 | 0.068 |
| 17F16 | S7 Cro53 | 0.173 | 0.151 | 0.071 | 0.553 | 0.983 | 0.812 | 1.058 | 0.068 |

C) Group 2 specific mAbs

| | | | | | | | | | |
|---|---|---|---|---|---|---|---|---|---|
| 5J4 | S11 SFD | 0.072 | 0.074 | 0.748 | 0.077 | 0.073 | 0.081 | 0.075 | 2.311 |
| 1C23 | S13 Cro53 | 0.073 | 0.075 | 0.507 | 0.082 | 0.076 | 0.084 | 0.071 | 1.131 |

D) Group 1 & 2 reactive mAbs

| | | | | | | | | | |
|---|---|---|---|---|---|---|---|---|---|
| 6K14 | S29 H5 | 0.155 | 1.933 | 0.135 | 0.203 | 1.123 | 2.119 | 2.346 | 2.447 |
| 6J24 | | 0.072 | 0.219 | 0.073 | 0.183 | 0.379 | 0.981 | 0.418 | 0.940 |
| 5K24 | S42 H5 | 0.082 | 0.126 | 0.126 | 0.129 | 0.154 | 0.302 | 0.237 | 0.217 |
| 5F20 | S11 SFD | 0.170 | 0.075 | 0.065 | 0.192 | 0.330 | 0.315 | 0.514 | 0.203 |

Cell supernates were tested by ELISA. Values shown are OD. Yellow indicates positive signal for binding.

TABLE 1.2
Cross-reactive long alpha-helix (LAH) specific human Abs and their reactivity patterns

| | 1918 | 1957 | 1968 | 1999 | 2004 | 2009 | H1 | | |
|---|---|---|---|---|---|---|---|---|---|
| Hybridoma | H1 | H2 | H3 | H1 | H5 | H1 | LAH | 1997 H6N1 | 1997 H9N2 |
| 5117 (K23/F21) | 0.522 | 1.379 | 0.156 | 1.192 | 1.169 | 0.566 | 0.078 | 0.110 | 1.982 |
| 6J24 (M19) | 0.083 | 0.097 | 0.078 | 0.083 | 0.182 | 0.102 | 0.189 | 0.163 | 0.075 |
| 3N6 recomb | 0.107 | 0.801 | 0.083 | 1.488 | 3.069 | 2.693 | 3.263 | 0.230 | 0.084 |
| 5G11 (C5/M7) | 0.128 | 0.076 | 0.142 | 0.649 | 1.874 | 0.374 | 0.079 | 0.358 | 1.063 |

Cell supernates were tested by ELISA. Values shown are OD. Yellow indicates positive signal for binding.

TABLE 2
The non-V_{H}1-69 short alpha-helix specific human Ab 10E15 and its reactivity patterns

| | | 1918 | 1957 | 1968 | 1999 | 2004 | 2009 | H1 | |
|---|---|---|---|---|---|---|---|---|---|
| Hybridoma | Donor | H1 | H2 | H3 | H1 | H5 | H1 | LAH | H3 LAH |
| 10E15 | S9 Cro53 | 0.106 | 0.485 | 0.074 | 0.514 | 1.693 | 0.996 | 0.068 | 0.070 |

Cell supernates were tested by ELISA. Values shown are OD. Yellow indicates positive signal for binding.

### Example 2

Pan-HA mAbs that bind both Group 1 and Group 2 HAs: 3E22 and 5I17. Screening hybridomas from an H5 vaccinee with soluble H5 HA and subsequently on a cross-reactivity ELISA (Table 3), the inventors isolated an unprecedented binding pattern for two hybridomas, 3E22 and 5I17: They bound all soluble HAs on the panel, all Group 1 HAs (H1, H2, H5), and also the Group 2 HA (H3). The lack of detectable binding to the LAH constructs suggested they were binding to the head domain. Consistent with this, the mAb 3E22 exhibited an HAI activity of 488 ng/mL, further suggesting that this class of Abs binds a conserved domain on the globular head that has not been previously recognized. These are the most broadly reactive flu mAbs ever isolated.

Pan-H1N1 Abs. The inventors identified a mAb, 8F24, generated from B-cells of a human survivor of the 1918 influenza pandemic. 8F24 recognized all H1N1 strains that the inventors tested with and showed reactivity against H3 HA at high concentrations, but did otherwise not have cross-heterosubtypic activity on ELISA testing (Table 4). This finding suggests that this Ab binds a conserved epitope on the globular head of HA that potentially includes part of the receptor-binding pocket.

Abs with a pan-H1 ELISA binding pattern similar to 8F24. The inventors isolated 7 additional mAbs from 3 different donors (Table 5); these novel Abs showed a similar pattern of binding as mAb 8F24, specifically binding all H1N1 HAs, but neither of these Abs demonstrated binding to soluble 1968 H3 HA as unconcentrated supernates. Nevertheless, it is highly likely that these Abs all recognize the same epitope.

Pandemic (1918 H1N1, 1957 H2N2, 1968 H3N2) or seasonal (1999 H1N1) virus specific mAbs. In the course of screening influenza immune individuals, the inventors have identified mAbs that exhibit specificity for particular pandemic viruses, or the 1999 H1 seasonal virus HA (a component of vaccine for about 10 years). They are almost certainly specific for antigenic loops on the head domain. Of course, these mAbs are in some ways less exciting than many described above because of their lack of heterosubtypic cross-reactivity. Nevertheless, these represent the first human Abs directed to the viruses, and thus the inventors believe they are of some interest. In particular, defining elements of the HA head domain that are not conserved across different HA subtypes will help us to more precisely define the borders of the cross-reactive epitopes recognized by the heterosubtypic mAbs. The inventors have previously described a panel of 1918 virus specific mAbs (Yu et al., 2008). These Abs do not recognize H1 HAs after the 1930s. The inventors have recently isolated mAbs recognizing only the 1957 H2N2 pandemic flu HA (Table 6), only the 1968 H3N2 pandemic flu HA (Table 7), or only the seasonal 1999 H1N1 (Table 8), from subjects born between 1957-68.

TABLE 3
Head domain specific human mAb that bind all HAs tested, from both Group 1 & 2

| | | 1918 | 1957 | 1968 | 1999 | 2004 | 2009 | H1 | |
|---|---|---|---|---|---|---|---|---|---|
| Hybridoma | Donor | H1 | H2 | H3 | H1 | H5 | H1 | LAH | H3 LAH |
| 3E22 | | 0.741 | 1.178 | 0.379 | 2.063 | 1.571 | 2.379 | 0.087 | 0.086 |
| 5I17 | S42 H5 | 0.495 | 0.850 | 0.267 | 1.931 | 1.548 | 0.668 | 0.070 | 0.070 |
| 5G11 (O22) | | 0.274 | 0.067 | 0.363 | 0.464 | 0.618 | 0.299 | 0.066 | 0.083 |

Cell supernate was tested by ELISA. Values shown are OD. Yellow indicates positive signal for binding.

TABLE 4
Head domain specific pan-H1 human mAb 8F24 and
its reactivity patterns

| | 1918 | 1957 | 1968 | 1999 | 2004 | 2009 | H1 | |
|---|---|---|---|---|---|---|---|---|
| Donor | H1 | H2 | H3 | H1 | H5 | H1 | LAH | H3 LAH |
| S6 Cro18 | 0.209 | 0.073 | 0.669 | 1.010 | 0.074 | 0.714 | 0.073 | 0.075 |

Cell supernate was tested by ELISA. Values shown are OD. Yellow indicates positive signal for binding.

TABLE 5
Head domain specific human mAb similar to mAb 8F24 and their reactivity patterns

| | | 1918 | 1957 | 1968 | 1999 | 2004 | 2009 | H1 | |
|---|---|---|---|---|---|---|---|---|---|
| Hybridoma | Donor | H1 | H2 | H3 | H1 | H5 | H1 | LAH | H3 LAH |
| 12F14D7 | S9 Cro53 | 0.245 | 0.080 | 0.072 | 0.498 | 0.074 | 1.598 | 0.098 | 0.070 |
| 13H6G16 | S13 Cro53 | 0.408 | 0.070 | 0.070 | 1.292 | 0.079 | 0.124 | 0.074 | 0.071 |
| 5J8P13 | S20 Cro53 | 0.262 | 0.077 | 0.078 | 1.458 | 0.077 | 1.928 | 0.076 | 0.083 |
| 1A17C19 | | 0.383 | 0.073 | 0.070 | 0.689 | 0.070 | 1.014 | 0.076 | 0.079 |
| 2K11E10 | | 0.311 | 0.080 | 0.080 | 0.518 | 0.075 | 1.285 | 0.079 | 0.072 |
| 2O10E6 | | 0.308 | 0.073 | 0.077 | 0.872 | 0.072 | 1.617 | 0.072 | 0.074 |
| 4A10D9 | | 0.725 | 0.072 | 0.072 | 1.377 | 0.075 | 2.261 | 0.069 | 0.073 |

Cell supernates were tested by ELISA. Values shown are OD. Yellow indicates positive signal for binding.

TABLE 6
1957 H2N2 pandemic flu HA mAbs

| Subject | mAb |
|---|---|
| 7 | 17L8 |
| | 18E6 |
| | 8G6 |
| 9 | 25F7 |
| | 8F8 |
| 13 | 2H22 |
| | 8K20 |
| 14 | 4I4 |
| 18 | 4E2 |

TABLE 7
1968 H3N2 pandemic flu HA mAbs

| Subject | mAb |
|---|---|
| 7 | 7A13 |
| 12 | 2L15 |
| 13 | 11J19 |
| | 15C13 |

TABLE 8
1999 H1N1 seasonal flu HA mAbs

| Subject | mAb |
|---|---|
| 7 | 3J10 |
| 11 | 11I12 |
| | 12D7 |
| | 5I5 |
| 20 | 12I1 |
| | 2C7 |
| | 2L7 |

TABLE 9
Strain-Specific mAbs

| | | 1918 | 1957 | 1968 | 1999 | 2004 | 2009 | H1 | |
|---|---|---|---|---|---|---|---|---|---|
| Hybridoma | Donor | H1 | H2 | H3 | H1 | H5 | H1 | LAH | H3 LAH |
| 17L8 | S7 Cro53 | 0.069 | 0.838 | 0.069 | 0.069 | 0.072 | 0.076 | 0.066 | 0.073 |
| 18E6 | S7 Cro53 | 0.063 | 0.305 | 0.068 | 0.067 | 0.072 | 0.068 | 0.068 | 0.066 |
| 18G6 | S7 Cro53 | 0.075 | 0.757 | 0.073 | 0.071 | 0.071 | 0.071 | 0.071 | 0.071 |
| 25F7 | S9 Cro53 | 0.075 | 0.871 | 0.085 | 0.077 | 0.076 | 0.078 | 0.072 | 0.073 |
| 8F8 | S9 Cro53 | 0.070 | 1.025 | 0.079 | 0.081 | 0.074 | 0.081 | 0.071 | 0.071 |
| 8K20 | S13 Cro53 | 0.125 | 1.276 | 0.070 | 0.071 | 0.075 | 0.071 | 0.068 | 0.068 |
| 2H22 | S13 Cro53 | 0.069 | 1.500 | 0.072 | 0.072 | 0.087 | 0.081 | 0.071 | 0.069 |
| 4E2 | S18 Cro53 | 0.072 | 0.656 | 0.077 | 0.070 | 0.075 | 0.081 | 0.073 | 0.069 |
| 7A13 | S7 Cro53 | 0.076 | 0.070 | 0.265 | 0.069 | 0.076 | 0.069 | 0.068 | 0.068 |
| 25J3 | S9 Cro53 | 0.070 | 0.070 | 0.623 | 0.071 | 0.070 | 0.071 | 0.068 | 0.067 |
| 2L15 | S12 Cro53 | 0.069 | 0.075 | 0.183 | 0.072 | 0.074 | 0.076 | 0.069 | 0.071 |
| 15C13 | S13 Cro53 | 0.072 | 0.084 | 1.003 | 0.072 | 0.075 | 0.076 | 0.068 | 0.069 |
| 11J19 | S13 Cro53 | 0.079 | 0.079 | 0.895 | 0.071 | 0.067 | 0.081 | 0.085 | 0.075 |
| 3J10 | S7 Cro53 | 0.069 | 0.074 | 0.076 | 0.711 | 0.075 | 0.073 | 0.070 | 0.069 |
| 5I5 | S11 SFD | 0.071 | 0.073 | 0.072 | 1.770 | 0.070 | 0.071 | 0.071 | 0.073 |
| 12D7 | S11 SFD | 0.072 | 0.089 | 0.083 | 1.342 | 0.069 | 0.074 | 0.068 | 0.069 |
| 11I12 | S11 SFD | 0.075 | 0.073 | 0.069 | 1.287 | 0.068 | 0.079 | 0.070 | 0.069 |
| 12I1 | S20 Cro53 | 0.065 | 0.075 | 0.071 | 0.758 | 0.068 | 0.084 | 0.072 | 0.068 |
| 2L7 | S20 Cro53 | 0.070 | 0.072 | 0.071 | 1.492 | 0.071 | 0.072 | 0.069 | 0.071 |
| 2C7 | S20 Cro53 | 0.067 | 0.068 | 0.070 | 1.228 | 0.138 | 0.073 | 0.070 | 0.071 |

### Example 3

### A. Methods

Hybridoma generation and recombinant antibody expression. Peripheral blood mononuclear cells (PBMCs) were isolated, Epstein Barr virus-transformed in 384 well plates (Nunc) in the presence of 2.5 μg/mL CpG ODN 2006 (Invivogen), 10 μM Chk2 inhibitor II (Sigma C3742), and 1 μg/mL cyclosporine A (Sigma), essentially as previously described Yu et al., 2008b; Yu et al., 2008a. Supernatants from wells containing EBV-transformed lymphoblastoid cell lines were screened for binding activity by ELISA against a panel of recombinant soluble HA proteins. Positive wells were fused with HMMA2.5 myeloma cells to generate hybridoma cell lines that were cloned biologically by limiting dilution, and then the antibody genes were cloned molecularly by RT-PCR using previously described primer sets (Smith et al., 2009; Sblattero and Bradbury, 1998; Lagerkvist et al., 1995) into pGEM-T Easy vector (Promega) and eventually into pEE12.4/pEE6.4 mammalian expression vectors (Lonza). The immunoglobulin encoding plasmids were transfected into 293F cells and expressed (Xu et al., 2010b). Then, the antibody proteins were FPLC-purified using an ÅKTA chromatography instrument and MabSelect Sure affinity columns (GE).

Purification of antibodies from hybridoma cell line supernatants. The hybridoma cell line was thawed rapidly (for S139/1) and grown in medium E (STEMCELL Technologies) until resuspension of the cells in Hybridoma-SFM medium (Invitrogen). Supernatant was harvested after one week, FPLC-purified with protein G (for S139/1 or hybridoma-derived 2G1) or MabSelect SuRe affinity columns (for all other antibodies, both GE), and concentrated with Amicon Ultra centrifugal filters with a 30 kD molecular weight cut-off (Millipore).

Virus-like particle (VLP) expression and hemagglutination inhibition (HAI) assays. Expression plasmids encoding HA or neuraminidase proteins were co-expressed in 293T cells to produce VLPs (Yu et al., 2008a; Chen et al., 2007). Two days post-transfection, supernatants were collected. HAI assays were performed as described (Centers For Disease Control and Prevention) using VLPs or live virus, as indicated in the tables. The inventors used the numbering scheme from Xu et al. for H2 HA (PDB: 3KU3/3KU5/3KU6) (Xu et al., 2010a).

Generation and purification of recombinant soluble HA molecules. A cDNA encoding the full-length A/Japan/305+/1957 virus HA protein (GenBank accession number AAA64362) was sequence-optimized for expression in human cells and synthesized (GenScript). The extracellular domain was amplified using PCR and cloned into a vector containing a thrombin site, a fibritin trimerization domain, and a 6× histidine tag (Stevens et al., 2004). A 1968 H3 HA construct was sequence-optimized for expression in human cells and synthesized (GeneArt), based on the extracellular domain of the HA gene from A/Aichi/2/1968, a GCN4 trimerization domain, a TEV protease recognition site, and a 6× histidine tag. Both constructs were expressed in a pcDNA3.1(+) vector (Invitrogen) in 293F cells (Invitrogen), purified over nickel columns using an ÅKTA chromatography instrument (GE), and concentrated with Amicon filters as described above.

Enzyme linked immunosorbent assay (ELISA). 384-well clear plates (Nunc 242757) were coated with HA at 1 μg/mL in D-PBS overnight, blocked with 0.5% cows milk, 0.2% goat serum, and 0.05% TWEEN 20 (Sigma P7949) in D-PBS. Five μL of hybridoma supernatant per well were transferred to 25 μL of blocking solution with a multi-channel pipettor. Secondary AP-conjugated goat anti-human IgG antibodies (Meridian Life Science W99008A) were diluted 1:8,000 in blocking solution and added after four automated washing steps. After another wash, phosphatase substrate (Sigma S0942) was dissolved in substrate buffer per the instructions of the manufacturer and dispensed onto the plates. The optical density of solution in plates was read at 405 nm on a PowerWave HT (BioTek).

Isolation and characterization of antibody escape mutant viruses. The inventors selected new antibody escape mutant viruses by incubating virus with neutralizing antibodies followed by inoculation of the mixture in 10-day-old embryonated chicken eggs, essentially as described (Caton et al., 1982; Yewdell et al., 1979). RNA was extracted from virus-infected allantoic fluid, then cDNA was generated by RT-PCR, cloned molecularly, and sequenced.

In vivo antiviral effect of H2N2-specific mAbs. Female 8-week-old BALB/c mice were inoculated intranasally with 5×LD₅₀ in a 50 μL volume of the virulent A/Albany/6/1958 H2N2 influenza virus (Pappas et al., 2010; Viswanathan et al., 2010). At 24 h after inoculation, mice were each administered 200, 20, or 2 μg (approximately 10, 1, or 0.1 mg/kg) of Ab 8F8, 8M2, or 2G1 or an equal volume of 10 mg/kg of polyclonal human IgG (Sigma) by the i.p. route in groups of 10 mice. Mice were observed for weight loss for 14 days. Subsets of four animals treated with Abs were euthanized on day 4 after inoculation, and whole lungs were homogenized in 1 mL of sterile PBS. Virus titers in lung tissue homogenates were determined by plaque titration in Madin-Darby Canine Kidney cell monolayer cultures and expressed as log₁₀ PFU/mL. Statistics were performed with GNU R 2.13.1 (The R Foundation for Statistical Computing). The log-rank test was used to compare the survival distributions. The Wilcoxon rank sum test was used to compare the lung virus titers.

Review of H2N2 sequences. The inventors queried the Influenza Research Database (IRD; world-wide-web at fludb.org) on Apr. 29, 2011 for all naturally-occurring, non-redundant human H2N2 HA sequences between 1957 and 1968 to identify the variability of key residues. After an alignment with ClustalW (Larkin et al., 2007), complete sequences were pruned to residues 59-252 (encoding the “globular head”) of the HA1 subunit using MacVector 12 software. Redundant sequences were eliminated with a redundancy threshold of 100 in Jalview 2.6.1. A phylogram was generated with MacVector 12 using neighbor joining, best tree, symmetric tie breaking, uncorrected (“p”) distance settings, and rooted to A/Japan/305/1957 (CY014976).

Human and animal studies. All clinical investigation was conducted according to Declaration of Helsinki principles. Acquisition of human blood samples was approved by the Vanderbilt University Institutional Review Board, and informed consent was received from participants prior to inclusion in the study. The animal studies were approved by the Institutional Review Boards of the Centers for Disease Control.

### B. Results

Hybridoma generation and molecular cloning. The inventors screened peripheral blood cells from a total of 26 healthy donors born between 1957 and 1968 and from three donors who participated in an NIH-sponsored clinical trial of an experimental monovalent subvirion H5N1 influenza vaccine (Bernstein et al., 2008) by testing the supernatants of EBV-transformed B cells for antibodies binding to recombinant A/Japan/305+/1957 H2N2 HA or A/Aichi/2/1968 H3 HA by ELISA. Lymphoblastoid cell lines from wells with supernatants containing HA-reactive antibodies were fused with HMMA2.5 myeloma cells to generate hybridomas. Five antibodies were cloned from different donors. MAbs 8F8 and 8M2 reacted with the pandemic H2N2 HA, mAbs 7A13 and 11J19 recognized the pandemic H3N2 HA and, remarkably, mAb 2G1 reacted with both the pandemic H2N2 and H3N2 HA (Table 10). Molecular cloning of the antibodies and recombinant expression confirmed that the antibody gene sequences coded for HA-reactive antibody proteins. Nucleotide sequence analysis of variable gene sequences (Table 10) using the international ImMunoGeneTics (IMGT) information system (Brochet et al., 2008) revealed that the H2 mAbs 8M2 and 2G1 shared the V_{H}1-69 germline gene.

TABLE 10
Genetic features of H2/N3 influenza-specific human mAbs. 8F8, 8M2,
and 2G1 reacted with H2 HA; 2G1, 7A13, and 11J19 reacted with
H3 HA. Donor age is stated for the time of the blood draw (2006 for
subject 92, 2010 for the other subjects)

| | | Age | Heavy chain | | | Light chain | | |
|---|---|---|---|---|---|---|---|---|
| mAb | Subject | [yrs] | V_{H} | D | J_{H} | V_{L}/V_{K} | J_{L}/J_{K} | λ/κ |
| 8F8 | 9 | 51 | 3- | 5- | 6*02 | 1-44*01 | 2*01 or | λ |
| | | | 33*01 | 12*01 | | | 3*01 | |
| 8M2 | 16 | 54 | 1- | 3- | 4*02 | 3-15*01 | 1*01 | κ |
| | | | 69*01 | 16*02 | | | | |
| 2G1 | 92 | 56 | 1- | 1- | 5*02 | 1-33*01 | 5*01 | κ |
| | | | 69*01 | 26*01 | | or 1D- | | |
| | | | | | | 33*01 | | |
| 7A13 | 7 | 50 | 4-4*02 | 2- | 1*01 | 4-69*01 | 3*02 | λ |
| | | | | 21*02 | | | | |
| 11J19 | 13 | 45 | 4- | 3- | 4*02 | 1-9*01 | 4*01 | κ |
| | | | 39*01 | 10*01 | | | | |

Reactivity of H2 antibodies. The inventors tested the three H2 HA-reactive antibodies in hemagglutination inhibition (HAI) assays against a panel of representative influenza strains. MAbs 8F8, 8M2, and 2G1 each inhibited several H2 strains suggesting that they targeted the HA globular head. This specificity of 8M2 and 2G1 was surprising given the previously reported strong association of HA stem specificity with the use of the V_{H}1-69 germline gene segment in antibodies to influenza (Ekiert et al., 2009; Corti et al., 2010; Wrammert et al., 2011; Sui et al., 2009). MAb 8F8 or mAb 2G1 both potently inhibited all H2 strains tested except for a virus circulating in 1967 (Table 11). MAb 8M2 inhibited all strains tested including the virus from 1967, but did not react with the Japan/305/57 strain. Interestingly, mAbs 8F8, 8M2, and 2G1 each inhibited a swine H2N3 influenza strain from 2006 (Table 11).

TABLE 11
Specific HAI activity of antibodies against influenza viruses or VLPs (where indicated)

| | HAI activity against mAb [μg/mL] | | | | | | | | | | | |
|---|---|---|---|---|---|---|---|---|---|---|---|---|
| | H2N2 | | | | | | | H2N3 | | | | |
| | A/ | A/ | | A/ | | | | A/swine/ | H3N2 | | H1N1 | H5N1 |
| | Singapore/ | Japan/ | A/Ann | Rockville | A/ | A/ | A/Ann | Missouri/ | A/ | A/ | A/ | A/ |
| | 1/1957 | 305/305/ | Arbor/ | Illinois/ | Albany/ | Netherlands/ | Arbor/ | 2124514/ | Hong Kong/ | Alabama/ | California/ | Vietnam/ |
| mAb | (VLP) | 1957 | 23/1957 | 5/1957 | 6/1958 | 65/1963 | 7/1967 | 2006 | 1/1968 | 1/1981 | 04/2009 | 1203/2004 |
| 8F8 | 0.05 | 0.4 | 0.1 | 0.4 | 0.32 | 0.1 | > | 0.63 | > | NT | > | > |
| 8M2 | 0.2 | > | 0.7 | 0.2 | 1.25 | 0.7 | 10 | 1.25 | > | NT | > | > |
| 2G1 | 0.1 | 0.7 | 0.025 | 0.2 | 0.32 | 0.05 | > | 0.63 | 5 | >> | >> | >> |
| 7A13 | > | NT | NT | NT | NT | NT | NT | NT | 0.7 | > | > | > |
| 11J19 | > | NT | NT | NT | NT | NT | NT | NT | 2.5 | > | > | > |
| murine | >> | NT | NT | NT | NT | NT | NT | NT | <0.2 | >> | >> | >> |
| S139/1^{†} | | | | | | | | | | | | |

^{†}S139/1 is a previously published murine hybridoma mAb (Yoshida et al., 2009).
The highest titer that still showed HAI activity is stated.
NT = not tested.
The > symbol indicates that activity was not detected at the highest concentration tested, 20 μg/mL. We tested select antibodies at up to 50 μg/mL;
the >> symbol denotes no activity detected at this higher concentration.

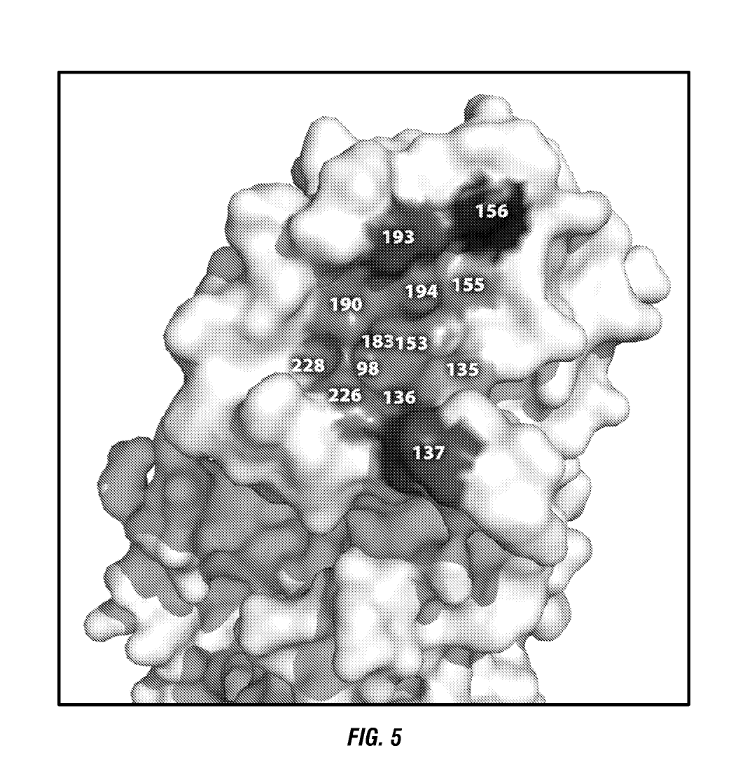

Epitope mapping of H2N2 antibodies. Next, the inventors sought to understand why mAb 8M2 did not neutralize the Japan/305/57 strain. Japan/305/57 has an avian receptor-specificity. To test whether a residue that mediated this receptor-specificity also mediated escape from 8M2 inhibition, the inventors made H2N2 virus-like particles (VLPs) using the cDNA from the HA of Japan/305+/57 virus, which exhibits human receptor-specificity. As expected, mAb 8M2 inhibited the HA activity of these VLPs (Table 12). The sequence of the Japan/305+/57 virus differs from that of Japan/305/57 mainly in a serine residue at position 228 instead of a glycine (FIG. 5). When the inventors reverted this serine back to a glycine in the Japan/305+/57 HA and used the cDNA to make VLPs, inhibition of those VLPs by mAb 8M2 was reduced markedly (Table 12).

TABLE 12
Specific HAI activity of human H2N2 antibodies against wild-type or
mutated VLPs

| | HAI activity against mAb [μg/ml] | | | | | | | | | |
|---|---|---|---|---|---|---|---|---|---|---|
| | H2N2 VLPs | | | | | | | | | |
| mAb | wt | G135D | G135S | R137Q | R137M | R137K | K156E | T193K | T193A | S228G |
| 8F8 | <0.2 | 0.7 | <0.2 | 10 | > | 10 | 0.1 | > | <0.2 | 0.7 |
| 8M2 | 0.7 | > | 1.3 | 0.7 | 1.3 | 0.7 | 0.4 | 10 | 0.7 | 2.5 |
| 2G1 | <0.2 | 0.7 | <0.2 | <0.2 | <0.2 | <0.2 | > | 1.3 | <0.2 | 0.4 |

The highest titer that still showed HAI activity is stated.
The > symbol indicates that activity was not detected at the highest concentration tested, 20 μg/mL.
VLPs are based on the HA of A/Japan/305+/1957 except K156 which is based on A/Singapore/1/1957.

To more fully define the antibody epitopes, the inventors generated escape mutant viruses, using the rationale that sequence polymorphisms in escape mutants will reflect the epitopes recognized by the mAb. MAb 8M2 selected for a G135D mutation in the Singapore/57 virus background, a residue located on the edge of the RBD opposite of residue 228 implicated above (FIG. 5). MAb 8F8 selected for a T193K mutation in the Singapore/57 background and an R137Q mutation in the Japan/305/57 background. Finally, mAb 2G1 elicited a K156E mutation in the Singapore/57 background. These residues at positions 137 and 156/193 also are located at the edge of the RBD on opposite sides of the structure, but at an approximate 90° rotation compared to the axis of the 135/228 residues (FIG. 5). The inventors built those mutations back into VLPs to validate them as escape mutations. Indeed, the G135D mutation in the Japan/305+ HA protein context eliminated inhibition by mAb 8M2, and the T193K mutation reduced inhibition (Table 12). The T193K mutation eliminated inhibition by mAb 8F8, and R137Q markedly reduced inhibition for this antibody only. The K156E mutation conferred escape only for 2G1.

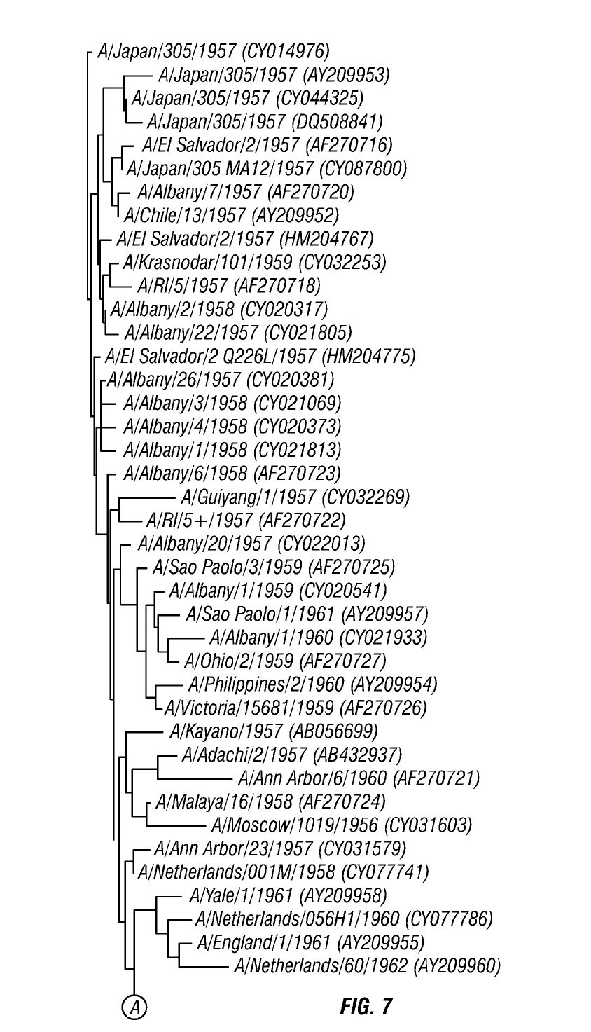

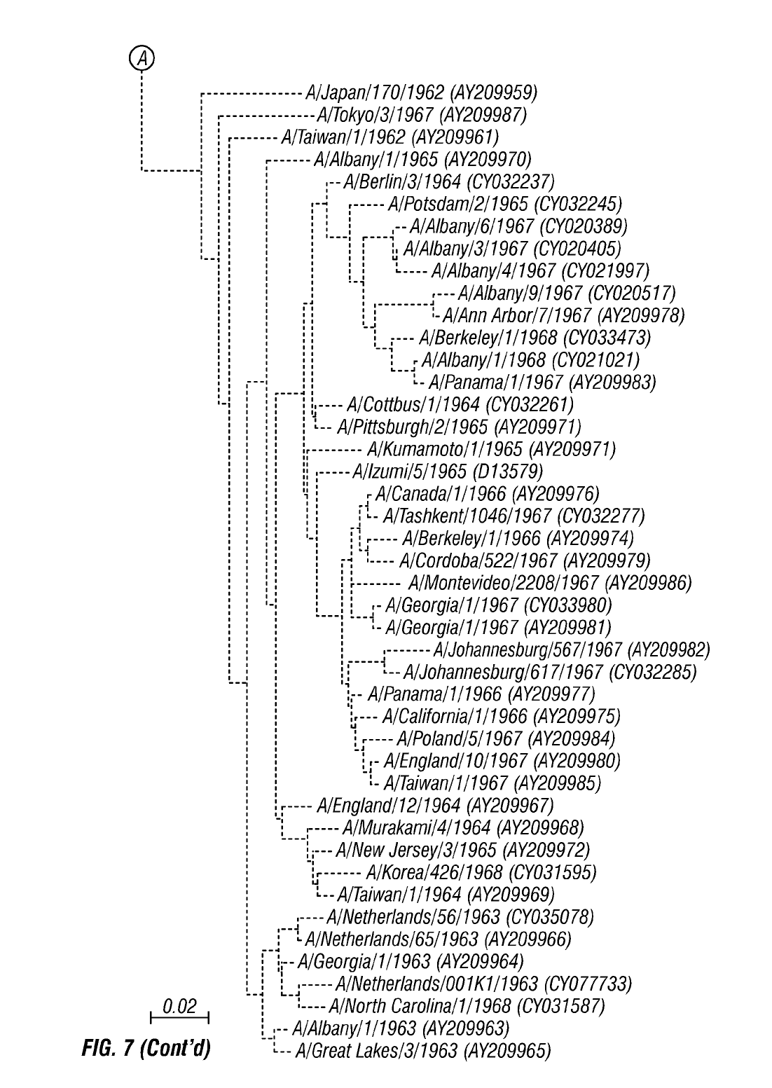

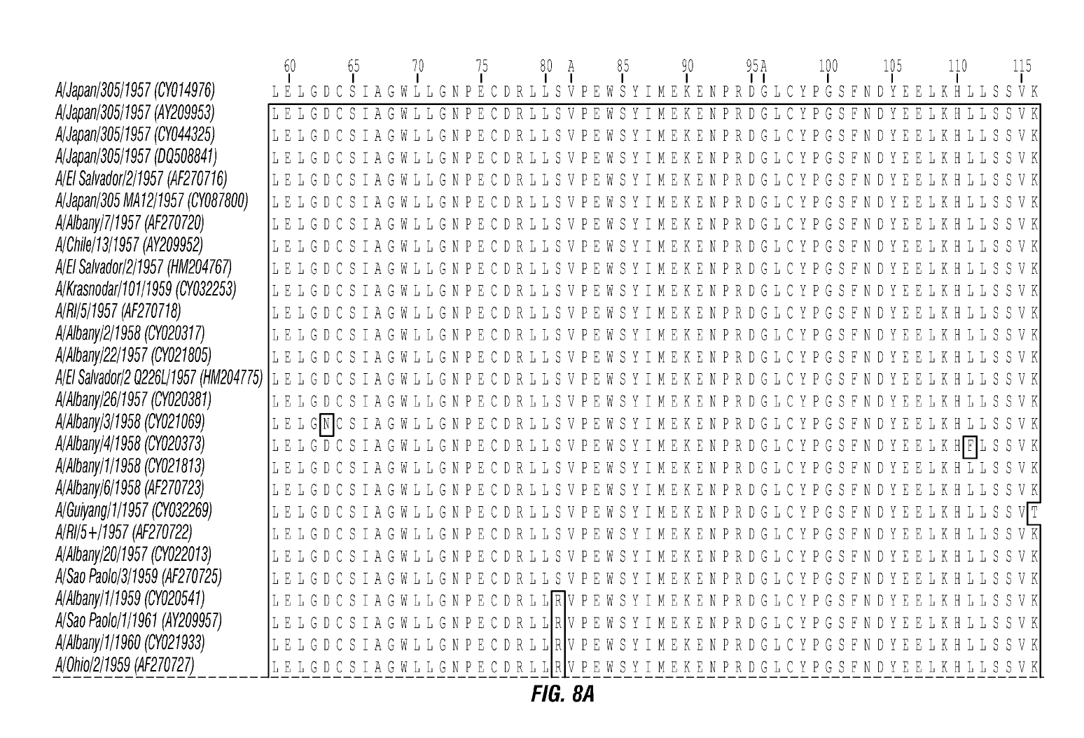

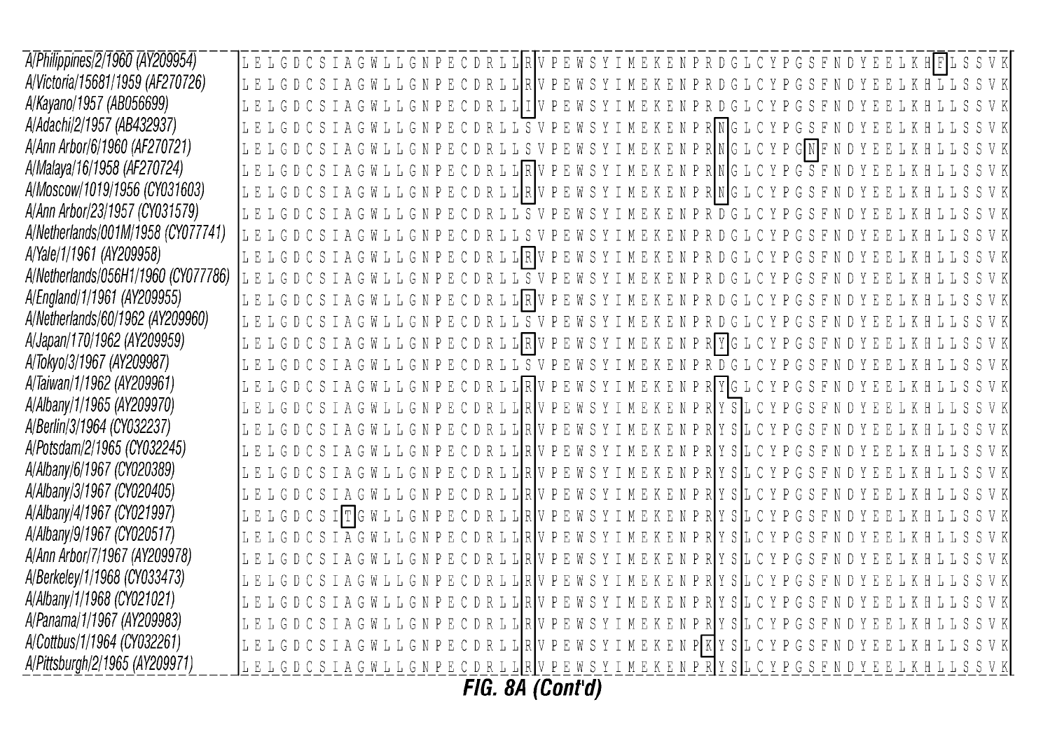

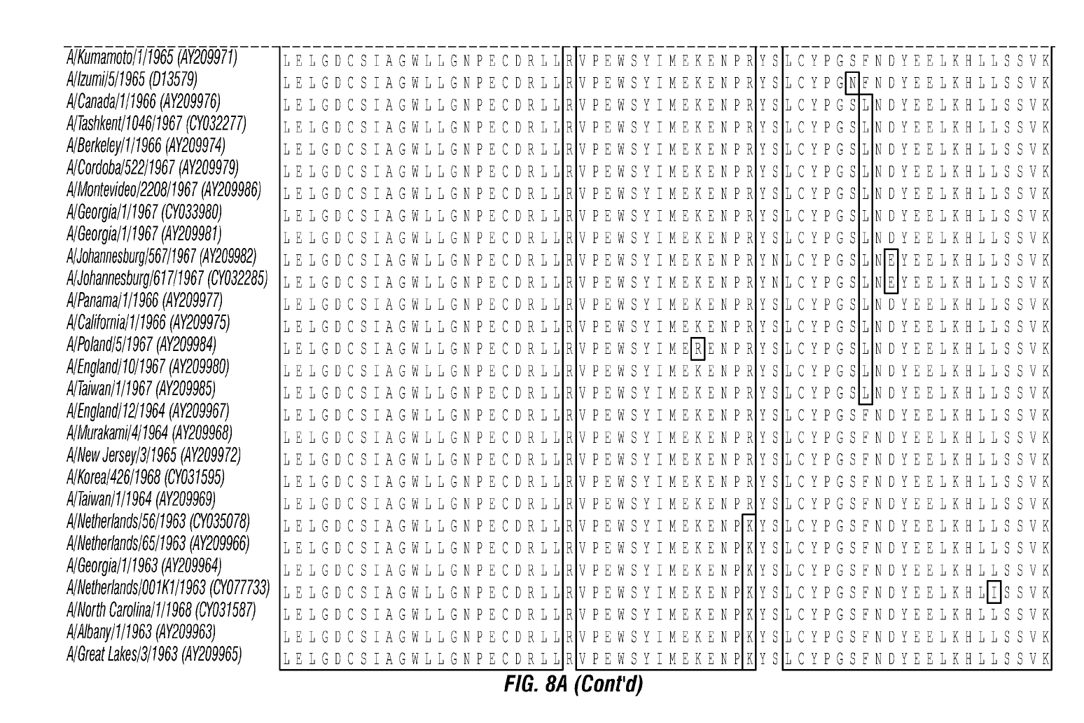

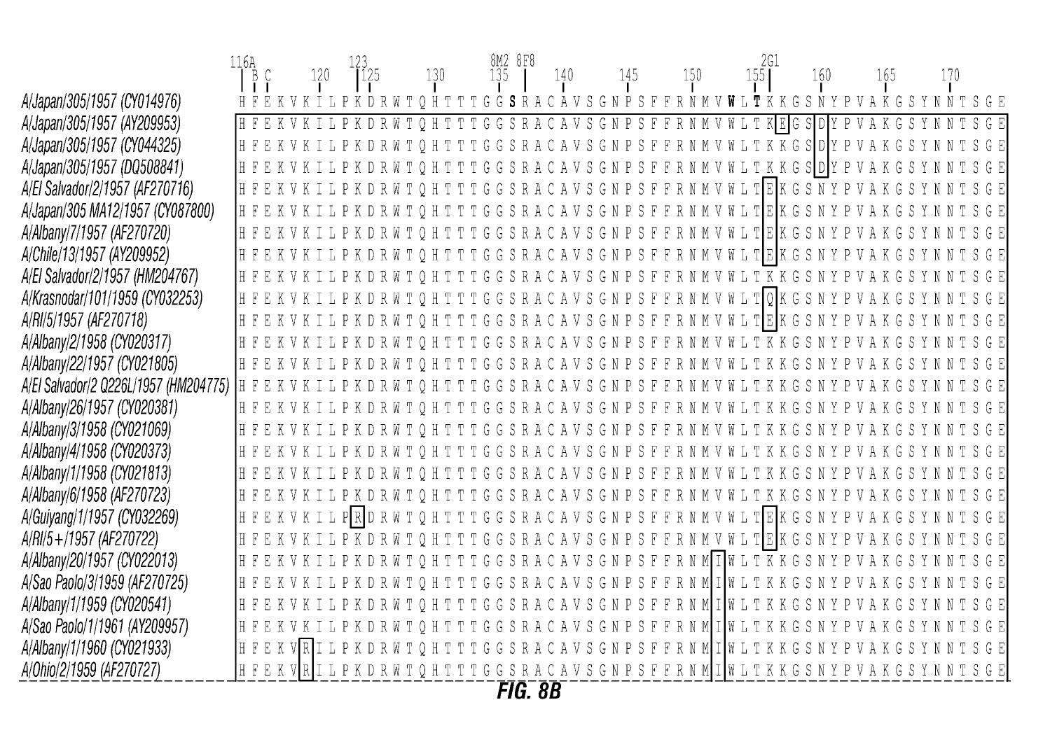

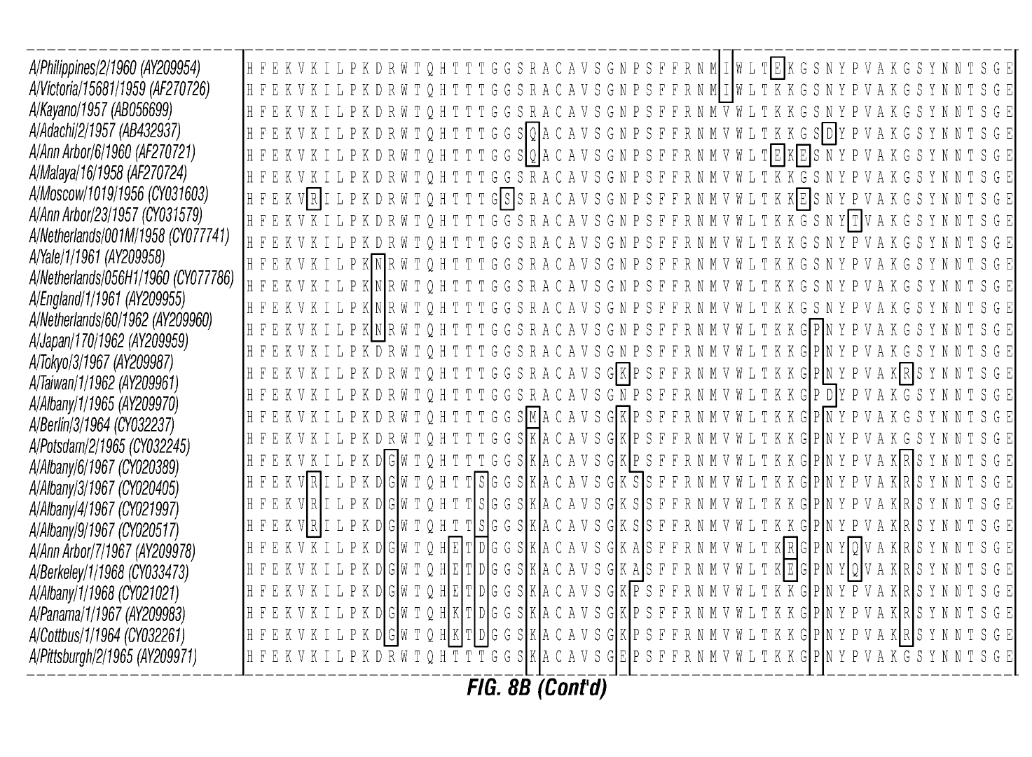

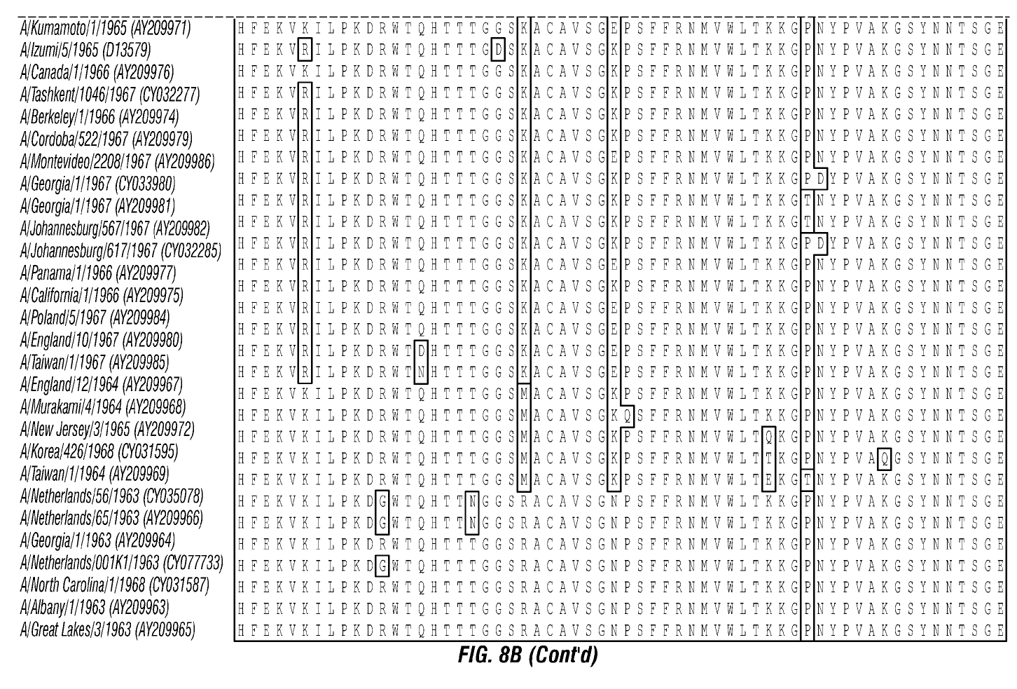

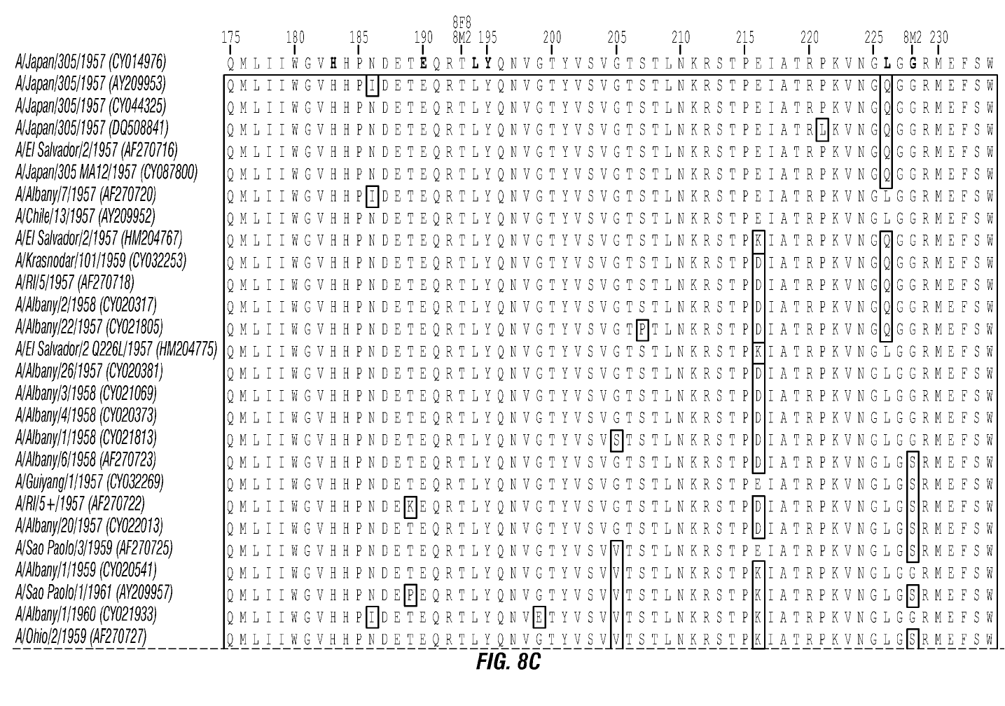

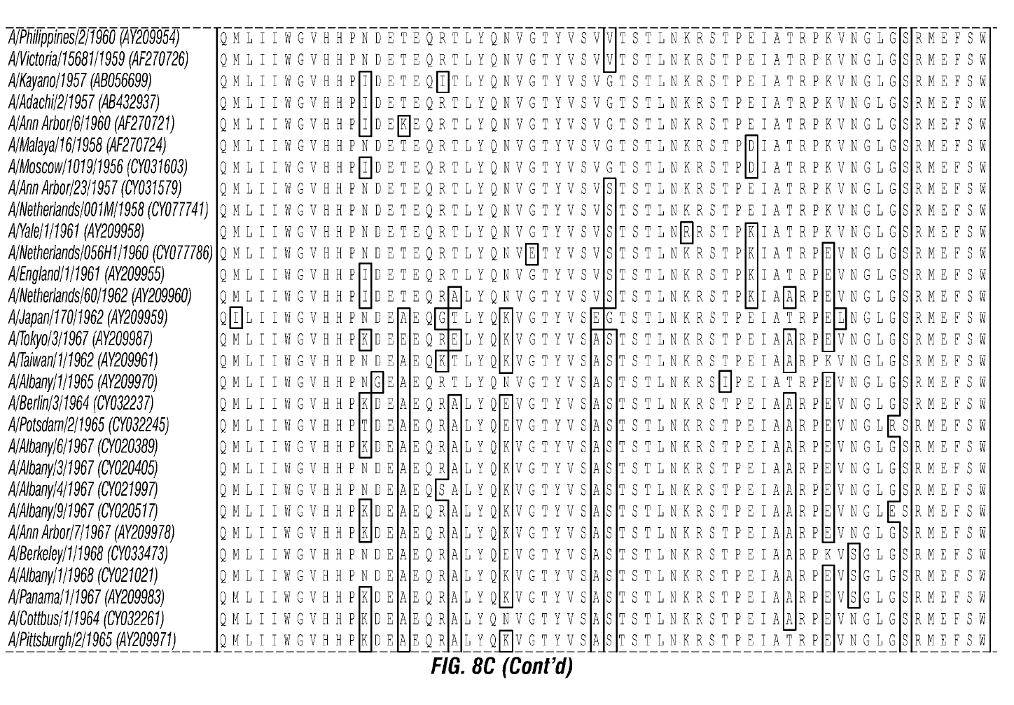

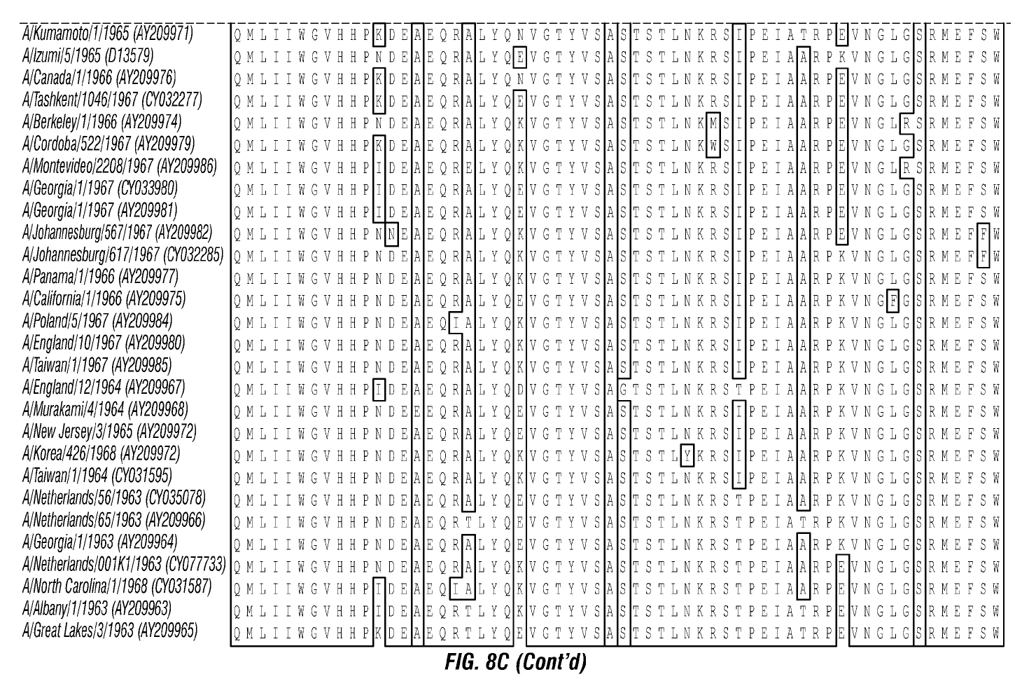

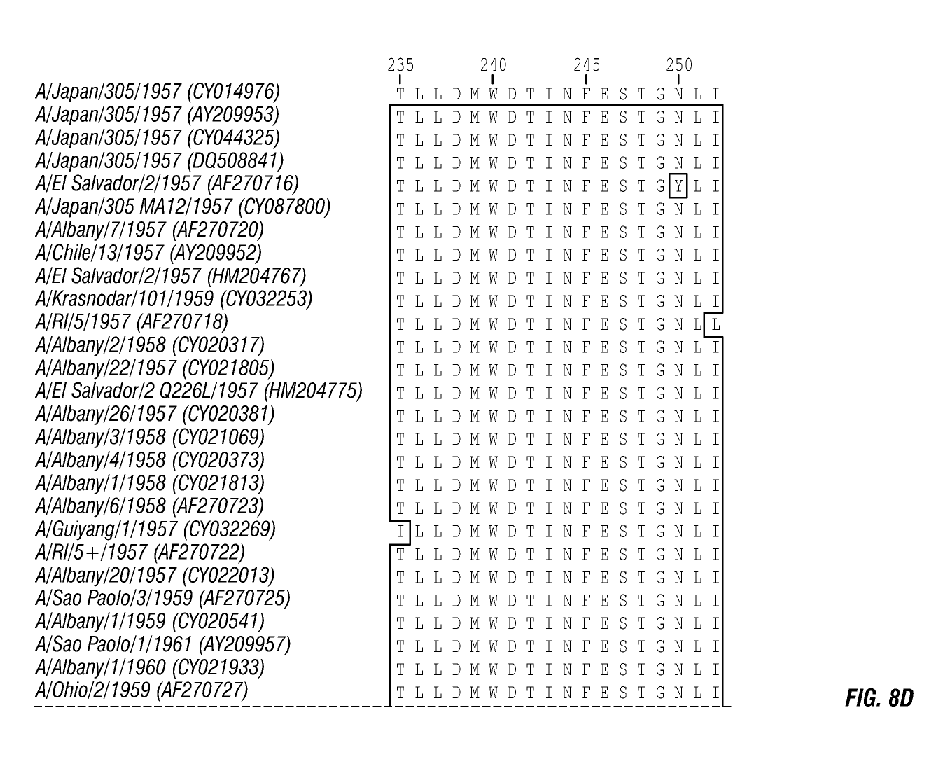

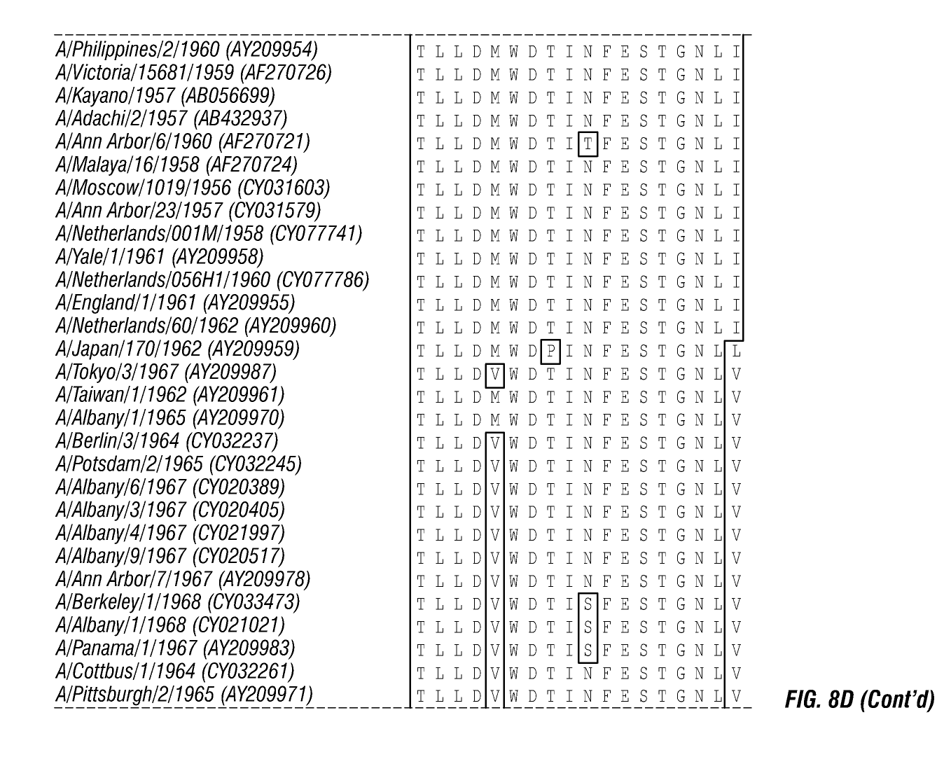

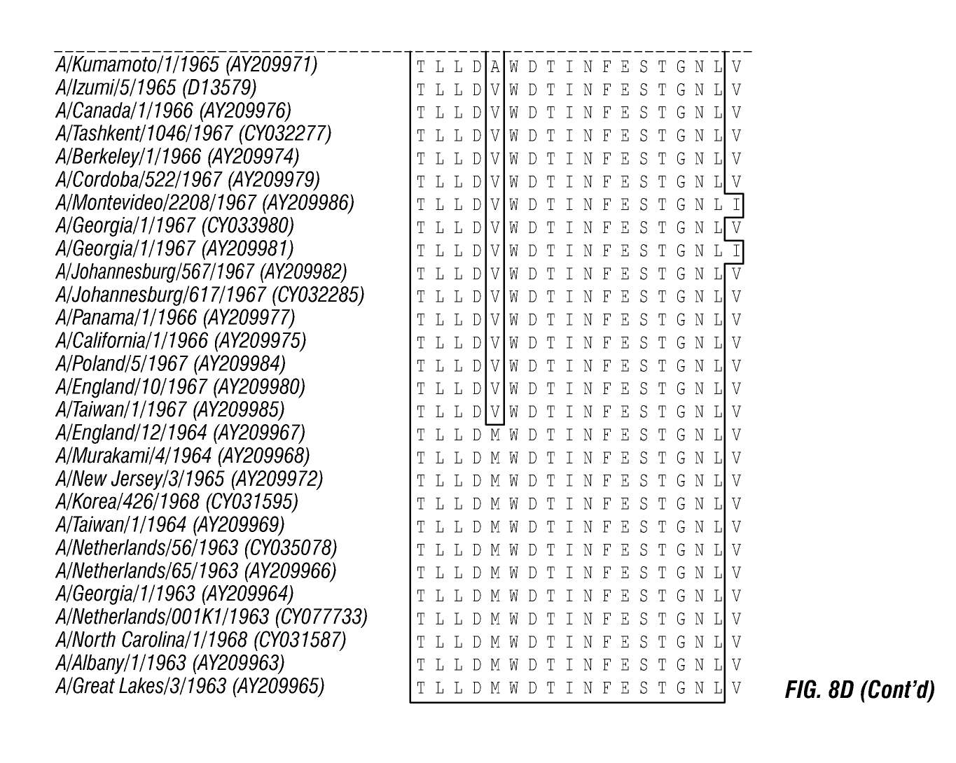

Sequence analysis of H2N2 HA. The inventors next sought to understand the influence of naturally-occurring H2N2 HA antigenic drift mutations on the activity of their antibodies using previously isolated field strains of H2 viruses. A phylogram of the amino acid sequence of all naturally-occurring, non-redundant human H2N2 HAs revealed two distinct populations of early (1957-1960) and late (1963-1968) H2N2 influenza strains (FIG. 7). The inventors performed a multiple sequence alignment of those strains in the order of the phylogram to document the sequence variability, particularly in the key residues of the escape mutations (FIG. 8). Residue G135 was well-conserved except for aspartic acid in A/Kumamoto/1/1965 and serine in A/Moscow/1019/1965 (FIG. 8); it is questionable whether the latter is truly a 1965 strain as it is very similar to the 1957-1961 strains. The arginine in position 137 of H2 HA has mutated to a glutamine in two early H2 strains (so the above R137Q escape mutant is present in naturally-occurring H2N2 viruses), to a methionine in six late H2 strains, and a lysine in most other late H2N2 strains (FIG. 8). A T193A mutation is found in almost all late H2N2 strains, although two H2 HAs display a glutamic acid in this position. A K156E mutation is found in occasional early or late H2 strains; glutamine or threonine also were found in this position, but the original lysine predominated overall. Interestingly, the lysine residue at position 156 on HA that is critical for recognition by the heterosubtypic 2G1 antibody is present in the HA of every pandemic virus isolated to date (1918 H1, 1957 H2, 1968 H3, 2009 H1) and even the HA of the H5N1 A/VietNam/1203/2004 strain that is highly virulent in humans.

Residue 228 is split between the serine typical of human receptor-specificity and the glycine of avian receptor-specificity, although only a serine is found in this position in later strains. It should be noted that human H2N2 viruses have been passaged many times in eggs and that the passage history for some of the strains is unknown; whether viruses with avian-receptor specificity truly co-circulated in 1957 or whether those isolates are drift variants from subsequent egg passage is controversial (Pappas et al., 2010; Connor et al., 1994; Matrosovich et al., 2000). The inventors introduced some of these mutations into the A/Japan/305+/1957 background to test the specificity of their H2N2 antibodies in HAI assays against VLPs. MAb 8F8 was very sensitive to changes in position 137, with an R137M mutation leading to loss of inhibition and an R137K mutation to a marked reduction of inhibition. This finding might explain why mAb 8F8 does not inhibit late H2N2 strains, although changes at other residues not identified by escape mutations might contribute.

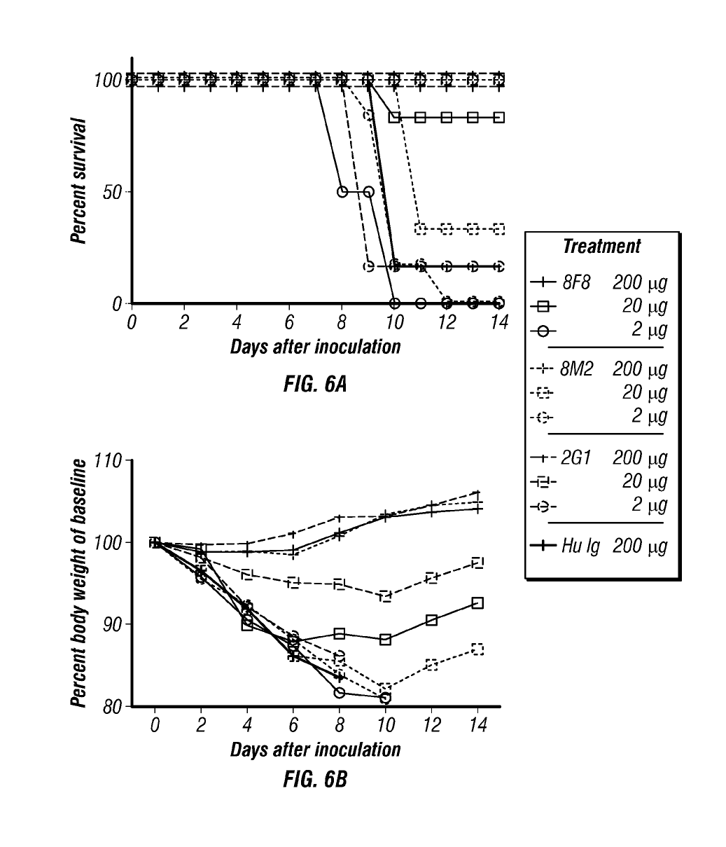

In vivo therapeutic efficacy of H2 antibodies. The inventors next tested the H2-reactive mAbs in a therapeutic mouse model of H2N2 influenza infection (FIGS. 6A-B, Table 13). MAbs 8F8, 8M2, and 2G1 each protected all animals at the highest dose level of 200 μg; only mAb 2G1 also protected all animals at the intermediate dose level and a single animal at the lowest dose level (FIG. 6A). This trend was reflected in the animal weight curves, with animals in the high-dose groups gaining weight by day 14 as compared to baseline (FIG. 6B). At the highest dose level, the antibodies were able to reduce H2N2 lung titers between 2.6 log₁₀ PFU/mL (for 2G1) and 2.2 log₁₀ PFU/mL (for 8M2) when compared to the IgG control (Table 13).

TABLE 13
Therapeutic efficacy of H2N2-reactive mAbs against virus replication in
mice inoculated with A/Albany/6/1958 H2N2 virus. Four mice were
inoculated intranasally with 5 x LD₅₀ and administered mAb
8F8, 8M2, 2G1, or human IgG i.p. 24 h later. Mice were euthanized
on day 4 after inoculation for the determination of lung titers

| | Dose | Mean lung virus titer |
|---|---|---|
| Antibody | [μg/mouse] | [log₁₀ PFU/mL ± SD] |
| 8F8 | 200 | 4.4 ± 0.1* |
| | 20 | 5.8 ± 0.1* |
| | 2 | 6.5 ± 0.2* |
| 8M2 | 200 | 4.7 ± 0.3* |
| | 20 | 6.3 ± 0.2* |
| | 2 | 6.8 ± 0.1 |
| 2G1 | 200 | 4.3 ± 0.3* |
| | 20 | 5.0 ± 0.5* |
| | 2 | 6.3 ± 0.4* |
| IgG control | 200 | 6.9 ± 0.1 |

*At the α = 0.025 level controlling the overall type I error at 7.5%, the lung homogenates differ from the IgG control group by the Wilcoxon rank sum test (p < 0.05).

Reactivity of H3N2 antibodies. Surprisingly, the H2-reactive mAb 2G1 also inhibited the pandemic 1968 H3 virus, but not an H3 virus from 1981 (Table 11). The H3-specific antibodies 7A13 and 11J19 also inhibited A/Hong Kong/1/1968 H3N2, but not this later strain from 1981 (Table 11), suggesting that these antibodies do not display significant cross-reactivity within this influenza subtype. The previously published murine hybridoma derived mAb S139/1 did show potent HAI activity against A/Hong Kong/1/1968 (H3N2), consistent with previous experiments (Yoshida et al., 2009) (Table 11). However, mAb S139/1 did not inhibit A/Singapore/1/57 (H2N2) even at an antibody concentration of 50 μg/mL. Although the inventors did not test mAb S139/1 against A/WSN RG/33 (H1N1), a strain shown by Yoshida et al. to be inhibited, mAb S139/1 did not inhibit A/California/04/2009 (Table 11) or any of eight other H1N1 strains that were tested, even at 50 μg/mL (data not shown).

### C. Discussion

B cells specifying mAbs to 20^{th} century pandemic influenza viruses can still be detected in the peripheral blood of humans. The persistence of virus-specific memory B cells in the circulation is remarkable. The inventors previously showed that H1-specific human mAbs to the 1918 H1N1 pandemic virus can be cloned from the peripheral blood of survivors of the pandemic many decades after circulation of that virus (Yu et al., 2008a). Here, the inventors used a similar approach (Yu et al., 2008b) to clone neutralizing human mAbs against 1957 H2N2 or 1968 H3N2 pandemic viruses. Influenza antibodies also can be cloned from plasmablasts of recent vaccinees or those convalescing from disease (Wrammert et al., 2008), but this technology is not suitable for a pathogen that is no longer in circulation and for which a routine vaccine is unavailable. In contrast, human hybridoma technology can be used to generate mAbs against an antigen that has not been in human circulation for at least 43 years, such as H2N2 influenza. In summary, the inventors have shown for all three influenza pandemics of the 20^{th} century (1918, 1957, and 1968) that B cells specific for the pandemic virus can still be found in the peripheral blood of human beings in the 21^{st} century.

Antibodies targeting the H2 RBD may have been present on a population level. The first major epitope mapping of H2N2 HA was performed almost 30 years ago using murine mAbs (Yamada et al., 1984). Interestingly, half of these antibodies—like mAb 8M2—did not inhibit virus with avian receptor-specificity; this phenomenon was even highlighted by Yamada et al. in the title of their manuscript (Yamada et al., 1984). Receptor-specificity can influence antibody inhibition when the epitope includes residues that mediate that receptor-specificity (i.e., that are part of the RBD). Unlike H1N1 HA in early epitope mapping studies (Caton et al., 1982), H2N2 was not found to have discrete murine epitopes, but to have overlapping epitopes on its globular head, as the antibodies isolated competed with each other for binding to HA over the RBD (Yamada et al., 1984). These findings are consistent in principle with the epitope mapping of human mAbs in this study that elicited escape mutations immediately adjacent to the RBD. The number of human antibodies in this study is limited, but taken together with the prior work by Yamada et al. (1984), suggests that most of the circulating B cells specific for H2N2 influenza are targeted to the RBD. Why both the human and the murine immune responses to H2 are so focused on the relatively conserved RBD is unclear. Understanding this phenomenon better might help to improve current influenza vaccines.

Antibodies that contact amino acid residues that are components of the RBD have been described previously (Knossow and Skehel, 2006) including an H1N1-specific antibody that essentially imitates sialic acid by reaching into the RBD (Whittle et al., 2011). Although the inventors' H2N2 antibodies likely make contact deeper within the RBD pocket, such contact residues are difficult to identify by the escape mutation method the inventors used since such viruses likely would be reduced in replicative capacity. The antibody CDR-H3 loop is typically longer than the other five complementarity determining region (CDR) loops and would be a prime candidate for insertion into the RBD. Since antibody repertoires of humans contain more antibodies that have longer CDR-H3 loops than repertoires of mice (Wu et al., 1993), human mAbs might be expected to be even better suited to reach into the recessed RBD. This discrepancy between the common features of naturally-occurring human antibodies and experimentally induced murine mAbs is why it is important to study the human antibody response.

Stem antibodies to H2 HA were unlikely to be present on a population level. Human antibodies specified by the V_{H}1-69 germ line variable gene segment have been cloned and neutralize both H1 and H5 strains by binding to the HA stem region (Ekiert et al., 2009; Corti et al., 2010; Wrammert et al., 2011; Sui et al., 2009). Interestingly, these mAbs also neutralize avian H2 strains and bind to the HA of human H2 strains, but do not neutralize those (Throsby et al., 2008). In contrast, the mouse antibody C179 neutralizes human H2 virus (Okuno et al., 1993). Recently, a stem antibody encoded by the V_{H}3-30 germline gene segment was shown to neutralize H1N1 and H3N2 viruses, but no neutralization data on H2N2 viruses were presented (Corti et al., 2011).

There is historic evidence that adults were relatively protected against disease with H2N2 infection because of the occurrence of heterosubtypic immunity induced by prior heterologous infection (Epstein, 2006). This protection may have been conferred by antibodies, but also could have been mediated by cross-reactive cellular immunity. Given that stem region neutralizing antibodies are rare (Corti et al., 2010) and that no human stem antibody has been shown to conclusively neutralize human H2N2 virus, the inventors hypothesize that stem region antibodies were not the principal cause for the limited duration of circulation of H2N2 in humans. Thus, it seems more likely that the H2N2 virus was limited in its circulation because the HA did not achieve sufficient structural variation by drift to protect the RBD from recognition by RBD-specific antibodies, for example by acquiring glycosylation sites in domains adjacent to the RBD.

2G1-like antibodies may have provided relative protection from H3 virus. Neither the H2-specific mAbs 8M2 or 8F8, nor the H3-specific mAbs 7A13 or 11J19 neutralized the other subtype, respectively. Also, the inventors were unable to confirm that the previously described murine mAb S139/1 is a cross-neutralizing globular head antibody, although it did inhibit 1968 H3 virus, as described (Yoshida et al., 2009). Even though the inventors were not able to test mAb S139/1 against all the strains presented in the earlier paper, its breadth seemed limited in their hands. However, the human H2 antibody 2G1 did inhibit the 1968 H3 influenza virus, which is a very unusual phenotype that has not been described previously. It is likely that H2 virus was the inciting event for mAb 2G1, since infection with pandemic viruses is near universal and H2 virus circulated before H3, and since this antibody exhibits more potent inhibition of H2 virus. The relative conservation of the critical residue 156 may have been the structural basis for this cross-reactivity, though other residues likely contributed. Heterosubtypic HA globular head domain specific antibodies are probably rare because of the variability in the dominant antigenic loops, but the presence of 2G1-like antibodies might explain the diminished severity of the 1968 H3N2 pandemic as opposed to the 1957 H2N2 pandemic—a phenomenon that had previously been attributed to cross-reactive N2 neuraminidase antibodies (Wright et al., 2007).

H2 mAbs might be useful for diagnostic or therapeutic purposes. Experimental H2N2 vaccine candidates exist, but may not be protective after a single dose (Hehme et al., 2002). Therefore, passive transfer of antibodies such as mAb 8F8, 8M2, or 2G1 could be used in case of a 1957-like virus pandemic to protect high-risk individuals. Also, these antibodies could be useful as diagnostic reagents, or to differentiate H2N2 viruses with human or avian receptor-specificity.

### Example 4

### A. Methods

Hybridoma generation and recombinant antibody expression. Peripheral blood mononuclear cells were isolated from a 47 year old healthy human subject, EBV-transformed in 384 well plates (Nunc) in the presence of 2.5 μg/mL CpG ODN 2006 (Invivogen), 10 μM Chk2 inhibitor II (Sigma C3742), and 1 μg/mL cyclosporine A (Sigma), essentially as previously described (Yu et al., 2008a; Yu et al., 2008b). Supernatant was screened by ELISA against a panel of recombinant soluble HA proteins. B-cells were fused with HMMA2.5 myeloma cells, cultured in selection medium, and cloned by limiting dilution. The antibody genes were cloned molecularly from mRNA isolated from the cloned hybridoma cell line using previously described primer sets (Smith et al., 2009) into pGEM-T Easy vector (Promega) and eventually into pEE12.4/pEE6.4 mammalian expression vectors (Lonza), from which they were expressed (Xu et al., 2009b) and FPLC-purified on a protein G column (for IgG₁) or via CaptureSelect λ resin (for Fab; BAC B.V.). Analysis with the international ImMunoGeneTics information system (IMGT) (Lefranc, 2003) identified mAb 5J8 as an antibody encoded by the IGHV4-b*01, J4*02, D3-3*02; IGLV3-21*02 or *03, J2*01 or J3*01 variable gene segments. Recombinant antibody was used for all following studies.

Virus-like particle (VLP) expression and hemagglutination inhibition (HAI) assays. Expression plasmids encoding HA protein molecules were co-expressed with neuraminidase to produce VLPs in 293T cells (Chen et al., 2007; Yu et al., 2008a). HAI assays were performed as described (World Health Organization Collaborating Centers for Reference and Research on Influenza) using VLPs (for 1918 influenza) or live virus (for all other strains). MAb 5J8 inhibited all tested H1N1 influenza strains from 1918 until 1977 and the pandemic 2009 virus, but not the seasonal H1N1 strains from 1999 or 2007 (Table 14). HAI activity was most potent against 1918 VLPs or 1930 virus at 40 ng/mL.

TABLE 14
Hemagglutination inhibition (HAI) activity of 5J8 in representative 20^{th}
century influenza H1N1 strains and sequence variation in key residues of those strains

| | HAI | Neut | | | | |
|---|---|---|---|---|---|---|
| | activity | titer | HA residue (based on H3 numbering) | | | |
| H1N1 strain | μg/mL | μg/mL | 133A | 137 | 199 | 222 |
| A/South Carolina/1/1918 | 0.04 | NT | K | A | D | K |
| A/swine/Iowa/15/1930 | 0.04 | <0.025 | R | A | D | K |
| A/Weiss/1943 | 5 | 10 | R | A | N | K |
| A/L3/1947 | 0.08 | 0.1 | R | A | N | K |
| A/New Jersey/11/1976 | 0.32 | NT | R | A | D | K |
| A/USSR/92/1977 | 0.63 | 0.2 | R | A | N | K |
| A/New Caledonia/20/1999 | >20 | >20 | Del | A | N | K |
| A/Brisbane/59/2007 | >20 | NT | Del | A | N | K |
| A/California/04/2009 | 0.09 | 0.0017 | K | A | D | K |

| | | | | |
|---|---|---|---|---|
| Amino acid point mutations | Del: 1665 | A: 1861 | N: 1811 | K: 1813 |
| in naturally occurring | K: 137 | T: 11 | D: 19 | E: 2 |
| H1N1 field strains† | R: 45 | V: 6 | S: 4 | P: 1 |
| | T: 31 | S: 2 | H: 1 | Del: 1 |
| | X: 2 | X: 1 | I: 1 | |
| | | Del: 1 | K: 1 | |
| | | | Del: 1 | |

Escape mutations

| | | | | |
|---|---|---|---|---|
| A/swine/Iowa/15/1930 | | A->T | | |
| A/L3/47 | R->I | | | K->Q |
| A/California/04/2009 | K->Q | | D->H | |

†All human H1N1 HA protein sequences were acquired from Influenza Research Database from 1918-2008 - search performed on Mar. 26, 2011;
sequences that were duplicates or from viruses generated through genetic manipulation were removed prior to analysis.
NT = not tested.

Microneutralization assay. Different dilutions of antibody were incubated with 5 log₁₀ TCID₅₀ of each virus for 1 hour. The mixture was used to infect MDCK cells in triplicate for an hour at 37 degrees. The plate was harvested 3 days later and read in an HA assay. The endpoint was the lowest concentration that gave no HA activity. The microneutralization assay generally agreed well with the HA assay (Table 14)

Biosensor studies to determine affinity. Binding affinities of 5J8 Fab to recombinant trimeric His-tagged HA proteins were measured using anti-Penta-HIS tips on an Octet Red instrument (FortéBio); 1918 HA variants were created with the QuikChange II XL mutagenesis kit (Agilent). The extracellular domain of full length 1918 HA cDNA was cloned into a pcDNA3.1(+) construct that contained an SGR linker, a thrombin recognition site, a fibritin trimerization domain, and a 6× His tag. The protein was expressed in 293F cells and purified from the supernatant on an ÅKTA FPLC using Ni²⁺ columns (GE). Binding affinity of 5J8 Fab to 2009 H1 HA protein was 2.6×10⁻⁸ M (Table 15).

TABLE 15
Binding of Fab 5J8 to soluble influenza HA proteins by biolayer
interferometry

| SC/18 mutant | | | |
|---|---|---|---|
| or Ca/04 | K_{D} [M] | kₒₙ [1/Ms] | k_{off} [1/s] |
| SC/18 | 1.9 × 10⁻⁷ | 2.0 × 10⁵ | 3.6 × 10⁻² |
| wt | | | |
| SC/18 | ND | ND | ND |
| del133A | | | |
| SC/18 | ND | ND | ND |
| 133A K->I | | | |
| SC/18 | ND | ND | ND |
| 133A K->T | | | |
| SC/18 | ND | ND | ND |
| A137T | | | |
| SC/18 | 1.7 × 10⁻⁷ | 1.9 × 10⁵ | 3.1 × 10⁻² |
| D199H | | | |
| SC/18 | ND | ND | ND |
| K222Q | | | |
| Ca/04 | 2.6 × 10⁻⁸ | 2.5 × 10⁵ | 6.5 × 10⁻³ |
| wt | | | |

SC/18 = A/South Carolina/1/1918;
Ca/04 = A/California/04/2009;
ND = not detectable

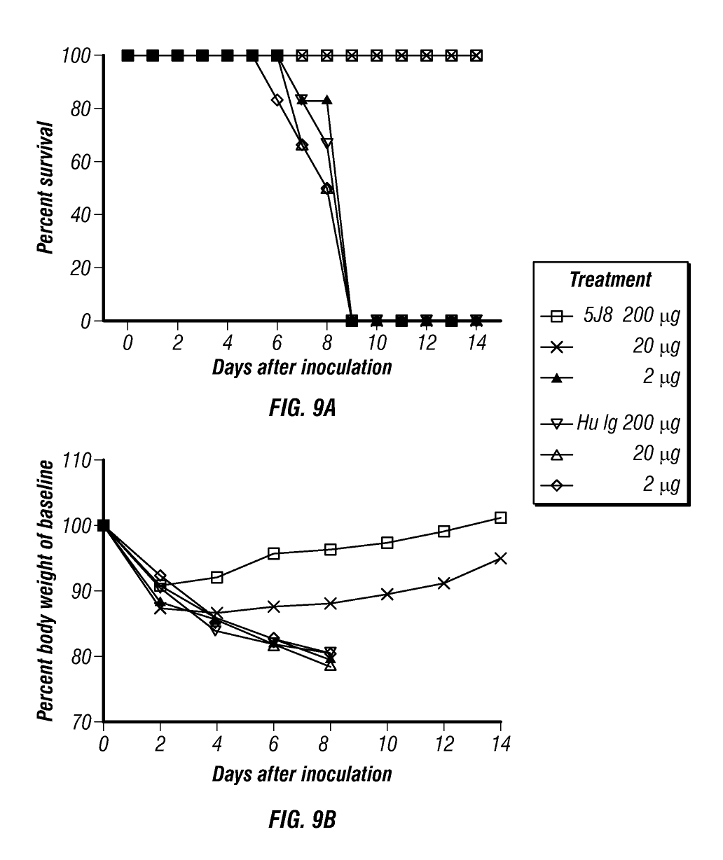

Animal studies. The inventors tested 5J8 for therapeutic efficacy in a lethal animal model of 1918 virus infection (Yu et al., 2008a). Female BALB/c (8-week-old, weighing approximately 20 grams) mice were inoculated intranasally with five times the 50% lethal dose (LD₅₀) in a 50 μL volume of the virulent reconstituted virus. At 24 h after inoculation, mice were administered 200, 20, or 2 μg of 5J8 or an equal amount of human IgG (Sigma) each by the intraperitoneal route, in groups of ten mice. Mice were observed for weight loss for 14 days (FIG. 9B). Subsets of four animals treated with the mAbs were euthanized on day four after infection, and whole lungs were homogenized in one mL of sterile PBS. Virus titer in lung tissue homogenates was determined by plaque titration in MDCK cell monolayer cultures. MAb 5J8 protected all animals at the high and medium dose from lethal challenge (FIG. 9A) and reduced lung virus titers as compared to IgG control by 2.6 log₁₀ PFU/mL at the 200 μg dose level, by 2.0 log₁₀ PFU/mL at the 20 μg dose level, and by 0.7 log₁₀ PFU/mL at the 2 μg dose level (Table 16).

TABLE 16
Therapeutic efficacy of mAb 5J8 against virus replication in
mice inoculated with 1918 influenza A virus

| | Dose | Mean lung virus titer |
|---|---|---|
| Antibody | (μg/mouse) | (log₁₀ PFU/mL ± SD)* |
| mAb 5J8 | 200 | 4.1 ± 0.2 |
| | 20 | 4.9 ± 0.7 |
| | 2 | 5.9 ± 0.1 |
| Human IgG control | 200 | 6.7 ± 0.1 |
| | 20 | 6.9 ± 0.0 |
| | 2 | 6.6 ± 0.4 |

*At the α = 0.025 level controlling the overall type I error at 7.5%, the lung homogenates of the mAb 5J8 groups had lower virus titers than the human IgG control groups for all dose levels by the Wilcoxon signed-rank test (p = 0.01429 for the high-dose groups, p = 0.01429 for the intermediate-dose groups, p = 0.0147 for the low-dose groups).

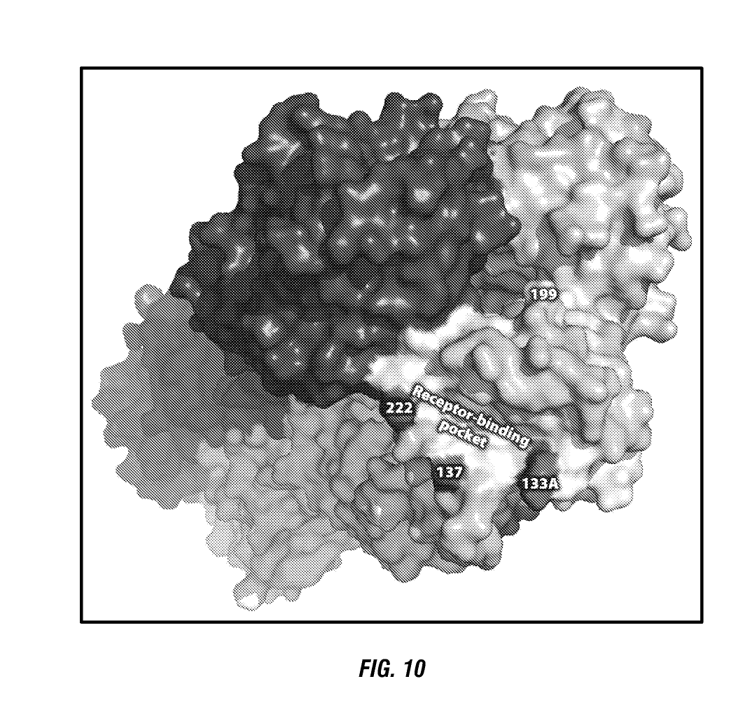

Isolation and characterization of antibody escape mutant viruses. The inventors selected and sequenced the HA gene of new antibody escape mutant viruses (Caton et al., 1982; Yewdell et al., 1979). Following mAb 5J8 selection, HA mutations were identified in positions 133A, 137, 199, or 222 (based on H3 numbering (Stevens et al., 2004)) in some of these strains (Table 14). The inventors then introduced these naturally-occurring mutations into the soluble 1918 HA protein for in vitro binding studies to validate the effect of those putative escape mutations. 133A K→I, A137T, or K222Q each eliminated binding of mAb 5J8 to the mutant protein (Table 15). However, binding of mAb 5J8 to the D199H mutant (that the inventors only detected in concert with a 133A K→Q mutant) was retained (Table 15). The inventors were technically unable to create the 133A K→Q mutant construct, but this residue likely would mediate escape based on the effect of the validated 133A K→I escape mutant. Of note, all of these escape mutations are outside of conventionally-defined antigenic sites (Brownlee and Fodor, 2001; Caton et al., 1982) and are located between the receptor-binding pocket and the Ca₂ antigenic site (FIG. 10).

Review of H1N1 sequences. The inventors then queried the Influenza Research Database (IRD; world-wide-web at fludb.org) for all naturally-occurring, non-redundant human H1N1 viruses between 1918 and 2008 to identify the frequency of sequence variation in the residues mediating escape from mAb 5J8. Of note, residues 137 (alanine) and 222 (lysine) were each found in greater than 98% of all the sequences (Table 14). There was greater sequence variability at the position of the critical residue 133A: The lysine from the 1918 virus was replaced by arginine (a conservative mutation to a similarly positively-charged residue) as early as in the 1930 virus without a subsequent change in HAI activity. A lysine was seen again in the 2009 H1N1 virus, but residue 133A was deleted in previous seasonal strains. When built back into 1918 HA, this deletion by itself does lead to loss of binding (Table 15). In fact, over 80% of all strains in the dataset showed this deletion, but this high percentage is biased because most sequences in the IRD are recent. The 133A deletion has been observed rarely prior to 1997, but has been dominant since.

Thus, MAb 5J8 recognizes a novel, conserved epitope on the globular head of H1N1 HA that is characterized by residues 133A, 137, and 222 in close proximity to the RBD, which illuminates the molecular basis for breadth of neutralization of mAb 5J8. There likely are very strong structural constraints on this epitope. It is certainly possible that mAb 5J8 makes contacts deeper within this pocket that H1N1 cannot change without loss of replicative capacity and that would not be revealed by escape mutations.

In conclusion, mAb 5J8 helps paint a more detailed picture of why older subjects possessed 2009 H1N1 cross-reactive HA antibodies prior to exposure to the 2009 H1N1 virus (World Health Organization Collaborating Centers for Reference and Research on Influenza), since epitopes other than the Sa site (Krause et al., 2010; Manicassamy et al., 2010; Wei et al., 2010; Xu et al., 2010b) or stem epitopes (Corti et al., 2010; Ekiert et al., 2009; Sui et al., 2009; Throsby et al., 2008; Wrammert et al., 2011) contribute to this cross-protective effect. The relative importance of these epitopes for the antiviral humoral response may vary from person to person. The fact that it took decades for H1N1 viruses to escape mAb 5J8-like antibodies may reflect the fact that such antibodies are relatively rare and thus pose little evolutionary pressure on H1N1. The inventors propose that presenting the RBD epitope in a more exposed way without surrounding hypervariable loops might make it more immunogenic and thus might contribute to a universal influenza vaccine design strategy.

### Example 5

### A. Materials and Methods

Hybridoma generation and recombinant antibody expression. Acquisition of human blood samples was approved by the Vanderbilt University Institutional Review Board. The animal studies were approved by the Institutional Review Boards of the CDC. PBMCs were isolated from a 47-year old healthy female donor with Histopaque-1077 (Sigma), EBV-transformed in 384 well plates (Nunc) in the presence of 2.5 μg/mL CpG ODN 2006 (Invivogen), 10 μM of Chk2 inhibitor II (Sigma C3742), and 1 μg/mL cyclosporine A (Sigma), essentially as described previously (Yu et al., 2008a; Yu et al., 2008b). Supernatants from wells containing EBV-transformed lymphoblastoid cell lines were screened for binding activity by ELISA against a panel of recombinant soluble HA proteins. Positive wells were fused with HMMA2.5 myeloma cells and cloned molecularly using previously described primer sets (Smith et al., 2009) into pGEM-T Easy vector (Promega) and eventually into pEE12.4/pEE6.4 mammalian expression vectors (Lonza) from where they were expressed (Xu et al., 2010b) and purified on a protein G column using an ÅKTA chromatography instrument (GE). All following studies were performed using recombinant Abs. The inventors used Kabat numbering as determined using the Abnum server (Abhinandan and Martin, 2008) for the antibodies and an H3 numbering scheme (Stevens et al., 2004) for HA. Antibody clonality was defined strictly by shared V_{H} gene, shared VDJ junction and a sequence of shared somatic mutations.

Generation and purification of recombinant soluble HA molecules. 1918 or 2009 influenza HA constructs were ordered sequence-optimized for expression in human cells (GenScript) based on the extracellular domain of the respective HA, a thrombin recognition cleavage site, a fibritin trimerization domain, and a 6× His-tag (Stevens et al., 2004). The constructs were cloned into pcDNA3.1(+) (Invitrogen), expressed in 293F cells (Invitrogen), purified over nickel columns using an ÅKTA chromatography instrument (GE), and concentrated with Amicon Ultra centrifugal filters with a 30 kD molecular weight cut-off (Millipore).

ELISA. 384-well clear plates (Nunc 242757) were coated with HA at 1 μg/mL in D-PBS overnight, blocked with 0.5% cow milk, 0.2% goat serum, and 0.05% TWEEN 20 (Sigma) in D-PBS. Five μL of hybridoma supernatant per well were transferred to 25 μL of blocking solution with a multi-channel pipette. Secondary goat anti-human IgG antibody (Meridian Life Science W99008A) was diluted 1:8000 in blocking solution and added after four automated washing steps. After another wash, phosphatase substrate (Sigma S0942) was dissolved in substrate buffer per the instructions of the manufacturer and dispensed onto the plates. The plates were read at 405 nm on a PowerWave HT (BioTek).

VLP expression and HAI assays. Expression plasmids encoding the parenteral or mutated 1918 HA were co-expressed with N1 neuraminidase to produce VLPs in 293T cells (Yu et al., 2008a; Chen et al., 2007). Two days post-transfection, supernatants were collected. HAI assays were performed as described (World Health Organization Collaborating Centers for Reference and Research on Influenza) using VLPs (for 1918 H1N1 or H2) or live virus (all other influenza strains). Briefly, serially diluted antibodies were pre-incubated with eight hemagglutinating units of virus or VLP per well. Chicken red blood cells were added to a final concentration of 0.5%, and the plate was incubated on ice for 30 to 60 min.

In vivo antiviral effect of Ab 4K8. Female 8-week-old BALB/c mice were inoculated intranasally with 5×LD₅₀ in a 50 μL volume of the virulent reconstituted 1918 influenza virus. At 24 h after inoculation, mice were each administered 200, 20, or 2 μg (approximately 10, 1, or 0.1 mg/kg) of Ab 4K8 or an equal volume of polyclonal human IgG (Sigma) by the i.p. route in groups of 10 mice. Mice were observed for weight loss for 14 days. Subsets of four animals treated with Abs were euthanized on day 4 after inoculation, and whole lungs were homogenized in 1 mL of sterile PBS. Virus titers in lung tissue homogenates were determined by plaque titration in Madin-Darby Canine Kidney cell monolayer cultures and expressed as log₁₀ PFU/mL.

Isolation and characterization of antibody escape mutant viruses. The inventors selected new antibody escape mutant viruses by incubating virus with neutralizing antibodies followed by inoculation in 10-day-old embryonated chicken eggs, essentially as described (Caton et al., 1982; Yewdell et al., 1979). RNA was extracted from virus-infected allantoic fluid, then cDNA was generated by RT-PCR, cloned molecularly, and sequenced. The K166 escape mutations found in selected virus mutants were built back into 1918 VLPs for use in HAI assays to confirm that these alterations mediated escape from neutralization (Yu et al., 2008a).

Pyrosequencing. Total RNA was extracted from 30 million PBMCs (RNeasy kit, Qiagen). A first round PCR was performed for 35 cycles using BIOMED-2 V_{H}3 framework 1/J_{H} primers (van Dongen et al., 2003) and the OneStep RT-PCR kit (Qiagen). 454-specific adapters (Roche) were added by 10 cycles of a second round PCR. The PCR products were sequenced on a GS Junior instrument (Roche). The primers were cartridge-purified (Invitrogen); the sequences were CCATCAGCTGGGGGGTCCCTGAGACTCTCCTG (forward; SEQ ID NO:105) and CGCTCAGCTTACCTGAGGAGACGGTGACC (reverse; SEQ ID NO:106) for the first round and CGTATCGCCTCCCTCGCGCCATCAGCTGGGGGGTCCCTGAGACTCTCCTG (forward; SEQ ID NO:107) CTATGCGCCTTGCCAGCCCGCICAGCTTACCTGAGGAGACGGTGACC (reverse; SEQ ID NO:108) for the second round. The gene-specific elements are in bold; the key is underlined; Roche-specific primer A/B sequences are in italics. Sequence analysis was performed with IMGT/HighV-Quest (Brochet et al., 2008). IMGT output was analyzed in Access 2010 (Microsoft).

### B. Results

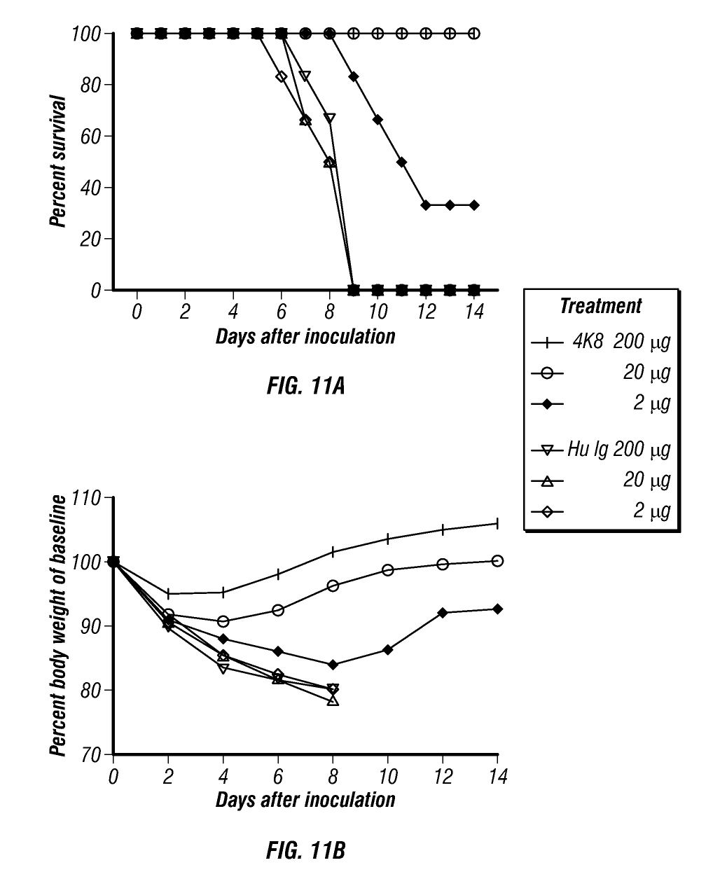

Hybridoma generation, antibody reactivity, animal studies. The inventors isolated a panel of five human monoclonal antibodies named 4A10, 2O10, 4K8, 6D9, and 2K11 from a single blood sample of a human subject. All antibodies showed HAI activity against both 1918 and 2009 H1N1 pandemic viruses, the related swine influenza viruses from 1930 and 1976, and the H1N1 virus from 1977, but not against strains from the 1940s or H1N1 strains after 1977 (Table 17). Ab 6D9 tested negative for functional activity against representative H2N2, H3N2, or H5N1 viruses by HAI (data not shown). Generally, Ab 4K8 was the most potent antibody, with an HAI activity<0.01 μg/mL against pandemic H1N1 viruses. The inventors selected this Ab for testing in a lethal mouse model of 1918 influenza virus infection (FIGS. 11A-B, Table 18). Ab 4K8 protected 6 out of 6 animals from death at both the highest and the intermediate dose levels and 2 out of 6 animals at the lowest dose (FIG. 11A). Ab 4K8 reduced lung virus titers as compared to the human IgG control by 3.1 log₁₀ PFU/mL at the 200 μg dose, 2.6 log₁₀ PFU/mL at the 20 μg dose, and still 1.3 log₁₀ PFU/mL at the 2 μg dose (Table PPPII).

TABLE 17
Specific HAI activity of human antibodies against
influenza viruses or VLPs

| | HAI activity of indicated Ab [μg/mL] | | | | |
|---|---|---|---|---|---|
| Influenza A strain | 4A10 | 2O10 | 4K8 | 6D9 | 2K11 |
| A/South Carolina/1/1918 wt (VLP) | 0.08 | 0.04 | <0.01 | 0.2 | 0.16 |
| A/South Carolina/1/1918 K166E | > | > | > | > | > |
| (VLP) | | | | | |
| A/South Carolina/1/1918 K166N | > | > | > | > | > |
| (VLP) | | | | | |
| A/South Carolina/1/1918 K166Q | > | > | > | > | > |
| (VLP) | | | | | |
| A/South Carolina/1/1918 K166R | 10 | > | > | 20 | > |
| (VLP) | | | | | |
| A/swine/Iowa/15/1930 | 0.63 | 0.08 | <0.01 | 0.4 | 0.16 |
| A/Weiss/1943 | > | > | > | > | > |
| A/L3/47 | > | > | > | > | > |
| A/New Jersey/11/1976 | 2.5 | 2.5 | 0.16 | 1.3 | 1.3 |
| A/USSR/92/1977 | 0.32 | 2.5 | 0.32 | 2.5 | 0.32 |
| A/New Caledonia/20/1999 | > | > | > | > | > |
| A/Brisbane/59/2007 | > | > | > | > | > |
| A/California/04/2009 | 0.16 | 0.32 | <0.01 | 0.7 | 0.63 |

The > symbol indicates that activity was not detected at the highest concentration tested, 20 μg/mL.

TABLE 18
Therapeutic efficacy of Ab 4K8 against virus replication in mice
inoculated with 1918 influenza A virus. Four mice were inoculated
intranasally with 5x LD₅₀ and administered 4K8 antibody or
human IgG i.p. 24 h later. Mice were euthanized on day 4 after
inoculation for the determination of lung titers

| | Dose | Mean lung virus titer* |
|---|---|---|
| Antibody | [μg/mouse] | [log₁₀ PFU/mL ± SD] |
| Ab 4K8 | 200 | 3.6 ± 0.2 |
| | 20 | 4.3 ± 0.3 |
| | 2 | 5.3 ± 0.2 |
| Human IgG control | 200 | 6.7 ± 0.1 |
| | 20 | 6.9 ± 0.0 |
| | 2 | 6.6 ± 0.4 |

*At the α = 0.025 level controlling the overall type I error at 7.5%, the lung homogenates of the 4K8 groups had lower virus titers than the human IgG control groups for all dose levels by the Wilcoxon signed-rank test (p = 0.01429 for the each level).

Generation and validation of escape mutations in epitopes recognized by these five antibodies. Ab 4K8 selected for a K166R mutation in the A/USSR/97/1977 context and a K166E mutation in the A/California/04/2009 H1N1 context. To confirm that these mutations alone were sufficient to confer escape, the inventors produced 1918 virus-like particles (VLPs) with mutant HA. The entire panel of antibodies was unable to inhibit hemagglutination of VLPs with K166E, K166N, or K166Q mutations (Table 17). Abs 4A10 and 6D9 showed modest HAI activity against VLPs with the K166R mutation, but not 2O10, 4K8, or 2K11 (Table 17). These data suggested that all five Abs recognized a common epitope, possibly with minor differences in the mode of binding between those five antibodies.

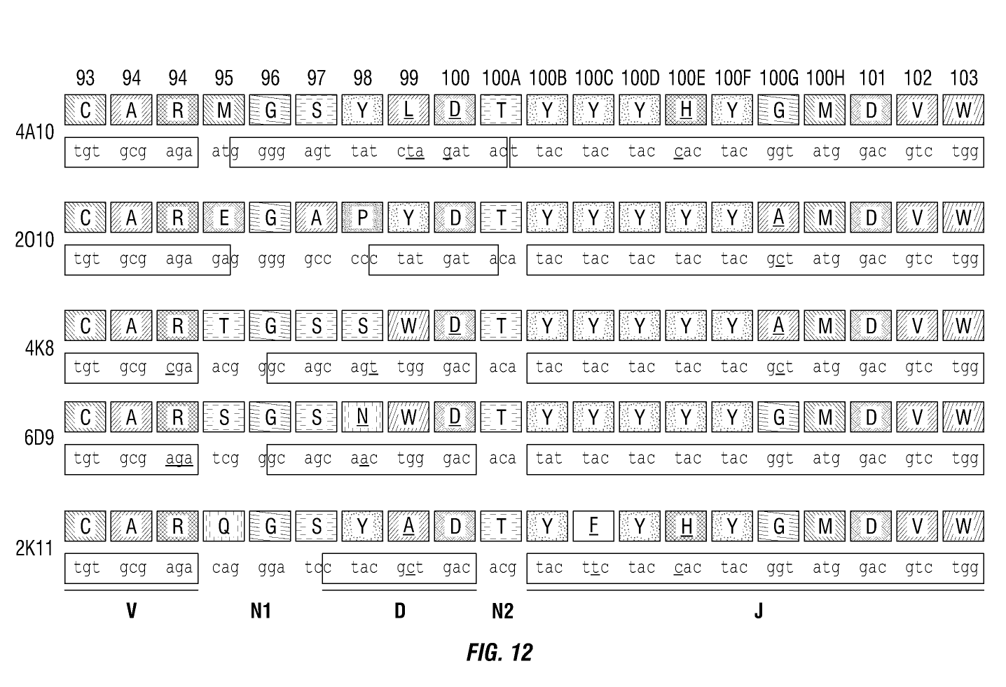

Sequence analysis of the antibody variable gene sequences in the hybridomas. Next, the inventors determined whether shared germline genes were the basis for the recognition of this epitope. Nucleotide sequence determination and sequence analysis of variable gene sequences (Table 19) using the international ImMunoGeneTics information system (IMGT) (Brochet et al., 2008) revealed that all of the five antibodies shared usage of the V_{H}3-7*01 gene and the J_{H}6*02 gene; furthermore, 4A10, 2O10, and 2K11 used the same V_{L}1-40*01 gene. Also, 4K8 and 6D9 used both V_{K}3-20*01 and J_{K}2. The D genes of these five antibodies were predicted to be of different origins except for 4K8 and 6D9, which shared the D6-13*01 gene (Table 19). These data suggested that 4K8 and 6D9 might be derived from the same B cell ancestor clone. Careful review of the junctional sequences showed that indeed 4K8 and 6D9 were clonally related, while 4A10, 2O10, and 2K11 were derived independently of each other and of the 4K8/6D9 clone (FIG. 12). Despite four different clonal origins, the CDR H3s of these antibodies were remarkably similar (FIG. 12); for instance the CDR H3 length of 18 amino acids was identical across this panel. The amino acids in positions 93-95, 96, 100, 100A, 100B, 100D, 100F, and 100H-103 were fully conserved. Interestingly, somatic mutations were shared between the antibodies, for example the tyrosine to histidine mutation in position 100E of 2K11 and 4A10 or the glycine to alanine mutation in position 100G of 2O10 and 4K8 (FIG. 12). Also, several common mutated amino acid residues were found despite differing sequences in the inferred germline origin sequence: the glycine in position 96 was encoded entirely by the N1 segment (2K11, 2O10), by the D segment (4A10), or by both (4K8/6D9). The S97 was encoded by the D segment alone (4A10, 4K8/6D9) or by both N1 and D1 (2K11). An aspartic acid was found in position 100 of 2K11 and 2O10 because of their germline; clones 4A10 and 4K8/6D9 acquired this aspartic acid through a somatic mutation, suggesting that this panel converged towards a consensus sequence (FIG. 12). Position 100A was predicted by IMGT to be encoded by the N2 segment alone (2K11, 4K8/6D9), by the D chain and the N2 segment (2O10), or by the D and the J chain (4A10; FIG. 12). No matter the origin, a threonine was found in all five CDR H3s in this position (FIG. 12). Residues from 100B onward were encoded by the J_{H}6 gene in all clones; four to six successive tyrosine residues are typically encoded by that germline gene segment (Zemlin et al., 2003), although somatic mutations were found in positions 100C (2K11) and 100E (4A10, 2K11).

TABLE 19
Genetic features of H1N1-specific human monoclonal antibodies

| | Heavy chain genes | | | Light chain genes | | |
|---|---|---|---|---|---|---|
| Ab | V_{H} | D | J_{H} | V_{K}/V_{L} | J_{K}/J_{L} | λ/κ |
| 4A10 | 3-7*01 | 3-16*02 | 6*02 | 1-40*01 | 2*01 or | λ |
| | | | | | 3*01 | |
| 2O10 | 3-7*01 | 3-22*01 | 6*02 | 1-40*01 | 1*01 | λ |
| 4K8 | 3-7*01 | 6-13*01 | 6*02 | 3-20*01 | 2 | κ |
| 6D9 | 3-7*01 | 6-13*01 | 6*02 | 3-20*01 | 2 | κ |
| 2K11 | 3-7*01 | 4-17*01 | 6*02 | 1-40*01 | 3*02 | λ |

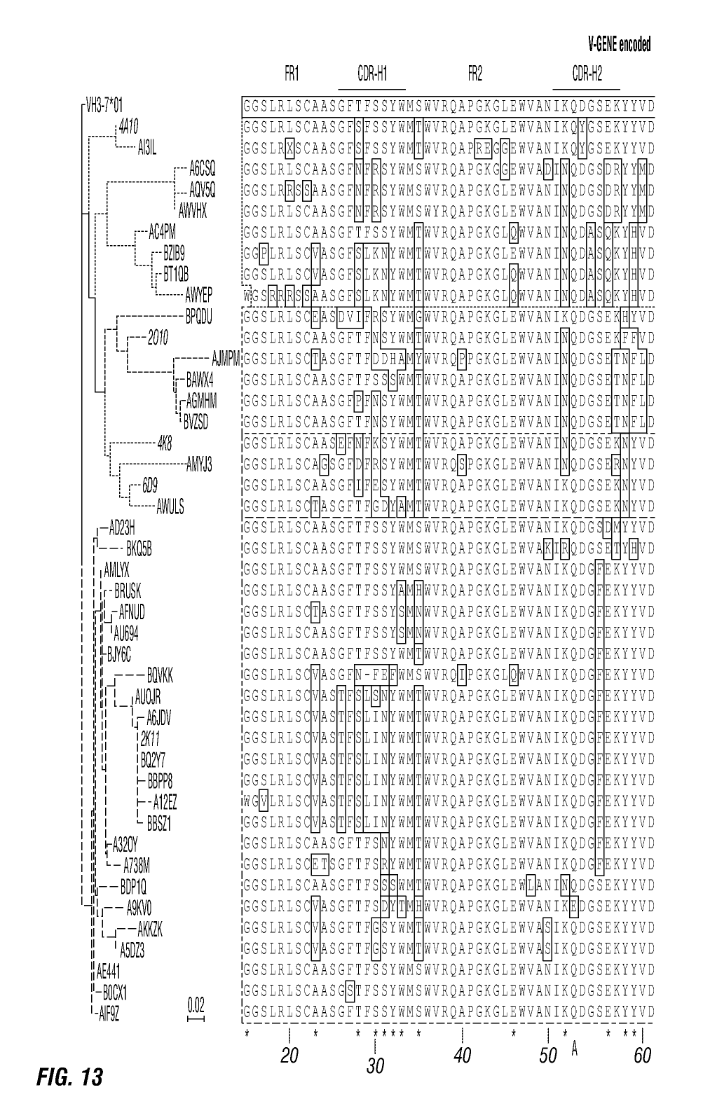

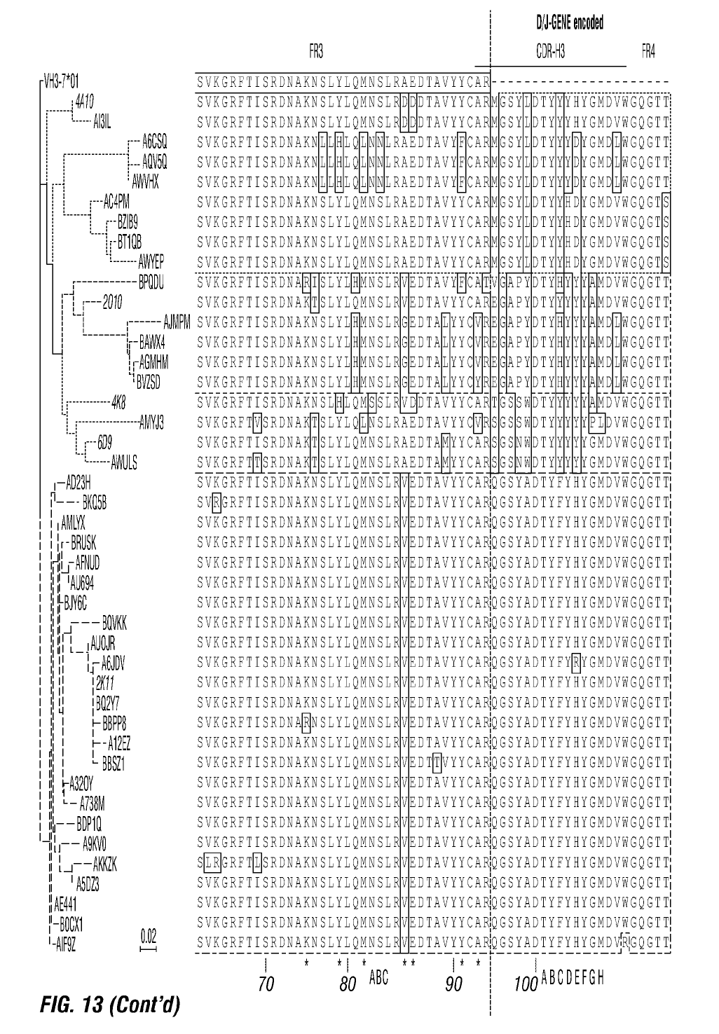

The inventors reviewed the variable gene encoded N-terminal part of heavy variable chains for common somatic mutations: 4A10 and 2K11 shared a threonine to serine mutation in position H28. All five antibodies shared a threonine instead of the germline serine in position H35 (FIG. 13). Abs 2O10 and 4K8 shared a lysine to asparagine mutation in position H52. Abs 2O10 and 6D9 mutated towards a threonine from an asparagine in position H76 (FIG. 13). Abs 2O10, 4K8, and 2K11 were found to have a valine instead of an alanine in position H84 (FIG. 13). An aspartic acid took the place of a glutamic acid in position H85 of Abs 4A10 and 4K8. Finally, both Abs 4A10 and 2K11 have a histidine to tyrosine mutation in position L34 of the λ chains in common.

Deep sequencing of the V_{H}3 gene encoded repertoire of this donor. The response of this donor towards 2009 H1N1 was dominated by antibodies encoded by the V_{H}3-7/J_{H}6 gene segments. The inventors hypothesized that this limited panel of five clones might represent only a small portion of the circulating antigenic site Sa-specific repertoire in this individual. To test this idea, the inventors extracted mRNA from total PBMCs from this donor six months after hybridoma generation and isolated antibody genes using an RT-PCR amplification specific for V_{H}3. The inventors used pyrosequencing to delineate the entire V_{H}3 repertoire. The inventors identified sister clones of the above antibodies based on shared V/J usage and identical VDJ junctions to more fully define the genetic diversity within these clones in circulating cells. The inventors obtained 26.2 megabases of data including 80,687 sequences that passed filter reads. The sequences had an average length of 325 base pairs after removal of primer sequences. Analysis with IMGT identified 60,484 of those sequences as productive; 60,447 belonged to the V_{H}3 germline. V_{H}3-23 and V_{H}3-11 each accounted for 17% of productive V_{H}3 sequences (the first IMGT assignment was used in case of ambiguities), V_{H}3-30 for 13%, and V_{H}3-7 for 9%. A total of 1,917 V_{H}3-7/J_{H}6 IgH were identified, of which 203 shared a CDR H3 length of 18 amino acids with the five neutralizing influenza antibodies the inventors had isolated. Of these 203 sequences, 138 were non-redundant on the protein level belonging to a total of 69 clones based on review of the VDJ junctions. Eight of those sequences belonged to the 4A10 lineage (five to clone 2O10, two to clone 4K8/6D9, and 23 to clone 2K11) meaning that every single clone was still found in the peripheral blood of the individual six months after the initial blood draw for hybridoma generation. Since residues DTY at positions 100-100B were completely conserved across all antibodies, the inventors screened the remaining clones for the presence of this motif, but none of them shared this DTY motif in CDR H3, suggesting that the inventors likely had identified most of the clones of the V_{H}3-7/J_{H}6 gene segment-encoded Abs that recognized the influenza HA Sa site.

Intraclonal sequence divergence of the 2K11 clone. While the inventors identified sequences highly related to those of Abs 4A10, 2O10, or 4K8/6D9, none of the sequences from the high throughput analysis was completely identical to those in the original clones. Remarkably, the inventors were able to recover a sequence that was virtually identical to the 2K11 hybridoma cell IgH sequence in the high throughput sequence BQ2Y7. This occurrence may have been linked to the fact that sequences recovered that were related to Ab 2K11 represented by far the biggest clonal family identified and thus provided a more comprehensive snapshot of the evolution of this antibody. Within the panel related to Ab 2K11, the AE441 and AIF9Z sequences embodied essentially a germline state with just a single non-silent somatic mutation in the variable gene encoded amino acid sequence, the valine in position 84. On the other hand, the Ab 2K11 sequence itself and related sequences were highly mutated, particularly in the CDR H1, with up to five changes in amino acid sequence. While both germline states and highly mutated states were present simultaneously in the peripheral blood, sequences representing many of the intermediate predicted steps were not detected. For example, it was not clear in what order the mutations within CDR H1 of the hybridoma 2K11 occurred except that S31N probably occurred first because it was present in A32OY by itself. Also the S30I mutation probably occurred last within CDR H1 because AUOJR contained all of the other mutations except the aforementioned one. Since the other three clones had fewer representatives than the 2K11 clone, the order of mutations would be more difficult to establish for those clones. Nevertheless, sequence divergence from the germline sequence was readily apparent. Notably, insertion/deletion events seemed to play a minor role in this antibody panel with only sequence BQVKK within the 2K11 clone bearing evidence of a three base pair deletion leading to the loss of a single amino acid residue within CDR H1.

Interclonal sequence convergence. The inventors defined convergence as the same altered amino acid introduced by somatic mutation present in two or more independent clones. Despite sequence divergence within the individual clones, sequence comparison revealed many further examples of interclonal sequence convergence not evident in the hybridoma sequences such as valine in H23 (members of clones 4A10/2K11) or threonine in the same position (2O10, 4K8/6D9, 2K11), leucine in H29 (4A10/2K11), asparagine in H31 (4A10/2K11), glutamine in H46 (4A10/2K11), asparagine in H58 (2O10/4K8/6D9), histidine in H59 (4A10, 2K11), and several others (FIG. 13). Overall, there were 20 positions within the V_{H} protein sequence with evidence of convergence. Only eight of those 20 positions were found within CDR H1, H2, or the V-GENE encoded portion of H3, so 12 convergence positions were located in the framework regions. Remarkably, a subclone each within two different clones converged towards a set of similar somatic mutations within the CDR H1 (amino acids SUN in positions H28-31 in the 2K11 clones and amino acids SLKN in the same positions of the 4A10 lineage).

### C. Discussion

Glycosylation within site Sa does not always confer escape from neutralization. The inventors describe a panel of four independent clones of human antibodies. Like Ab 2D1, they were Sa site antibodies based on the selection of escape mutations in position K166 and that showed potent HAI activity against pandemic H1N1 influenza (Krause et al., 2010; Xu et al., 2010b; Yu et al., 2008a). Unlike Ab 2D1 however, this panel had HAI activity against USSR/77 H1N1 as well. This finding is striking because the USSR virus possesses three predicted N-linked glycosylation sites within its Sa antigenic site (Xu et al., 2010b). Glycosylation at these sites is thought to shield HA from neutralization (Wei et al., 2010; Xu et al., 2010b). In support of this model, experimental introduction of glycosylation sites into the 1918 or 2009 HAs conferred resistance to neutralization by antiserum elicited by the unglycosylated HAs (Wei et al., 2010). The fact that neither the 1918 virus, nor the 2009 pandemic virus possess glycosylation sites within the Sa antigenic site, while human H1N1 viruses acquired N-linked glycosylation over the course of the 20^{th} century, likely explains, at least in part, why elderly people had preexisting cross-reactive humoral immunity against the 2009 pandemic virus (Wei et al., 2010; Xu et al., 2010b). The fact that V_{H}3-7/J_{H}6 Abs inhibited USSR virus, despite the presence of the glycosylation sites, demonstrated that the presence of glycosylation sites within site Sa did not always confer escape from Sa-specific antibodies.

Redundancy of the immune response. Interestingly, this V_{H}3-7/J_{H}6 panel of four independent clones was derived from a single donor; her response to pandemic 2009 H1N1 may have been V_{H}3-7/J_{H}6 dominant and oligoclonal since the inventors were only able to generate another H1N1 globular head antibody, Ab 5J8, from this donor that selected for escape mutations along the receptor-binding pocket (unpublished data). The redundancy of this response may ensure adequate neutralization of a given pathogen such as influenza A virus. This redundancy may be quite common, but may only be detected more frequently through advances in antibody engineering and sequencing technology.

Same genes suggest a common epitope. Wrammert et al. described cross-reactive human Abs in subjects convalescing from infection with 2009 H1N1 pandemic virus (Wrammert et al., 2011); this work did not feature neutralizing V_{H}3-7/J_{H}6 antibodies—potentially due to a limited number of antibodies. While a similar genetic makeup (like V_{H}3-7/J_{H}6) suggests a common epitope, the Sa antigenic site can likely be reached from a variety of different germline configurations such as V_{H}3-7/J_{H}6 or the V_{H}2-70 of the 2D1 antibody (Krause et al., 2010; Krause et al., 2011; Yu et al., 2008a).

The V_{H}3-7/J_{H}6 antibodies described here showed strongest HAI activity against 1918 VLPs of a virus that circulated well before the birth of this donor and thus could not have served as the inciting agent. In fact, given that this donor was born between 1957 and 1977 when H1N1 did not circulate in humans, subtype H1N1 infection was unlikely to have been the cause of her first exposure to influenza or original antigenic sin (Davenport et al., 1953). These antibodies could have been created in response to vaccination with the 1976 swine influenza virus (Krause, 2006) or vaccination or infection with a USSR/77-like virus or the 2009 H1N1 virus. Since these antibodies in general seemed to neutralize 2009 H1N1 best among those three candidates, the inventors hypothesized that these antibodies were created in response to the 2009 virus and that the process of several antibodies independently hitting the same epitope with a similar genetic makeup was entirely stochastic.

Intraclonal sequence divergence and interclonal sequence convergence. The ultra deep sequencing in this study revealed that the V_{H}3-7/J_{H}6 clones represented large circulating phylogenies. Since pyrosequencing was performed on PBMCs isolated six months after the initial hybridoma generation, the phylogenies seemed to persist for at least several months in the peripheral blood. B-cells that are not stimulated because they do not express high-affinity antibody are supposed not to undergo further proliferation (French et al., 1989). Still, wide divergence of sequences from essentially unmutated germline states to extensively mutated sequences was found within clones in the peripheral blood of this donor. Persistent low-affinity memory populations may aid in the immunologic response towards a related HA antigen that the individual subsequently encounters (Herzenberg et al., 1980; Fish et al., 1989).

B cell development has been traced by molecular analysis of single cells picked from histological sections in human germinal centers (Kuppers et al., 1993; Tierens et al., 1999) and spleen (Tierens et al., 1999; Dunn-Walters et al., 1995) showing the presence of both naïve and memory B cells. An alternative method to track the development of single memory-lineage B cells has been the use of a specific anti-idiotypic antibody E4, which recognizes a canonical V region (Liu et al., 1996). In the mouse study by Liu et al, this E4⁺ pool is derived from fewer than five canonical precursors. A lack of shared somatic mutations across clonally-related cells by day 13 indicates that the selective expansion of mutant subclones typical of memory responses did not yet occur as it did in the inventors' panel of antibodies (Liu et al., 1996). The strength of the inventors' study was the combination of functional data from human hybridoma technology with ultra deep sequencing. A limitation of the deep sequencing was that the inventors were not able to document the concurrent evolution of the corresponding light chains, although the hybridoma technology allowed us to find at least one hybridoma per clone with a matching heavy and light chain, adding further evidence that these hybridomas represented four distinct clones.

Despite different clonal origins within the VDJ junction, these four independent clones converged towards common amino acid residues within this junction and throughout the shared V_{H}3-7 gene. This suggested a strong selection for optimal binding sites across clones. Since three clones also share the V_{L}1-40 gene, but different J genes, CDRs H1, H2, H3, L1, and L2 would be expected to make contacts with the HA. Given the frequency of mutations within the CDR H1 segment and the conservation of the typically very variable CDR H3, the inventors hypothesize that those two loops would be especially critical in the antibody-antigen interaction. The majority of the convergence positions are within framework regions; these residues can be at the surface, the core, or the heavy chain-light chain interface of the antibody. Core mutations partially determine stability (Clark et al., 2006) and may change the conformation of adjacent and distant CDR loops (Krause et al., 2011).

Sequence convergence has been found in a variety of proteins across individuals and species. Antibodies of the human V_{H}1-69 germline from diverse phage display libraries, plasmablasts, and EBV-transformed B cells neutralize the stem of H1N1 and H5N1 influenza viruses (Wrammert et al., 2011; Throsby et al., 2008; Ekiert et al., 2009; Sui et al., 2009; Corti et al., 2010). Likewise, the phage-derived HIV antibodies Fab 8066 and D5 from entirely different antibody libraries and panning procedures show convergence in their sequences and also in the conformation of the CDR H2 loop (Gustchina et al., 2010). Indeed, pyrosequencing of zebrafish antibodies showed evidence of convergence, in which different individuals made the same antibody (Weinstein et al., 2009), but this was not correlated with functional data. Sequence convergence is not only seen in antibodies across individuals of the same species (or—as demonstrated here—within a single individual), but even in proteins that serve a similar purpose across distinct mammalian species (Liu et al., 2010). With continued progress in B cell technology, there will likely be further discoveries of converging sequence-related antibodies from one or multiple individuals that share common epitopes. Once common germline gene responses to defined epitopes of pathogens are better understood, it might be possible to diagnose an infection of an individual based on the immune repertoire documented by ultra deep sequencing.

All of the compositions and methods disclosed and claimed herein can be made and executed without undue experimentation in light of the present disclosure. While the compositions and methods of this invention have been described in terms of preferred embodiments, it will be apparent to those of skill in the art that variations may be applied to the compositions and methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### VIII. REFERENCES

The following references, to the extent that they provide exemplary procedural or other details supplementary to those set forth herein, are specifically incorporated herein by reference.
U.S. Pat. No. 3,817,837
U.S. Pat. No. 3,850,752
U.S. Pat. No. 3,939,350
U.S. Pat. No. 3,996,345
U.S. Pat. No. 4,196,265
U.S. Pat. No. 4,275,149
U.S. Pat. No. 4,277,437
U.S. Pat. No. 4,366,241
U.S. Pat. No. 4,472,509
U.S. Pat. No. 4,554,101
U.S. Pat. No. 4,680,338
U.S. Pat. No. 4,816,567
U.S. Pat. No. 4,867,973
U.S. Pat. No. 4,938,948
U.S. Pat. No. 5,021,236
U.S. Pat. No. 5,141,648
U.S. Pat. No. 5,196,066
U.S. Pat. No. 5,563,250
U.S. Pat. No. 5,565,332
U.S. Pat. No. 5,856,456
U.S. Pat. No. 5,880,270
“Antibodies: A Laboratory Manual,” Cold Spring Harbor Press, Cold Spring Harbor, N.Y., 1988.
Abbondanzo et al., Am. J. Pediatr. Hematol. Oncol., 12(4), 480-489, 1990.
Abhinandan and Martin, Mol. Immunol., 45:3832-3839, 2008.
Allred et al., Arch. Surg., 125(1), 107-113, 1990.
Atherton et al., Biol. of Reproduction, 32, 155-171, 1985.
Beidler et al., J. Immunol., 141(11):4053-4060, 1988.
Bernstein et al., J. Infect. Dis., 197:667-675, 2008.
Brochet et al., Nucleic Acids Res., 36:W503-508, 2008.
Brown et al., J. Immunol. Meth., 12;130(1), :111-121, 1990.
Brownlee and Fodor, Philos. Trans. R Soc. Lond. B Biol. Sci., 356:1871-1876, 2011.
Campbell, In: Monoclonal Antibody Technology, Laboratory Techniques in Biochemistry and Molecular Biology, Vol. 13, Burden and Von Knippenberg, Eds. pp. 75-83, Amsterdam, Elsevier, 1984.
Capaldi et al., Biochem. Biophys. Res. Comm., 74(2):425-433, 1977.
Caton et al., Cell, 31:417-427, 1982.
Chen et al., J. Virol., 81:7111-7123, 2007.
Clark et al., J. Immunol., 177:333-340, 2006.
Connor et al., Virology, 205:17-23, 1994.
Corti et al., J. Clin. Invest., 120:1663-1673, 2010.
Corti et al., Science, 333(6044):850-856, 2011.
Davenport et al., J. Exp. Med., 98:641-656, 1953.
De Jager et al., Semin. Nucl. Med. 23(2), 165-179, 1993.
Dholakia et al., J. Biol. Chem., 264, 20638-20642, 1989.
Doolittle and Ben-Zeev, Methods Mol. Biol., 109, :215-237, 1999.
Dunn-Walters et al., J. Exp. Med., 182:559-566, 1995.
Ekiert et al., Science, 324:246-251, 2009.
EP Application 125,023
EP Application 171,496
EP Application 173,494
EP Application 184,187
Epstein, J. Infect. Dis., 193:49-53, 2006.
Fish et al., J. Exp. Med., 170:1191-1209, 1989.
French et al., Science, 244:1152-1157, 1989.
Gefter et al., Somatic Cell Genet., 3:231-236, 1977.
Goding, In: Monoclonal Antibodies: Principles and Practice, 2d ed., Orlando, Fla., Academic Press, 60-61, 65-66, 71-74, 1986.
Gulbis and Galand, Hum. Pathol. 24(12), 1271-1285, 1993.
Gustchina et al., PLoS Pathog, 6:e1001182, 2010.
Hehme et al., Med. Microbiol. Immunol., 191:203-208, 2002.
Herzenberg et al., J. Exp. Med., 151:1071-1087, 1980.
Jones et al., Nature, 321:522-525, 1986.
Khatoon et al., Ann. of Neurology, 26, 210-219, 1989.
King et al., J. Biol. Chem., 269, 10210-10218, 1989.
Knossow and Skehel, Immunology, 119:1-7, 2006.
Kobasa et al., Nature 445:319-323, 2007.
Kohler and Milstein, Eur. J. Immunol., 6, 511-519, 1976.
Kohler and Milstein, Nature, 256, 495-497, 1975.
Krause et al., J. Virol., 84:3127-3130, 2010.
Krause et al., MBio., 2(1):e00345-10, 2011.
Krause, Emerg. Infect. Dis., 12:40-43, 2006.
Kuppers et al., EMBO J., 12:4955-4967, 1993.
Kyte and Doolittle, J. Mol. Biol., 157(1):105-132, 1982.
Lagerkvist et al., Biotechniques, 18:862-869, 1995.
Larkin et al., Bioinformatics, 23:2947-2948, 2007.
Lefranc, Nucleic Acids Res., 31:307-310, 2003.
Liu et al., Curr. Biol., 20:R53-54, 2010.
Liu et al., J. Exp. Med., 183:2053-2063, 1996.
Manicassamy et al., PLoS Pathog., 6:e1000745, 2010.
Matrosovich et al., J. Virol., 74:8502-8512, 2000.
Morrison, Science, 229(4719):1202-1207, 1985.
Nakamura et al., In: Enzyme Immunoassays: Heterogeneous and Homogeneous Systems, Chapter 27, 1987.
Okuno et al., J. Virol., 67:2552-2558, 1993.
O'Shannessy et al., J. Immun. Meth., 99, 153-161, 1987.
Owens and Haley, J. Biol. Chem., 259, 14843-14848, 1987.
Pappas et al., PLoS One, 5:e11158, 2010.
PCT Application PCT/US86/02269
PCT Application WO 86/01533
Pommie et al., J. Mol. Recognit., 17:17-32, 2004.
Posner et al., Hybridoma 6, 611-625, 1987.
Potter and Haley, Meth. Enzymol., 91, 613-633, 1983.
Sblattero and Bradbury, Immunotechnology, 3:271-278, 1998.
Shaw et al., J. Natl. Cancer Inst., 80(19):1553-1559, 1988.
Smith et al., Nat. Protoc., 4:372-384, 2009.
Stevens et al., Science, 303:1866-1870, 2004.
Sui et al., Nat. Struct. Mol. Biol., 16:265-273, 2009.
Sun et al., J. Steroid Biochem., 26(1):83-92, 1987.
Tang et al., J. Biol. Chem., 271:28324-28330, 1996.
Throsby et al., PLoS One, 3:e3942, 2008.
Tierens et al., Blood, 93:226-234, 1999.
van Dongen et al., Leukemia, 17:2257-2317, 2003.
Verhoeyen et al., Science, 239(4847):1534-1536, 1988.
Viswanathan et al., PLoS One, 5:e13768, 2010.
Wawrzynczak & Thorpe, In: Immunoconjugates, Antibody Conjugates In Radioimaging And Therapy Of Cancer, Vogel (Ed.), NY, Oxford University Press, 28, 1987.
Wei et al., Sci. Transl. Med., 2:24ra21, 2010.
Weinstein et al., Science, 324:807-810, 2009.
Whittle et al., Proc. Natl. Acad. Sci., USA, 108(34):14216-14221, 2011.
Wood et al., J. Clin. Lab. Immunol., 17(4):167-171, 1985.
World Health Organization Collaborating Centers for Reference and Research on Influenza. Concepts and Procedures for Laboratory-Based Influenza Surveillance. Centers for Disease Control and Prevention, Atlanta, Ga.
Wrammert et al., J. Exp. Med., 208:181-193, 2011.
Wrammert et al., Nature, 453:667-671, 2008.
Wright et al., In: Fields virology, Knipe and Howley (Eds.), Wolters Kluwer Health/Lippincott Williams & Wilkins, Philadelphia, 1699, 2007.
Wu et al., Proteins 16:1-7, 1993.
Xu et al., J. Virol., 84:1715-1721, 2010a.
Xu et al., Science, 328:357-360, 2010b.
Yamada et al., Virology, 138:276-286, 1984.
Yewdell et al., Nature, 279:246-248, 1979.
Yoshida, et al., PLoS Pathog., 5:e1000350, 2009.
Yu et al., Nature, 455:532-536, 2008a.
Yu et al., J. Immunol. Methods, 336:142-151, 2008b.
Zemlin et al., J. Mol. Biol., 334:733-749, 2003.

1. A human monoclonal antibody that:
(a) binds to globular head region of influenza virus hemagglutinin;
(b) cross-reacts between Group 1 and Group 2 influenza viruses; and
(c) neutralizes virus and/or inhibits influenza hemagglutination.

2. The antibody of claim 1, further cross-reacting with multiple Group 1 influenza viruses.

3. The antibody of claim 2, wherein the multiple Group 1 influenza viruses comprise H1, H2, H5 and H9.

4. The antibody of claim 1, further cross-reacting with multiple Group 2 influenza viruses.

5. The antibody of claim 4, wherein the multiple Group 2 influenza viruses are H3 and H7.

6. The antibody of claim 1, wherein the antibody neutralizes influenza virus.

7. The antibody of claim 1, wherein the antibody inhibits influenza hemagglutination.

8. The antibody of claim 1, wherein the antibody neutralizes virus and inhibits influenza hemagglutination.

9. The antibody of claim 1, wherein said antibody is a recombinant antibody.

10. The antibody of claim 1, wherein the antibody binds the same epitope as 8F24.

11. The antibody of claim 1, wherein the antibody binds the same epitope as 3E22 or 5I17.

12. The antibody of claim 1, wherein the heavy/light chain variable region sequences are selected from the group consisting of SEQ ID NO:1/3, SEQ ID NO:5/7 and SEQ ID NO:9/11.

13. A human monoclonal antibody that binds to a long alpha helix region of influenza virus hemagglutinin and cross-reacts between multiple H1 influenza viruses.

14. The antibody of claim 13, further cross-reacting with multiple Group 1 influenza viruses subtypes and is Group 1-specific.

15. The antibody of claim 14, wherein the multiple Group 1 influenza viruses comprise H1, H2 and H5.

16. The antibody of claim 13, wherein the antibody exhibits virus neutralization activity.

17. The antibody of claim 13, wherein said antibody is a recombinant antibody.

18. The antibody of claim 14, further cross-reacting with multiple Group 1 influenza viruses and are Group 2-cross-reactive.

19. The antibody of claim 13, wherein the heavy/light chain variable region sequences are selected from the group consisting of SEQ ID NO: 13/15, SEQ ID NO:29/31 and SEQ ID NO:33/35.

20. A human monoclonal antibody that binds to a long alpha helix region of influenza virus hemagglutinin and cross-reacts between multiple H3 influenza viruses.

21. The antibody of claim 20, further cross-reacting with multiple Group 2 influenza viruses and is Group 2-specific.

22. The antibody of claim 21, wherein the multiple Group 2 influenza viruses comprise H3 and H7, H3 and H9, or H3, H7 and H9.

23. The antibody of claim 20, wherein the antibody exhibits virus neutralization activity.

24. The antibody of claim 20, wherein said antibody is a recombinant antibody.

25. The antibody of claim 20, wherein the antibody is designated 5J4 or 1C23.

26. A human monoclonal antibody that binds to the stalk domain of the influenza virus hemagglutinin and cross-reacts between multiple H1 and multiple Group 1 influenza viruses, wherein the heavy/light chain variable region sequences are selected from the group consisting of SEQ ID NO: 17/19, SEQ ID NO: 21/23 and SEQ ID NO: 25/27.

27. The antibody of claim 26, wherein the multiple Group 1 influenza viruses comprise H1 and H2, H1 and H5, or H1, H2 and H5.

28. The antibody of claim 26, wherein the antibody is Group 1-specific.

29. The antibody of claim 26, wherein the antibody is Group 2-cross-reactive.

30. An immunogen consisting essentially of:
(a) a long alpha helix (LAH) region of influenza virus hemagglutinin; and
(b) a trimerization domain.

31-87. (canceled)
